SEQUENCE LISTING (1 sequence)

SEQ ID NO: 1 - protein, 627 aa, Homo sapiens
MEGLAGYVYKAASEGKVLTLAALLLNRSESDIRYLLGYVSQQGGQRSTPLIIAARNGHAKVVRLLLEHYRVQTQQTGTVRFDGYVIDGATALWCAAGAGHFEVVKLLVSHGANVNHTTVTNSTPLRAACFDGRLDIVKYLVENNANISIANKYDNTCLMIAAYKGHTDVVRYLLEQRADPNAKAHCGATALHFAAEAGHIDIVKELIKWRAAIVVNGHGMTPLKVAAESCKADVVELLLSHADCDRRSRIEALELLGASFANDRENYDIIKTYHYLYLAMLERFQDGDNILEKEVLPPIHAYGNRTECRNPQELESIRQDRDALHMEGLIVRERILGADNIDVSHPIIYRGAVYADNMEFEQCIKLWLHALHLRQKGNRNTHKDLLRFAQVFSQMIHLNETVKAPDIECVLRCSVLEIEQSMNRVKNISDADVHNAMDNYECNLYTFLYLVCISTKTQCSEEDQCKINKQIYNLIHLDPRTREGFTLLHLAVNSNTPVDDFHTNDVCSFPNALVTKLLLDCGAEVNAVDNEGNSALHIIVQYNRPISDFLTLHSIIISLVEAGAHTDMTNKQNKTPLDKSTTGVSEILLKTQMKMSLKCLAARAVRANDINYQDQIPRTLEEFVGFH

(19) United States
(12) Patent Application Publication
(10) Pub. No.: US 2023/0148299 A1
(43) Pub. Date: May 11, 2023

(54) FEM1B PROTEIN BINDING AGENTS AND USES THEREOF

(71) Applicant: The Regents Of The University Of California, Oakland, CA (US)

(72) Inventors: Michael Rape, El Cerrito, CA (US); Daniel K. Nomura, Walnut Creek, CA (US); Nathaniel Henning, Berkeley, CA (US); Andrew Manford, Berkeley, CA (US)

(21) Appl. No.: 17/802,476

(22) PCT Filed: Mar. 8, 2021
(86) PCT No.: PCT/US2021/021347

§ 371 Date: Aug. 25, 2022

Related U.S. Application Data
(60) Provisional application No. 62/987,304, filed on Mar. 9, 2020.

(51) Int. Cl. C07D 495/14

(52) U.S. Cl. CPC C07D 495/14

(57) ABSTRACT

Disclosed herein, inter alia, are compounds for binding FEMIB protein and uses thereof. In an aspect, provided input pulldown herein is a pharmaceutical composition including a compound as described herein and a pharmaceutically acceptable excipient.

### CROSS-REFERENCES TO RELATED APPLICATIONS

This application claims the benefit of U.S. Provisional Application No. 62/987,304, filed Mar. 9, 2020, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 052103-522001WO_Sequence_Listing_ST25, created Mar. 1, 2021, 5,777 bytes, machine format IBM-PC, MS Windows operating system, is hereby incorporated by reference.

### BACKGROUND

Metazoan development relies on carefully balanced transcriptional networks to generate the more than 200 cell types of an adult organism. Stem cells, which can either self-renew to generate more progenitors or differentiate into specialized cell types, are at the apex of this intricate program, and their defective homeostasis gives rise to many pediatric diseases (Avior et al., 2016; Nusse and Clevers, 2017). As stem cells support tissue regeneration and repair throughout the lifetime of an organism, aberrant stem cell maintenance has also been linked to tumorigenesis and tissue degeneration (Almada and Wagers, 2016). Disclosed herein, inter alia, are solutions to these and other problems in the art.

### BRIEF SUMMARY

In an aspect, provided herein is a compound having the formula:

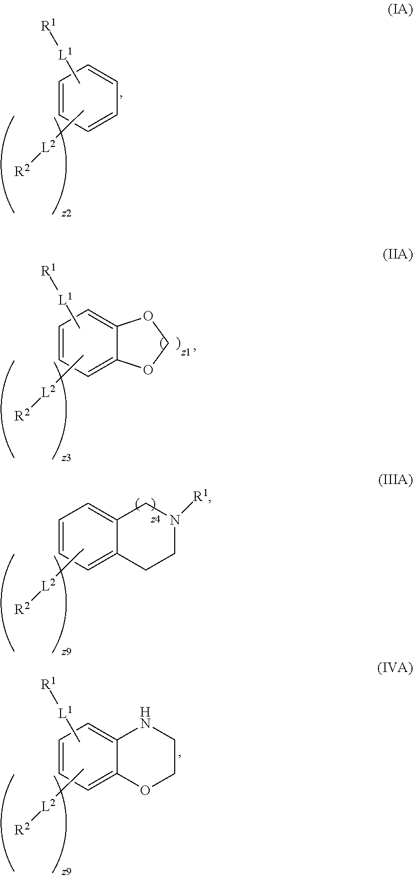

or a pharmaceutically acceptable salt thereof.

R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is an electrophilic moiety.

The symbol z1 is 1 or 2. The symbol z2 is 0 to 5. The symbol z3 is 0 to 3. The symbol z4 is 0 or 1. The symbols z5 and z9 are each independently an integer from 0 to 4.

In an aspect, provided herein is a compound having the formula:

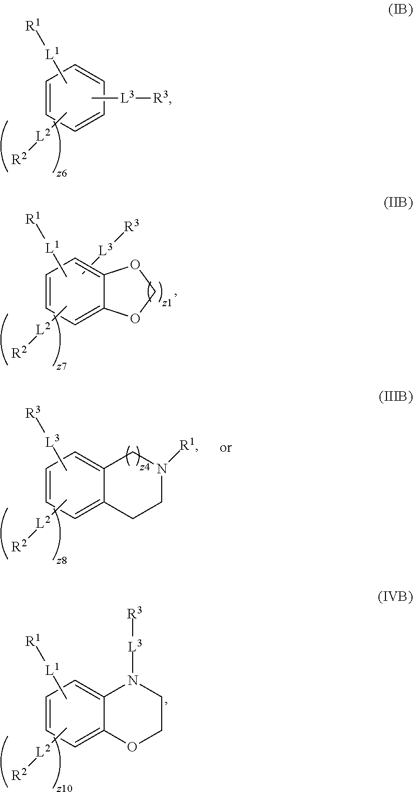

or a pharmaceutically acceptable salt thereof.

R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is independently a bond, —S(O)₂—, —N(R¹⁰²)—, —O—, —S—, —C(O)—, —C(O)N(R¹⁰²)—, —N(R¹⁰²)C(O), —N(R¹⁰²)C(O)NH—, —NHC(O)N(R¹⁰²)—, —C(O)O—, —OC(O)}, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is an electrophilic moiety.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is a target protein binding moiety.

The symbol z1 is 1 or 2. The symbol z4 is 0 or 1. The symbol z6 is 0 to 4. The symbol z7 is 0 to 2. The symbols z8 and z10 are each independently an integer from 0 to 3.

In an aspect, provided herein is a compound selected from a group consisting of:

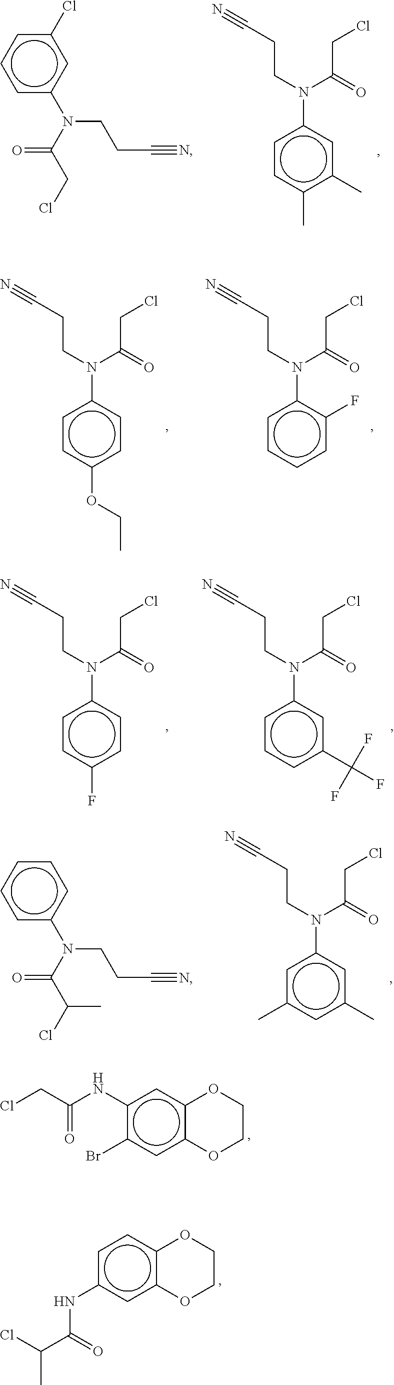

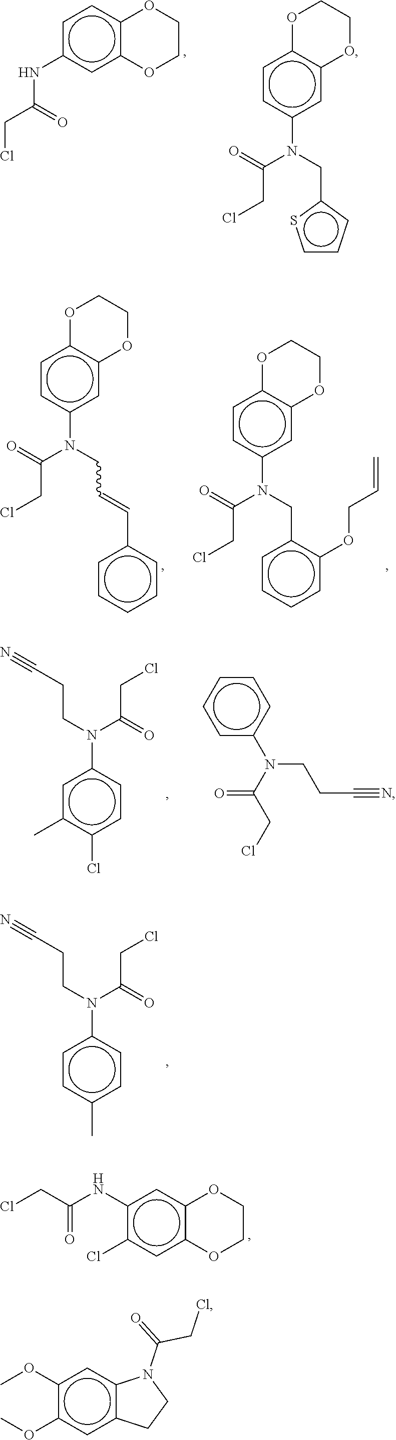

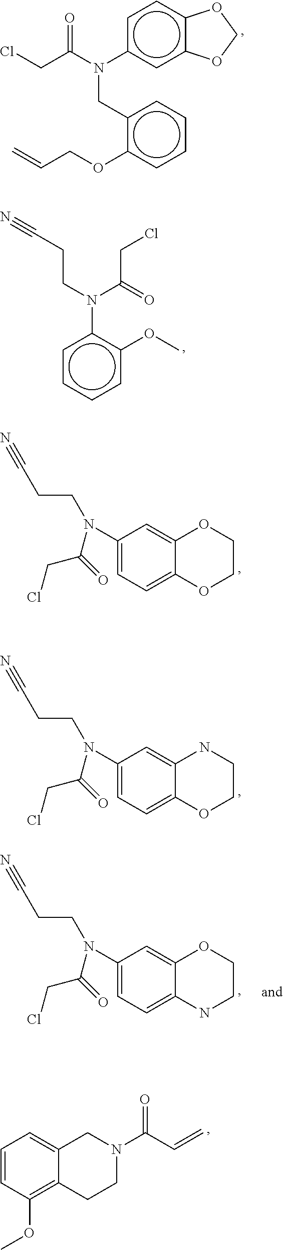

or a pharmaceutically acceptable salt thereof.

In an aspect, provided herein is a pharmaceutical composition including a compound as described herein and a pharmaceutically acceptable excipient.

In an aspect, provided herein is a method of treating a disease in a subject in need thereof, the method including administering a therapeutically effective amount of FEM1B Cys 186 covalent inhibitor.

In an aspect, provided herein is a method of treating a disease in a subject in need thereof, the method including administering a therapeutically effective amount of a compound having the structure: FCIM-L³-R³.

FCIM is a FEM1B Cys 186 covalent inhibitor moiety.

R³ is a target protein binding moiety.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect, provided herein is a FEM1B protein including an amino acid corresponding to Cys 186. Amino acid corresponding to Cys 186 is covalently bound to a (i) FEM1B Cys 186 covalent inhibitor, or (ii) a compound having the structure: FCIM-L³-R³.

FCIM is a FEM1B Cys 186 covalent inhibitor moiety.

R³ is a target protein binding moiety.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCH₁, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

FCIM is covalently bound to the Cys 186.

### BRIEF DESCRIPTION OF THE DRAWINGS

FIGS. 1A-1G. FEM1B and FNIP1 are central regulators of metabolism. FIG. 1A: FEM1B does not strongly affect mTORC1 signaling in myoblasts. C2C12 myoblasts were depleted of FEM1B, FNIP1, or combinations thereof. Cells were starved, before amino acids were added to rapidly turn on mTORC1. mTORC1 activity was monitored by measuring levels of phosphorylated S6 kinase by gel electrophoresis and Western blotting. FIG. 1B: FNIP1 binds AMPK in myoblasts. ^{FLAG}FNIP1 was expressed in C2C12 myoblasts, and binding partners were determined by affinity-purification and mass spectrometry. FIG. 1C: FEM1B does not strongly affect AMPK signaling. C2C12 myoblasts were depleted of FEM1B, FNIP1, or combinations thereof, and AMPK signaling was monitored by measuring phosphorylated ACC and phosphorylated AMPK by gel electrophoresis and Western blotting. FIG. 1D: Depletion of FEM1B reduces the extracellular acidification rate, which is partially rescued by co-depletion of FNIP1. Depletion of FEM1B or FNIP1 do not affect the activity of the electron transport chain, if pyrvate is added to cells. The oxygen consumption rate (OCR) was measured for C2C12 cells transfected with the indicated siRNAs using a Seahorse Analyzer. FIG. 1E: FEM1B depletion does not inhibit the mitochondrial electron transfer chain per se. FIG. 1F: FEM1B depletion inhibits glucose uptake. FIG. 1G: N-acetylcysteine (NAC) does not affect the stability of the GFP^{degron} reporter, as measured by FACS.

FIG. 2. The figure highlights residues that are in close contact to EN106 and would be expected to impart at least some specificity (FEM1B—His185, Cys186, Gly187, Gly217, Asn216, His218, Asn340, and Ile341).

FIG. 3. Compound titration data and chemical structures for compounds EN-106 and EN-302.

FIG. 4. Western Blot showing degradation of BRD4 in 231MFP breast cancer cells by EN106 linked to BRD4 inhibitor JQ1 and by known Brd4 PROTAC MZ1, and the chemical structure of EN106 linked to BRD4 inhibitor JQ1.

FIG. 5. Loss of FEM1B increases muscle differentiation. C2C12 myoblasts were depleted of KEAP1 or FEM1B, and myotube formation was determined. Bottom: quantification of at least three biolocial replicates with mean SD.

FIGS. 6A-6C. Loss of FEM1B rescues muscle differentiation during stress. FIG. 6A: C2C12 cells were depleted of KEAP1, FEM1B, or both, and differentiation was analyzed by microscopy against MyHC. Bottom: quantification of four biolocial replicates with mean f SD. FIG. 6B: C2C12 cells were depleted of KEAP1, FEM1B, or both, and differentiation was analyzed by western blotting. FIG. 6C: C2C12 myoblasts depleted of FEM1B were treated throughout differentiation with the ROS scavengers S1QEL1.1 and S3QEL2. Bottom: quantification of three biolocial replicates with mean SD.

FIG. 7. Loss of FEM1B causes inactivation of the oncogenic transcription factor NRF2. qRT-PCR analysis of NRF2 target genes (GCLM, TALDO1, NQO, and HMOX1) or myogenesis markers (MYOG and MYL1) in C2C12 myoblasts depleted of KEAP1, FEM1B, or both. Quantifiation of three technical replicates SD.

FIG. 8. Loss of FEM1B causes nuclear exclusion of NRF2. NRF2 localization was determined by immunofluorescence in C2C12 myoblasts depleted of KEAP1, FEM1B, or both.

FIG. 9. FEM1B-C186 is required for substrate binding of FEM1B (e.g., FNIP1 recruitment). ^{FLAG}FEM1B, ^{FLAG}FEM1B^{C186S}, or ^{FLAG}FEM1B^{L597A} was purified from 293T cells that expressed hemagglutinin (HA)-tagged GATOR1 or FNIP1-FLCN subunits. Co-purifying proteins were detected by αHA-western blotting.

FIGS. 10A-10E. Loss of FEM1B shuts off mitochondria. FIG. 10A: C2C12 myoblasts were depleted of FEM1B, FNIP1, or both, and processed for transmission electron microscopy. FIG. 10B: C2C12 myoblasts were depleted of FEM1B, FNIP1, or both; incubated with the mitochontrial membrane potential dye TMRM; and analyzed by flow cytometry (control: CCCP-treated cells). FIG. 10C: Mitochondrial morphology was examined in C2C12 myoblasts depleted of FNIP1, FEM1B, or both by immunofluorescence microscopy against TOMM20. The distance of mitochondria from the nucleus was quantified (n=10-15 per condition). FIG. 10D: C2C12 myotubes were depleted of FEM1B, FNIP1, or both, and stained for mitochondrial superoxide using MitoSox. FIG. 10E: Model of the reductive stress response. Reductive stress reverses the oxidation of invariant Cys residues in the FNIP1 degron, leading to recofnition of FNIP1 by CUL2^{FEM1B}, polyubiquitylation, and proteasomal degradation. Loss of FNIP1 increases mitochondrial activity and triggers production of ROS to counteract reductive stress.

FIG. 11. Fluorescence polarization screen to identify functional FEM1B-FNIP1-targeting covalent ligands.

FIGS. 12A-12B. EN106 covalently targets C186 on FEM1B. FIG. 12A: Gel-based ABPP of EN106 against FEM1B. FIG. 12B: EN106 targets C186 in FEM1B. C186 is critical for substrate recognition of FEM1B. Sequence shown is: KAHCGATALHFAAEAGHIDIVKE (residues 183-205 of SEQ ID NO:1).

FIG. 13. EN10 inhibits endogenous and overexpressed FEM1B in cells and stabilizes FNIP1. HEK293T cells were transfected 24 hours before flow cytometery with the GFP-Fnip1 degron reporter+/−Fem1b. Cells were treated with DMSO or EN-106 (20 μM) for 8 hours.

FIGS. 14A-14C. EN106 engages FEM1B in cells. FIG. 14A: Structures of EN106 and EN106-alkyne (NJH-2-030). FIG. 14B: EN106-alkyne pulldown. See Example 4 for experimental procedure. FIG. 14C: HEK293T cells were treated with vehicle or EN106 (10 μM) for 2 h and cell lysates were subjected to the isoTOP-ABPP protocol described in Example 4.

FIGS. 15A-15F. Discovery of FEM1B recruiter for targeted protein degradation. FIG. 15A: Structure of NJH-01-106. FIG. 15B: Dose Response. HEK293T cells were treated with DMSO or NJH-01-106 at 10, 1, 0.1, or 0.01 μM for 8 hours. Cells were then harvested, lysed, and BRD4 abundance assessed by western blot. FIG. 15C: Proteasome inhibitor rescue. HEK293T cells were pretreated with DMSO or 1 M bortezomib for two hours before addition of DMSO or 10 M NJH-01-106 and incubation for 8 hours. Cells were then harvested, lysed, and BRD4 abundance assessed by western blot. FIG. 15D: Neddylation inhibitor rescue. HEK293T cells were pretreated with DMSO or 200 nM MLN4924 for two hours before addition of DMSO or 10 M NJH-01-106 and incubation for 8 hours. Cells were then harvested, lysed, and BRD4 abundance assessed by western blot. FIG. 15E: EN106 and nimbolide competition rescue. HEK293T cells were pretreated with either DMSO, 50 M EN106, or 1 M nimbolide for 2 hours. DMSO or 10 μM NJH-01-106 was then added before incubation for an additional 8 h. Cells were then harvested, lysed, and BRD4 abundance assessed by western blot. FIG. 15F: FEM1B KO rescue protocol. WT or FEM1B^{KO} HEK293T cells were treated with either DMSO or 1 M NJH-01-106 for 8 h. Cells were harvested, lysed with RIPA lysis buffer, protein concentration normalized, and protein abundance assessed by western blot.

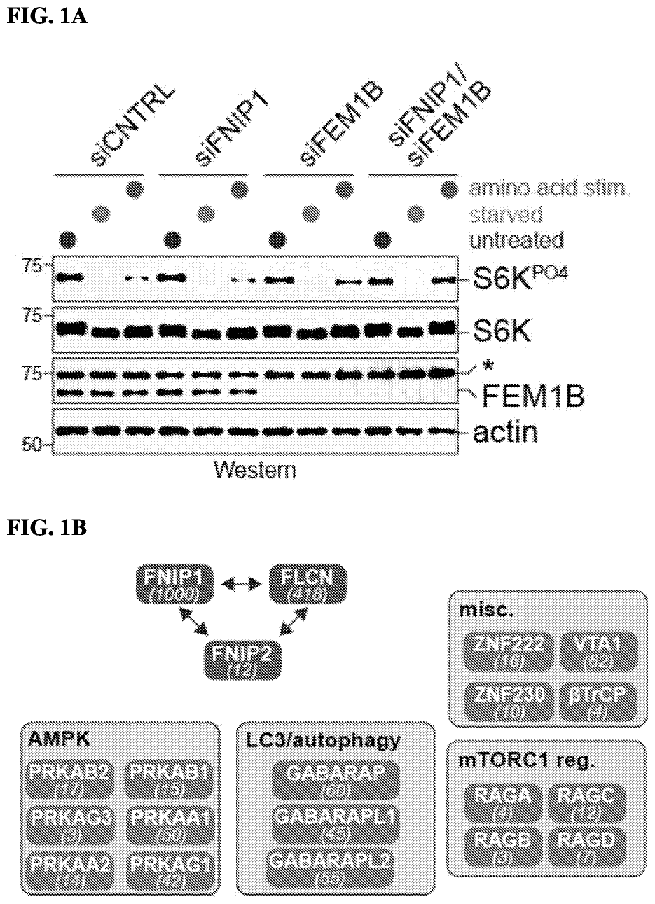

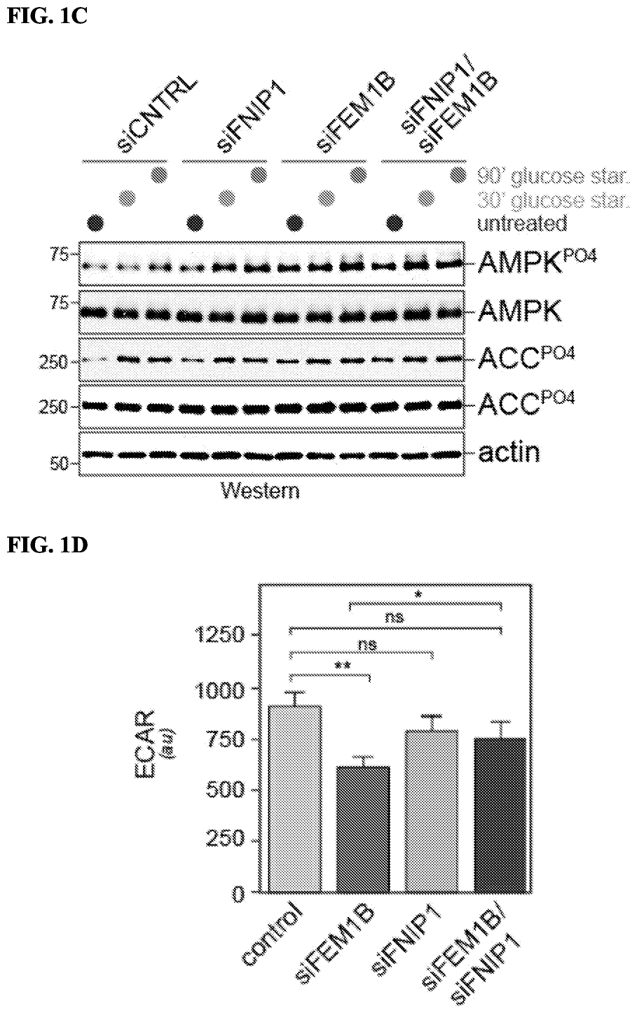

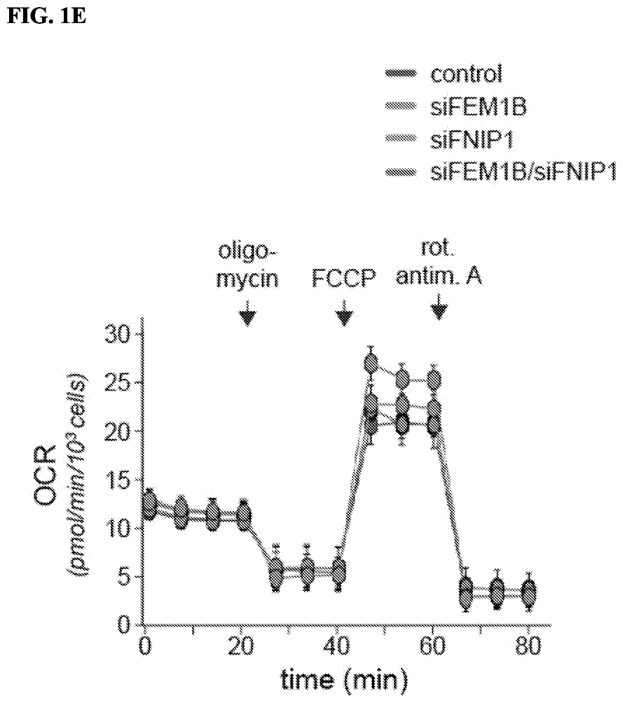

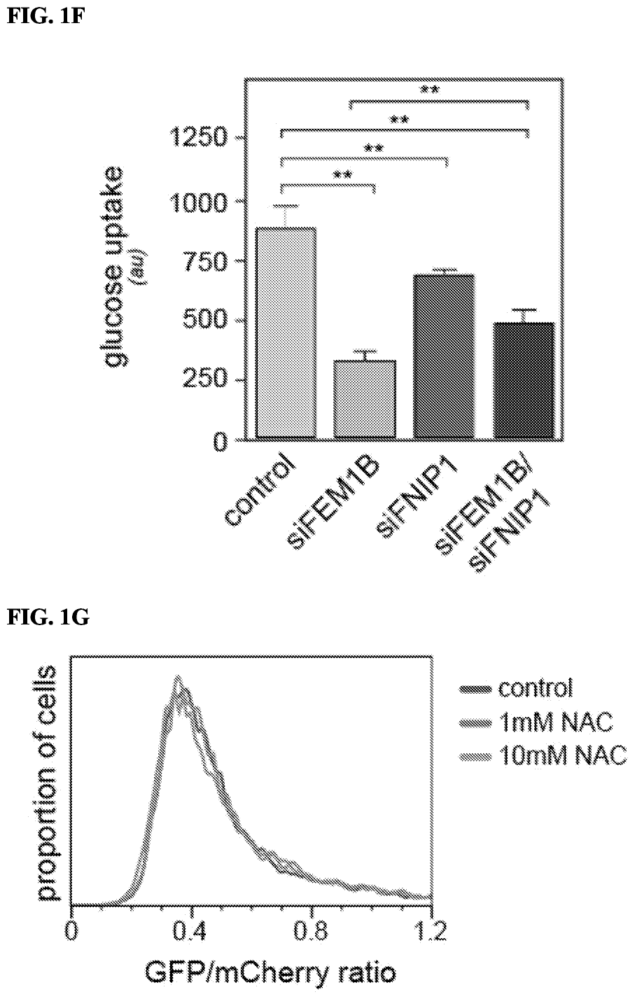

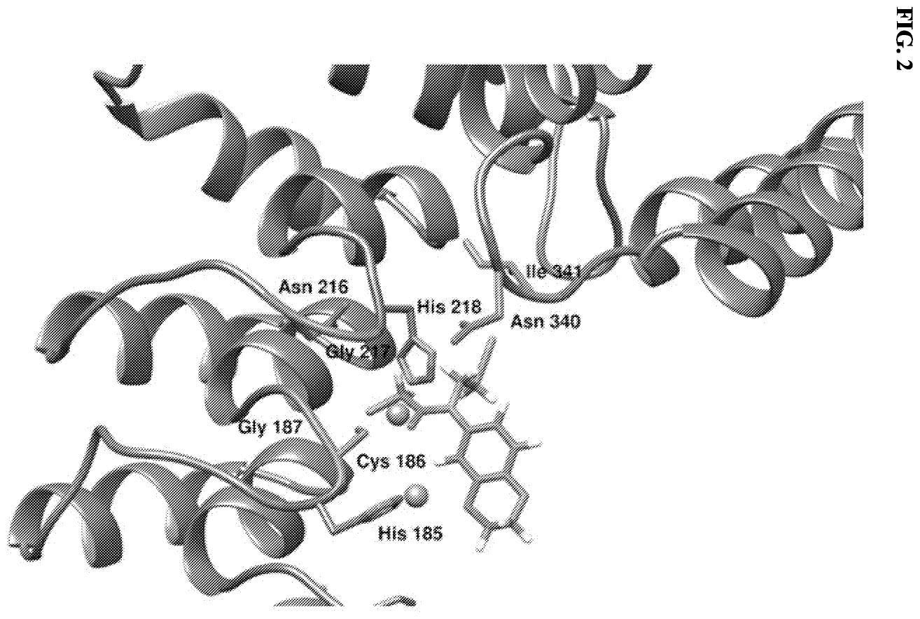

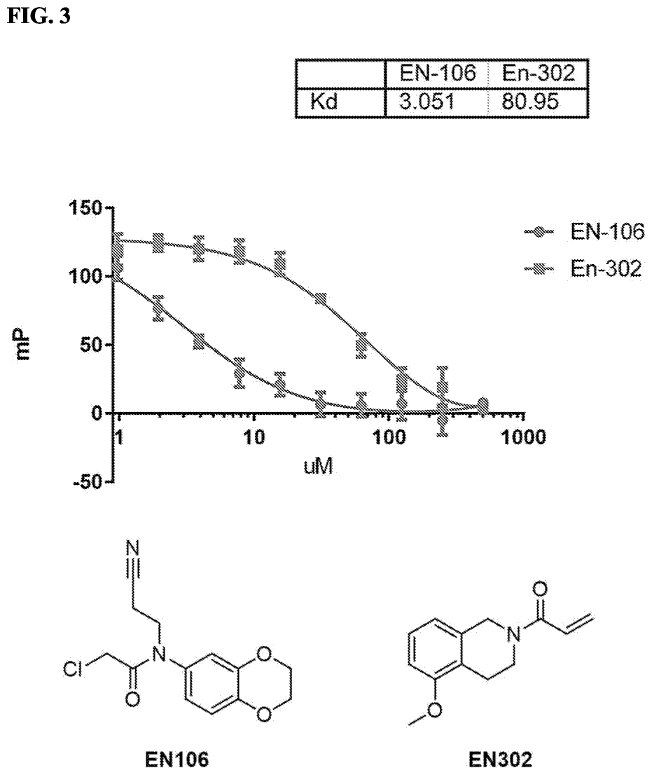

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings. In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dlhydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

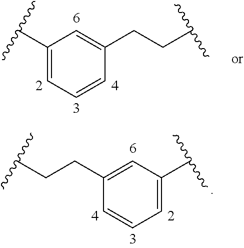 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SIR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″ ″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′R″″, —CN, —NO₂, —R′, —N₃, —CH(Ph)₂, fluoro(C₁-C₄)alkoxy, and fluoro(C₁-C₄alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —C3, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —C₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₆ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

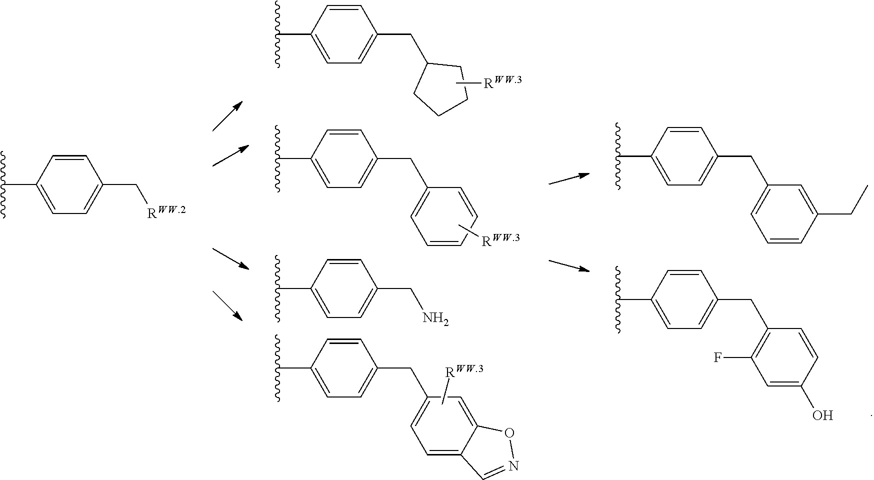

R^{WW.1} is independently oxo, halogen, —CX^{WW.1}₃, —CX^{WW.1}₂, —CH₂X^{WW.1}, —OCX^{WW.1}₃, —OCH₂X^{WW.1}, —OCHX^{WW.1}₂, —CN, —OH, —NH₂, —COH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, S to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₄, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100A.2}, and R^{WW.3} is R^{100B.3}, R^{WW.1}, R^{WW.2} and R^{WW.3} in this paragraph are as defined in the preceding paragraphs.

R^{LWW.1} is independently oxo, halogen, —CX^{LWW.1}₃, —CH^{LWW.1}₂, —CH₂X^{LWW.1}, —OCX^{LWW.1}₃, —OCH₂X^{LWW.1}, —OCH^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅—C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃—, —OCH₂X^{LWW.1}, —CN, —OH, NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OC₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂- —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g., phosphines) to form, for example, phosphate diester bonds;
(n) azides coupled to alkynes using copper catalyzed cycloaddition click chemistry, and
(o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

The term “covalent cysteine modifier moiety” as used herein refers to a monovalent electrophilic moiety that is able to measurably bind to a cysteine amino acid. In embodiments, the covalent cysteine modifier moiety binds via an irreversible covalent bond.

In embodiments, the covalent cysteine modifier moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

A “detectable agent” or “detectable moiety” is a composition, substance, element, or compound; or moiety thereof, detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁷Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{94m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perfiexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁰⁹Pm, ¹⁵³Sm, ¹⁵⁴⁻¹⁵³¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29,42,43,44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e., electronegative) or is deficient in electron density (i.e., electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g., a first linker or second linker), or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

The term “capable of binding” as used herein refers to a moiety (e.g., a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

The term “non-nucleophilic base” as used herein refers to any sterically hindered base that is a poor nucleophile.

The term “nucleophile” as used herein refers to a chemical species that donates an electron pair to an electrophile to form a chemical bond in relation to a reaction. All molecules or ions with a free pair of electrons or at least one pi bond can act as nucleophiles.

The term “strong acid” as used herein refers to an acid that is completely dissociated or ionized in an aqueous solution. Examples of common strong acids include hydrochloric acid (HCl), nitric acid (HNO₃), sulfuric acid (H₂SO₄), hydrobromic acid (HBr), hydroiodic acid (HI), perchloric acid (HClO₄), or chloric acid (HClO₃).

The term “carbocation stabilizing solvent” as used herein refers to any polar protic solvent capable of forming dipole-dipole interactions with a carbocation, thereby stabilizing the carbocation.

The term “target protein binding moiety” refers to a portion of a compound, as set forth herein, that is capable of binding to a target protein. In embodiments, the target protein binding moiety is a monovalent form of a ligand of a target protein. In embodiments, the target is a Brd4 protein. In embodiments, the target is a K-ras protein. In embodiments, the target is a Bruton's tyrosine kinase (BTK) protein. In embodiments, the target is an androgen receptor (AR) protein. In embodiments, the target is a MYC protein. In embodiments, the target is an N-MYC protein. In embodiments, the target is a beta-catenin protein. In embodiments, the target is a huntingtin (HTT) protein.

In embodiments, the Brd4 binding moiety binds to Brd4 with a Kd of less than one micromolar. In embodiments, the Brd4 binding moiety binds to Brd4 with a Kd of less than 500 nanomolar. In embodiments, the Brd4 binding moiety binds to Brd4 with a Kd of less than 450 nanomolar. In embodiments, the Brd4 binding moiety binds to Brd4 with a Kd of less than 400 nanomolar. In embodiments, the Brd4 binding moiety binds to Brd4 with a Kd of less than 350 nanomolar. In embodiments, the Brd4 binding moiety binds to Brd4 with a Kd of less than 300 nanomolar. In embodiments, the Brd4 binding moiety binds to Brd4with a Kd of less than 250 nanomolar. In embodiments, the Brd4 binding moiety binds to Brd4 with a Kd of less than 200 nanomolar. In embodiments, the Brd4 binding moiety binds to Brd4 with a Kd of less than 180 nanomolar. In embodiments, the Brd4 binding moiety binds to Brd4 with a Kd of less than 150 nanomolar. In embodiments, the Brd4 binding moiety binds to Brd4 with a Kd of less than 100 nanomolar. In embodiments, the Brd4 binding moiety binds to Brd4 with a Kd of less than 50 nanomolar. In embodiments, the Brd4 binding moiety binds to Brd4 with a Kd of less than 25 nanomolar. In embodiments, the Brd4 binding moiety binds to Brd4 with a Kd of less than 15 nanomolar. In embodiments, the Brd4 binding moiety binds to Brd4 with a Kd of less than 10 nanomolar. In embodiments, the Brd4 binding moiety binds to Brd4 with a Kd of less than 5 nanomolar. In embodiments, the Brd4 binding moiety binds to Brd4 with a Kd of less than one nanomolar. In embodiments, the Brd4 binding moiety binds to Brd4 with a Kd between 180 nM and 15 nM.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “EC50” or “half maximal effective concentration” as used herein refers to the concentration of a molecule (e.g., an LKB1 activator) capable of inducing a response which is halfway between the baseline response and the maximum response after a specified exposure time. In embodiments, the EC50 is the concentration of a molecule (e.g., an LKB1 activator) that produces 50% of the maximal possible effect of that molecule.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. The disease may be an autoimmune disease. The disease may be a neurodegenerative disease. The disease may be diabetes. The disease may be an inflammatory disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g., humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g., an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

An amino acid residue in a protein “corresponds” to a given residue when it occupies the same essential structural position within the protein as the given residue. For example, a selected residue in a selected protein corresponds to C185 of FEM1B protein when the selected residue occupies the same essential spatial or other structural relationship as C185 in FEM1B protein. In some embodiments, where a selected protein is aligned for maximum homology with the FEM1B protein, the position in the aligned selected protein aligning with C185 is said to correspond to C₁₈₅. Instead of a primary sequence alignment, a three dimensional structural alignment can also be used, e.g., where the structure of the selected protein is aligned for maximum correspondence with the FEM1B protein and the overall structures compared. In this case, an amino acid that occupies the same essential position as C185 in the structural model is said to correspond to the C185 residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue.

Instead of a primary sequence alignment, a three dimensional structural alignment can also be used, e.g., where the structure of the selected protein is aligned for maximum correspondence with the human protein and the overall structures compared. In this case, an amino acid that occupies the same essential position as a specified amino acid in the structural model is said to correspond to the specified residue. For example, a selected residue in a selected protein corresponds to C186 of a FEM1B protein (e.g., a human FEM1B protein) when the selected residue occupies the same essential spatial or other structural relationship as C186 in a FEM1B protein (e.g., a human FEM1B protein). In some embodiments, where a selected protein is aligned for maximum homology with the FEM1B protein, the position in the aligned selected protein aligning with C186 is said to correspond to C186 of the FEM1B protein (e.g., a human FEM1B protein). Instead of a primary sequence alignment, a three dimensional structural alignment can also be used, e.g., where the structure of the selected protein is aligned for maximum correspondence with the FEM1B protein (e.g., of SEQ ID NO:1) and the overall structures compared. In this case, an amino acid that occupies the same essential position as C186 of a FEM1B protein (e.g., a human FEM1B protein) in the structural model is said to correspond to the C186 residue. Another example is wherein a selected residue in a selected protein corresponds to C186 in a FEM1B protein (e.g., a human FEM1B protein) when the selected residue (e.g., cysteine residue) occupies essential the same sequence, spatial, or other structural position within the protein as C186 in the FEM1B protein (e.g., a human FEM1B protein).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

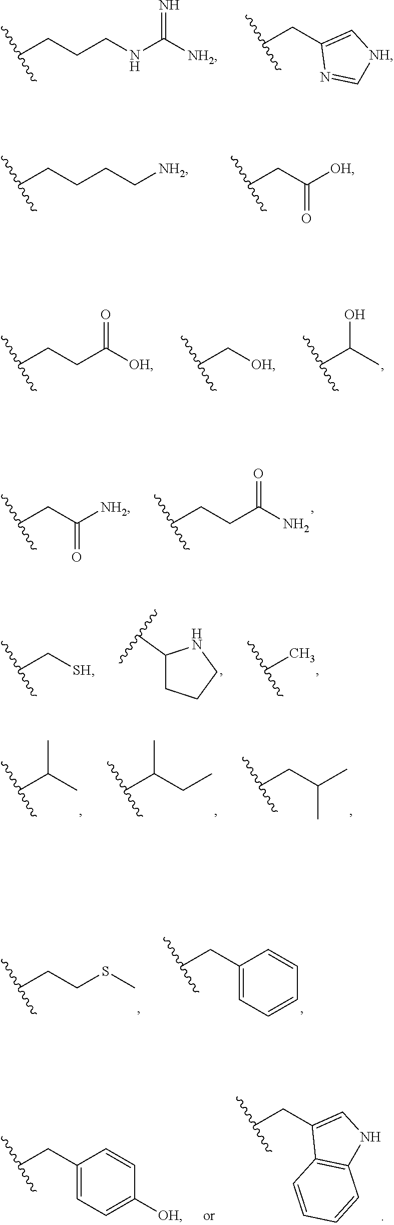

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafiuoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-p-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

### II. Compounds

In an aspect, provided herein is a compound having the formula:

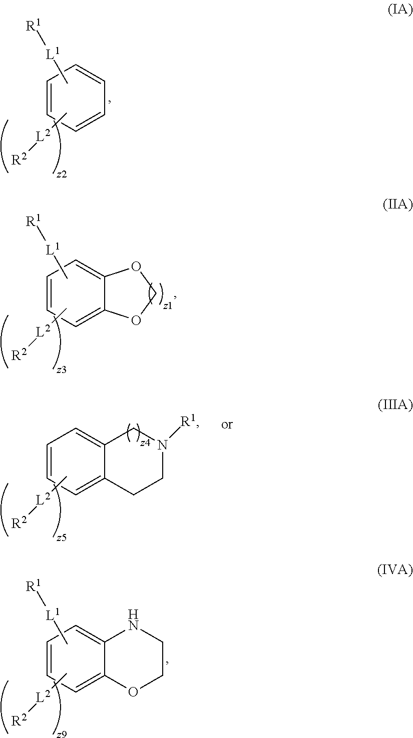

or a pharmaceutically acceptable salt thereof.

R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is an electrophilic moiety.

The symbol z1 is 1 or 2.

The symbol z2 is 0 to 5.

The symbol z3 is 0 to 3.

The symbol z4 is 0 or 1.

The symbols z5 and z9 are each independently an integer from 0 to 4.

In an aspect, provided herein is a compound having the formula:

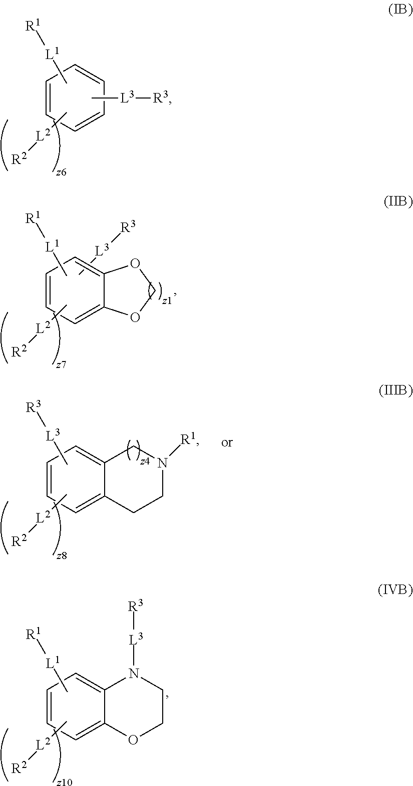

or a pharmaceutically acceptable salt thereof.

R², L², R¹⁰², L¹, R¹⁰¹, R¹, z1, and z4 are as described herein, including in embodiments.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}.

R¹⁰ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R³ is a target protein binding moiety.

The symbol z1 is 1 or 2.

The symbol z4 is 0 or 1.

The symbol z6 is 0 to 4.

The symbol z7 is 0 to 2.

The symbols z8 and z10 are each independently an integer from 0 to 3.

In embodiments, R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R³, L³, R¹⁰³, L^{3A}, L^{3B}, L^{3C}, L², R¹⁰², L¹, R¹⁰¹, R¹, z1, z2, z3, z4, z5, z6, z7, z8, z9, and z10 are as described herein, including embodiments.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is independently halogen, —CCl₃, —CBr₃, —CF₃, —CI₃, —CN, —OH, —NH₂, —COOH, substituted or unsubstituted alkyl, or substituted or unsubstituted heteroalkyl.

In embodiments, R² is independently halogen, —CCl₃, —CBr₃, —CF₃, —CI₃, —CN, —OH, —NH₂, —COOH, substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R² is independently halogen, —CCl₃, —CBr₃, —CF₃, —CI₃, —CN, —OH, —NH₂, or —COOH. In embodiments, R² is independently halogen. In embodiments, R² is independently —CCl₃. In embodiments, R² is independently —CBr₃. In embodiments, R² is independently —CF₃. In embodiments, R² is independently —CI₃. In embodiments, R² is independently —CN. In embodiments, R² is independently —OH. In embodiments, R² is independently —NH₂. In embodiments, R² is independently —COOH.

In embodiments, R² is independently substituted C₁-C₆ alkyl. In embodiments, R² is independently unsubstituted C₁-C₆ alkyl. In embodiments, R² is independently substituted 2 to 6 membered heteroalkyl. In embodiments, R² is independently unsubstituted 2 to 6 membered heteroalkyl.

In embodiments, R² is independently substituted C₁-C₃ alkyl. In embodiments, R² is independently unsubstituted C₁-C₃ alkyl. In embodiments, R² is independently substituted 2 to 3 membered heteroalkyl. In embodiments, R² is independently unsubstituted 2 to 3 membered heteroalkyl.

In embodiments, R² is independently oxo-substituted C₁-C₃ alkyl. In embodiments, R² is independently oxo-substituted C₁-C₃ alkyl. In embodiments, R² is independently —C(O)CH₃. In embodiments, R² is independently oxo-substituted C₁-C₈ alkynyl. In embodiments, R² is independently

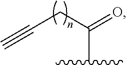

wherein n is an integer from 1 to 8. In embodiments, R² is independently

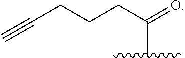 [SMILES: *C(=O)CCCC#C]

In embodiments, R² is independently oxo-substituted 2 to 8 membered heteroalkyl. In embodiments, R² is independently —C(O)OC(CH₃)₃. In embodiments, R² is independently -(unsubstituted C₁-C₄ alkyl)-C(O)OC(CH₃)₃. In embodiments, R² is independently —CH₂C(O)OC(CH₃)₃.

In embodiments, R² is independently halogen, —CF₃, unsubstituted C₁-C₃ alkyl or unsubstituted 2 to 3 membered heteroalkyl.

In embodiments, R² is independently —Cl, —Br, —F, —CF₃, —CH₃, —OCH₃, or —OCH₂CH₃. In embodiments, R² is independently —Cl. In embodiments, R² is independently —Br. In embodiments, R² is independently —F. In embodiments, R² is independently —CH₃.

In embodiments, R² is independently —OCH₃. In embodiments, R² is independently —OCH₂CH₃.

In embodiments, R¹ is independently an electrophilic moiety. In embodiments, R¹ is independently a covalent cysteine modifier moiety. In embodiments, R¹ is independently

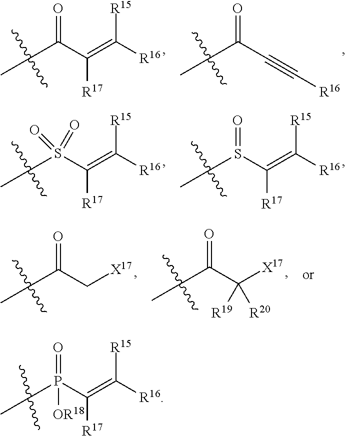

R³, L³, R¹⁰³, L^{3A}, L^{3B}, L^{3C}, R², L², R¹⁰², L¹, R¹⁰¹, R¹, z1, z2, z3, z4, z5, z6, z7, z8, z9, and z10 are as described herein, including embodiments.

R¹⁵, R¹⁶, R¹⁷, R¹⁸, R¹⁹, and R²⁰ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹⁷ is halogen.

In embodiments, R¹ is independently

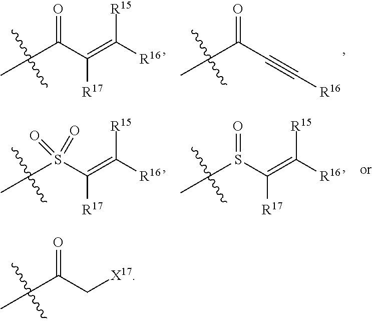

In embodiments, R¹ is independently

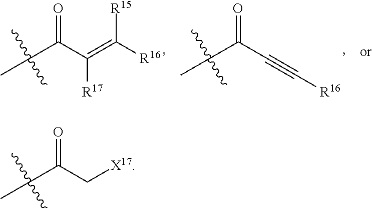

In embodiments, R¹ is independently

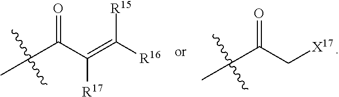

In embodiments, R¹ is independently

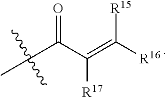

In embodiments, R¹ is independently

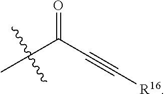

In embodiments, R¹ is independently

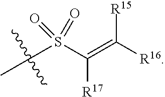

In embodiments, R¹ is independently

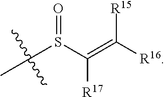

In embodiments, R¹ is independently

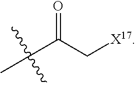

In embodiments, R¹ is independently

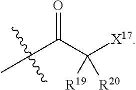

In embodiments, R¹ is independently

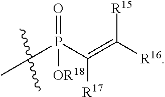

R³, L³, R¹⁰³, L^{3A}, L^{3B}, L^{3C}, R², L², R¹⁰², L¹, R¹⁰¹, z1, z2, z3, z4, z5, z6, z7, z8, z9, and z10 are as described herein, including embodiments.

In embodiments, R¹⁵, R¹⁶, R¹⁷, R¹⁸, R¹⁹, and R²⁰ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₄ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R³, L³, R¹⁰³, L^{3A}, L^{3B}, L^{3C}, R², L², R¹⁰², L¹, R¹⁰¹, R¹, z1, z2, z3, z4, z5, z6, z7, z8, z9, and z10 are as described herein, including embodiments.

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, a substituted R¹⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁸ is substituted with a plurality of groups selected from substituent groups, size-limited substituent groups, and lower substituent groups; each substituent group, size-limited substituent group, and/or lower substituent group may optionally be different. In embodiments, when R¹⁸ is substituted, it is substituted with at least one substituent group. In embodiments, when R¹⁸ is substituted, it is substituted with at least one size-limited substituent group. In embodiments, when R¹⁸ is substituted, it is substituted with at least one lower substituent group.

In embodiments, a substituted R¹⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁹ is substituted with a plurality of groups selected from substituent groups, size-limited substituent groups, and lower substituent groups; each substituent group, size-limited substituent group, and/or lower substituent group may optionally be different. In embodiments, when R¹⁹ is substituted, it is substituted with at least one substituent group. In embodiments, when R¹⁹ is substituted, it is substituted with at least one size-limited substituent group. In embodiments, when R¹⁹ is substituted, it is substituted with at least one lower substituent group.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, X¹⁷ is —F, —Cl, —I, or —Br. In embodiments, X¹⁷ is —F. In embodiments, X¹⁷ is —Cl. In embodiments, X¹⁷ is —I. In embodiments, X¹⁷ is —Br.

In embodiments, R¹⁵, R¹⁶, R¹⁷, R¹⁸, R¹⁹, and R²⁰ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, or —COOH. In embodiments, R¹⁵, R¹⁶, R¹⁷, R¹⁸, R¹⁹, and R²⁰ are independently hydrogen. In embodiments, R¹⁵, R¹⁶, R¹⁷, R¹⁸, R¹⁹, and R²⁰ are independently halogen. In embodiments, R¹⁵, R¹⁶, R¹⁷, R¹⁸, R¹⁹, and R²⁰ are independently —CCl₃. In embodiments, R¹⁵, R¹⁶, R¹⁷, R¹⁸, R¹⁹, and R²⁰ are independently —CBr₃. In embodiments, R¹⁵, R¹⁶, R¹⁷, R¹⁸, R¹⁹, and R²⁰ are independently —CF₃. In embodiments, R¹⁵, R¹⁶, R¹⁷, R¹⁸, R¹⁹, and R²⁰ are independently —CI₃. In embodiments, R¹⁵, R¹⁶, R¹⁷, R¹⁸, R¹⁹, and R²⁰ are independently —CHCl₂. In embodiments, R¹⁵, R¹⁶, R¹⁷, R¹⁸, R¹⁹, and R²⁰ are independently —CHBr₂. In embodiments, R¹⁵, R¹⁶, R¹⁷, R¹⁸, R¹⁹, and R²⁰ are independently —CHF₂. In embodiments, R¹⁵, R¹⁶, R¹⁷, R¹⁸, R¹⁹, and R²⁰ are independently —CHI₂. In embodiments, R¹⁵, R¹⁶, R¹⁷, R¹⁸, R¹⁹, and R²⁰ are independently —CH₂Cl. In embodiments, R¹⁵, R¹⁶, R¹⁷, R¹⁸, R¹⁹, and R²⁰ are independently —CH₂Br. In embodiments, R¹⁵, R¹⁶, R¹⁷, R¹⁸, R¹⁹, and R²⁰ are independently —CH₂F. In embodiments, R¹⁵, R¹⁶, R¹⁷, R¹⁸, R¹⁹, and R²⁰ are independently —CH₂I. In embodiments, R¹⁵, R¹⁶, R¹⁷, R¹⁸, R¹⁹, and R²⁰ are independently —CN. In embodiments, R¹⁵, R¹⁶, R¹⁷, R¹⁸, R¹⁹, and R²⁰ are independently —OH. In embodiments, R¹⁵, R¹⁶, R¹⁷, R¹⁸, R¹⁹, and R²⁰ are independently —NH₂. In embodiments, R¹⁵, R¹⁶, R¹⁷, R¹⁸, R¹⁹, and R²⁰ are independently —COOH.

In embodiments, R¹⁵, R¹⁶, R¹⁷, R¹⁸, R¹⁹, and R²⁰ are independently substituted or unsubstituted C₁-C₆ alkyl. In embodiments, R¹⁵, R¹⁶, R¹⁷, R¹⁸, R¹⁹, and R²⁰ are independently substituted or unsubstituted 2 to 6 membered heteroalkyl. In embodiments, R¹⁵, R¹⁶, R¹⁷, R¹⁸, R¹⁹, and R²⁰ are independently substituted or unsubstituted C₃-C₆ cycloalkyl. In embodiments, R¹⁵, R¹⁶, R¹⁷, R¹⁸, R¹⁹, and R⁰ are independently substituted or unsubstituted 3 to 6 membered heterocycloalkyl. In embodiments, R¹⁵, R¹⁶, R¹⁷, R¹⁸, R¹⁹, and R⁰ are independently substituted or unsubstituted C₆-C₁₀ aryl. In embodiments, R¹⁵, R¹⁶, R¹⁷, R¹⁸, R¹⁹, and R²⁰ are independently substituted or unsubstituted 5 to 10 membered heteroaryl.

In embodiments, R¹⁵, R¹⁶, R¹⁷, R¹⁸, R¹⁹, and R²⁰ are independently substituted C₁-C₆ alkyl. In embodiments, R¹⁵, R¹⁶, R¹⁷, R¹⁸, R¹⁹, and R²⁰ are independently unsubstituted C₁-C₆ alkyl. In embodiments, R¹⁵, R¹⁶, R¹⁷, R¹⁸, R¹⁹, and R²⁰ are independently substituted 2 to 6 membered heteroalkyl. In embodiments, R¹⁵, R¹⁶, R¹⁷, R¹⁸, R¹⁹, and R²⁰ are independently unsubstituted 2 to 6 membered heteroalkyl. In embodiments, R¹⁵, R¹⁶, R¹⁷, R¹⁸, R¹⁹, and R²⁰ are independently substituted C₃-C₆ cycloalkyl. In embodiments, R¹⁵, R¹⁶, R¹⁷, R¹⁸, R¹⁹, and R²⁰ are independently unsubstituted C₃-C₆ cycloalkyl. In embodiments, R¹⁵, R¹⁶, R¹⁷, R¹⁸, R¹⁹, and R²⁰ are independently substituted 3 to 6 membered heterocycloalkyl. In embodiments, R¹⁵, R¹⁶, R¹⁷, R¹⁸, R¹⁹, and R²⁰ are independently substituted 3 to 6 membered heterocycloalkyl. In embodiments, R¹⁵, R¹⁶, R¹⁷, R¹⁸, R¹⁹, and R²⁰ are independently substituted C₆-C₁₀ aryl. In embodiments, R¹⁵, R¹⁶, R¹⁷, R¹⁸, R¹⁹, and R²⁰ are independently substituted C₆-C₁₀ aryl. In embodiments, R¹⁵, R¹⁶, R¹⁷, R¹⁸, R¹⁹, and R²⁰ are independently substituted 5 to 10 membered heteroaryl. In embodiments, R¹⁵, R¹⁶, R¹⁷, R¹⁸, R¹⁹, and R²⁰ are independently substituted 5 to 10 membered heteroaryl.

In embodiments, R¹⁵, R¹⁶, R¹⁷, R¹⁸, R¹⁹, and R²⁰ are independently hydrogen. In embodiments, R¹⁵, R¹⁶, R¹⁷, R¹⁸, R¹⁹, and R²⁰ are independently methyl. In embodiments, R¹⁵, R¹⁶, R¹⁷, R¹⁸, R¹⁹, and R²⁰ are independently unsubstituted methyl.

In embodiments, R¹ is independently

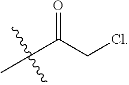 [SMILES: *C(=O)CCl]

In embodiments, R¹ is independently

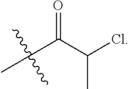 [SMILES: *C(=O)C(C)Cl]

In embodiments, R¹ is independently

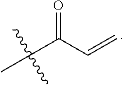 [SMILES: *C(=O)C=C]

In embodiments, L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O)—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene).

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O)—, —OC(O)—, substituted or unsubstituted alkylene, substituted or unsubstituted heteroalkylene. R¹⁵, R¹⁶, R¹⁷, R¹⁸, R¹⁹, R²⁰, R³, L³, R¹⁰³, L^{3A}, L^{3B}, L^{3C}, R², R¹⁰², L¹, R¹⁰¹, R¹, z1, z2, z3, z4, z5, z6, z7, z8, z9, and z10 are as described herein, including embodiments.

In embodiments, L² is independently a bond, —N(R¹⁰²)—, —C(O)—, substituted or unsubstituted alkylene, or substituted or unsubstituted heteroalkylene. In embodiments, L² is independently a bond, —N(R¹⁰²)—, —C(O)—, substituted or unsubstituted alkylene (e.g., C₁-C₈ alkylene, C₁-C₆ alkylene, or C₁-C₄ alkylene), or substituted or unsubstituted heteroalkylene (e.g., 2 to 8 membered heteroalkylene, 2 to 6 membered heteroalkylene, or 2 to 4 membered heteroalkylene).

In embodiments, L² is independently a bond. In embodiments, L² is independently —S(O)₂—. In embodiments, L² is independently —N(R¹⁰²)—. In embodiments, L² is independently —O—. In embodiments, L² is independently —S—. In embodiments, L² is independently —C(O)—. In embodiments, L² is independently —C(O)N(R¹⁰²)—. In embodiments, L² is independently —N(R¹⁰²)C(O)—. In embodiments, L² is independently —N(R¹⁰²)C(O)NH—. In embodiments, L² is independently —NHC(O)N(R¹⁰²)—. In embodiments, L² is independently —C(O)O—. In embodiments, L² is independently —OC(O)—. In embodiments, L² is independently substituted alkylene. In embodiments, L² is independently unsubstituted alkylene. In embodiments, L² is independently substituted heteroalkylene. In embodiments, L² is independently unsubstituted heteroalkylene.

In embodiments, L² is independently substituted C₁-C⁶ alkylene. In embodiments, L² is independently unsubstituted C₁-C⁶ alkylene. In embodiments, L² is independently substituted 2 to 6 membered heteroalkylene. In embodiments, L² is independently unsubstituted 2 to 6 membered heteroalkylene.

In embodiments, L² is independently a bond.

In embodiments, R¹⁰² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R¹⁵, R¹⁶, R¹⁷, R¹⁸, R¹⁹, R²⁰, R³, L³, R¹⁰³, L^{3A}, L^{3B}, L^{3C}, R², L¹, R¹⁰¹, R¹, z1, z2, z3, z4, z5, z6, z7, z8, z9, and z10 are as described herein, including embodiments.

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently hydrogen, halogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R¹⁰² is independently hydrogen. In embodiments, R¹⁰² is independently halogen. In embodiments, R¹⁰ is substituted C₁-C₆ alkyl. In embodiments, R¹⁰² is unsubstituted C₁-C₆ alkyl. In embodiments, R¹⁰² is substituted 2 to 6 membered heteroalkyl. In embodiments, R¹⁰² is unsubstituted 2 to 6 membered heteroalkyl. In embodiments, R¹⁰² is substituted C₃-C₆ cycloalkyl. In embodiments, R¹⁰² is unsubstituted C₃-C₆ cycloalkyl. In embodiments, R¹⁰² is substituted 3 to 6 membered heterocycloalkyl. In embodiments, R¹⁰² is unsubstituted 3 to 6 membered heterocycloalkyl. In embodiments, R¹⁰² is substituted C₆-C₁₀ aryl. In embodiments, R¹⁰² is unsubstituted C₆-C₁₀ aryl. In embodiments, R¹⁰² is substituted 5 to 10 membered heteroaryl. In embodiments, R¹⁰² is unsubstituted 5 to 10 membered heteroaryl.

In embodiments, R¹⁰² is independently hydrogen or unsubstituted alkyl. In embodiments, R¹⁰² is independently hydrogen or unsubstituted C₁-C₆ alkyl. In embodiments, R¹⁰² is independently hydrogen. In embodiments, R¹⁰² is independently unsubstituted C₁-C₆ alkyl.

In embodiments, R¹⁰² is independently methyl, ethyl, propyl, or butyl. In embodiments, R¹⁰² is independently methyl. In embodiments, R¹⁰² is independently ethyl. In embodiments, R¹⁰² is independently propyl. In embodiments, R¹⁰² is independently butyl.

In embodiments, R¹⁰² is independently unsubstituted methyl, unsubstituted ethyl, unsubstituted propyl, or unsubstituted butyl. In embodiments, R¹⁰² is independently unsubstituted methyl. In embodiments, R¹⁰² is independently unsubstituted ethyl. In embodiments, R¹⁰² is independently unsubstituted propyl. In embodiments, R¹⁰² is independently unsubstituted butyl.

In embodiments, L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). R¹⁵, R¹⁶, R¹⁷, R¹⁸, R¹⁹, R²⁰, R³, L³, R¹⁰³, L^{3A}, L^{3B}, L^{3C}, R², R¹⁰², R¹⁰¹, R¹, z1, z2, z3, z4, z5, z6, z7, z8, z9, and z10 are as described herein, including embodiments.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—. In embodiments, L¹ is a bond. In embodiments, L¹ is —N(R¹⁰¹)—. In embodiments, L¹ is —O—. In embodiments, L¹ is —C(O)—. In embodiments, L¹ is —C(O)N(R¹⁰¹)—. In embodiments, L¹ is —N(R¹⁰¹)C(O)—. In embodiments, L¹ is —N(R¹⁰¹)C(O)NH—. In embodiments, L¹ is —NHC(O)N(R¹⁰¹)—.

In embodiments, L¹ is substituted C₁-C₆ alkylene. In embodiments, L¹ is unsubstituted C₁-C₆ alkylene. In embodiments, L¹ is substituted 2 to 6 membered heteroalkylene. In embodiments, L¹ is unsubstituted 2 to 6 membered heteroalkylene. In embodiments, L¹ is substituted C₃-C₆ cycloalkylene. In embodiments, L is unsubstituted C₃-C₆ cycloalkylene. In embodiments, L is substituted 3 to 6 membered heterocycloalkylene. In embodiments, L¹ is unsubstituted 3 to 6 membered heterocycloalkylene. In embodiments, L¹ is substituted C₆-C₁₀ arylene. In embodiments, L¹ is unsubstituted C₆-C₁₀ arylene. In embodiments, L¹ is substituted 5 to 10 membered heteroarylene. In embodiments, L¹ is unsubstituted 5 to 10 membered heteroarylene.

In embodiments, R¹⁰¹ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂,—OCH₂CL, —OCH₂Br, —OCH₂I, —OCH₂F, —N₃, —SF₅, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R¹⁵, R¹⁶, R¹⁷, R¹⁸, R¹⁹, R²⁰, R³, L³, R¹⁰³, L^{3A}, L^{3B}, L^{3C}, R², R¹⁰², L¹, R¹, z1, z2, z3, z4, z5, z6, z7, z8, z9, and z10 are as described herein, including embodiments.

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is independently hydrogen, —OH, —NH₂, —COOH, —CONH₂, substituted or unsubstituted alkyl, substituted or unsubstituted aryl, or substituted or unsubstituted heteroaryl. In embodiments, R¹⁰¹ is independently hydrogen, —OH, —NH₂, —COOH, —CONH₂, substituted or unsubstituted C₁-C₆ alkyl, substituted or unsubstituted C₆-C₁₀ aryl, or substituted or unsubstituted 5 to 10 membered heteroaryl.

In embodiments, R¹⁰¹ is independently hydrogen. In embodiments, R¹⁰¹ is independently —OH. In embodiments, R¹⁰¹ is independently —NH₂. In embodiments, R¹⁰¹ is independently —COOH. In embodiments, R¹⁰¹ is independently —CONH₂. In embodiments, R¹⁰¹ is substituted C₁-C₆ alkyl. In embodiments, R¹⁰¹ is unsubstituted C₁-C₆ alkyl. In embodiments, R¹⁰¹ is substituted 2 to 6 membered heteroalkyl. In embodiments, R¹⁰¹ is unsubstituted 2 to 6 membered heteroalkyl. In embodiments, R¹⁰¹ is substituted C₃-C₆ cycloalkyl. In embodiments, R¹⁰¹ is unsubstituted C₃-C₆ cycloalkyl. In embodiments, R¹⁰¹ is substituted 3 to 6 membered heterocycloalkyl. In embodiments, R¹⁰¹ is unsubstituted 3 to 6 membered heterocycloalkyl. In embodiments, R¹⁰¹ is substituted C₆-C₁₀ aryl. In embodiments, R¹⁰¹ is unsubstituted C₆-C₁₀ aryl. In embodiments, R¹⁰¹ is substituted 5 to 10 membered heteroaryl. In embodiments, R¹⁰¹ is unsubstituted 5 to 10 membered heteroaryl.

In embodiments, R¹⁰¹ is independently hydrogen, substituted or unsubstituted alkyl, substituted or unsubstituted aryl, or substituted or unsubstituted heteroaryl. In embodiments, R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl.

In embodiments, R¹⁰¹ is independently hydrogen, —CH₂CH₂CN,

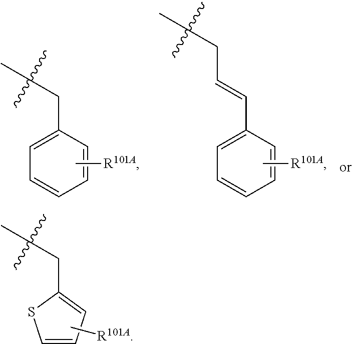

R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰¹ is independently hydrogen. In embodiments, R¹⁰¹ is independently —CH₂CH₂CN. In embodiments, R¹⁰¹ is independently

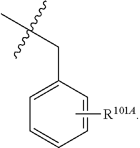

In embodiments, R¹⁰¹ is independently

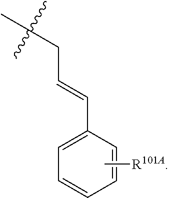

In embodiments, R¹⁰¹ is independently

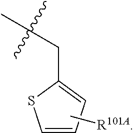

In embodiments, R^{101A} is independently hydrogen, halogen, —OH, —NH₂, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, a substituted R^{101A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{101A} is substituted with a plurality of groups selected from substituent groups, size-limited substituent groups, and lower substituent groups; each substituent group, size-limited substituent group, and/or lower substituent group may optionally be different. In embodiments, when R^{101A} is substituted, it is substituted with at least one substituent group. In embodiments, when R^{101A} is substituted, it is substituted with at least one size-limited substituent group. In embodiments, when R^{101A} is substituted, it is substituted with at least one lower substituent group.

In embodiments, R^{101A} is independently hydrogen or

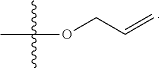 [SMILES: *OCC=C]

In embodiments, R^{101A} is independently hydrogen, —OCH₃, or

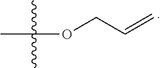 [SMILES: *OCC=C]

In embodiment R^{101A} is independently hydrogen. In embodiments, R^{101A} is independently

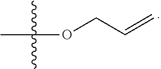 [SMILES: *OCC=C]

In embodiments, R^{101A} is independently halogen. In embodiments, R^{101A} is independently —OH. In embodiments, R^{101A} is independently —NH₂. In embodiments, R^{101A} is independently —COOH. In embodiments, R^{101A} is independently —CONH₂. In embodiments, R^{101A} is independently substituted C₁-C₆ alkyl. In embodiments, R^{101A} is independently unsubstituted C₁-C₆ alkyl. In embodiments, R^{101A} is independently substituted 2 to 6 membered heteroalkyl. In embodiments, R^{101A} is independently unsubstituted 2 to 6 membered heteroalkyl. In embodiments, R^{101A} is independently substituted or unsubstituted alkoxy. In embodiments, R^{101A} is independently unsubstituted alkoxy. In embodiments, R^{101A} is independently —O-(unsubstituted C₁-C₄ alkyl). In embodiments, R^{101A} is independently unsubstituted methoxy. In embodiments, R^{101A} is independently unsubstituted ethoxy. In embodiments, R^{101A} is independently unsubstituted propoxy. In embodiments, R^{101A} is independently unsubstituted n-propoxy. In embodiments, R^{101A} is independently unsubstituted isopropoxy. In embodiments, R^{101A} is independently unsubstituted butoxy. In embodiments, R^{101A} is independently unsubstituted n-butoxy. In embodiments, R^{101A} is independently unsubstituted tert-butoxy. In embodiments, R^{101A} is independently substituted C₃-C₆ cycloalkyl. In embodiments, R^{101A} is independently unsubstituted C₃-C₆ cycloalkyl. In embodiments, R^{101A} is independently substituted 3 to 6 membered heterocycloalkyl. In embodiments, R^{101A} is independently unsubstituted 3 to 6 membered heterocycloalkyl. In embodiments, R^{101A} is independently substituted C₆-C₁₀ aryl. In embodiments, R^{101A} is independently unsubstituted C₆-C₁₀ aryl. In embodiments, R^{101A} is independently substituted 5 to 10 membered heteroaryl. In embodiments, R^{101A} is independently unsubstituted 5 to 10 membered heteroaryl.

In embodiments, R¹⁰¹ is independently

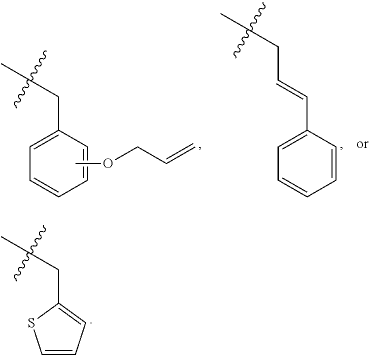

In embodiments, R¹⁰¹ is independently

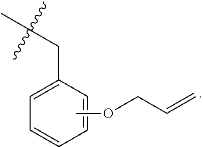

In embodiments, R¹⁰¹ is independently

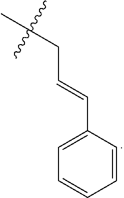 [SMILES: *C/C=C/c1ccccc1]

In embodiments, R¹⁰¹ is independently

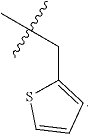 [SMILES: *Cc1cccs1]

In embodiments, R¹⁰ is independently

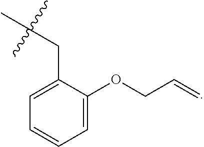

In embodiments, R¹⁰¹ is independently

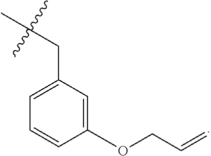

In embodiments, R¹⁰¹ is independently

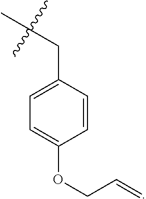

In embodiments, L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), substituted or unsubstituted heteroarylene (e.g., 5 to 10 membered heteroarylene, 5 to 9 membered heteroarylene, or 5 to 6 membered heteroarylene), or -L^{3A}-L^{3B}-L^{3C}-. L^{3A}, L^{3B}, L^{3C}, and R¹⁰³ are as described herein, including embodiments.

In embodiments, L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). R¹⁵, R¹⁶, R¹⁷, R¹⁸, R¹⁹, R²⁰, R³, R¹⁰², L^{3A}, L^{3B}, L^{3C}, R², R¹⁰², L¹, R¹⁰¹, R¹, z1, z2, z3, z4, z5, z6, z7, z8, z9, and z10 are as described herein, including embodiments.

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, —N(R¹⁰³)—, —O—, —S—, —C(O)—, substituted or unsubstituted alkylene, or substituted or unsubstituted heteroalkylene. In embodiments, L³ is a bond, —N(R¹⁰³)—, —O—, —S—, —C(O)—, substituted or unsubstituted alkylene (e.g., C₁-C₈ alkylene, C₁-C₆ alkylene, or C₁-C₄ alkylene), or substituted or unsubstituted heteroalkylene (e.g., 2 to 8 membered heteroalkylene, 2 to 6 membered heteroalkylene, or 2 to 4 membered heteroalkylene). In embodiments, L³ is a bond, —N(R¹⁰)—, —O—, —S—, —C(O)—, substituted or unsubstituted C₁-C₆ alkylene or substituted or unsubstituted 2 to 6 membered heteroalkylene.

In embodiments, L³ is a bond, substituted or unsubstituted alkylene, or substituted or unsubstituted heteroalkylene. In embodiments, L³ is a bond, substituted or unsubstituted C₁-C₆ alkylene, or substituted or unsubstituted 2 to 6 membered heteroalkylene. In embodiments, L³ is a bond or substituted or unsubstituted heteroalkylene. In embodiments, L³ is a bond or substituted or unsubstituted 2 to 6 membered heteroalkylene.

In embodiments, L³ is a bond. In embodiments, L³ is —N(R¹⁰³)—. In embodiments, L³ is —O—. In embodiments, L³ is —S—. In embodiments, L³ is —C(O)—. In embodiments, L³ is substituted or unsubstituted C₁-C₆ alkylene. In embodiments, L³ is substituted C₁-C₆ alkylene. In embodiments, L³ is unsubstituted C₁-C₆ alkylene. In embodiments, L³ is substituted or unsubstituted 2 to 6 membered heteroalkylene. In embodiments, L³ is substituted 2 to 6 membered heteroalkylene. In embodiments, L³ is unsubstituted 2 to 6 membered heteroalkylene.

In embodiments, L³ is

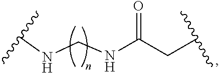

wherein n is an integer from 1 to 8. In embodiments, L³ is

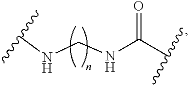

wherein n is an integer from 1 to 8. In embodiments, L³ is

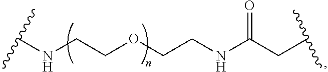

wherein n is an integer from 1 to 8. In embodiments, L³ is

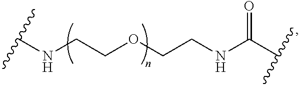

wherein n is an integer from 1 to 8.

In embodiments, L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene).

In embodiments, L^{3A} is a bond. In embodiments, L^{3A} is —NH—. In embodiments, L^{3A} is substituted or unsubstituted alkylene. In embodiments, L^{3A} is substituted or unsubstituted C₁-C₈ alkylene. In embodiments, L^{3A} is substituted C₁-C₈ alkylene. In embodiments, L^{3A} is oxo-substituted C₁-C₈ alkylene. In embodiments, L^{3A} is unsubstituted C₁-C₈ alkylene. In embodiments, L^{3A} is substituted or unsubstituted heteroalkylene. In embodiments, L^{3A} is substituted or unsubstituted 2 to 8 membered heteroalkylene. In embodiments, L^{3A} is substituted 2 to 8 membered heteroalkylene. In embodiments, L^{3A} is oxo-substituted 2 to 8 membered heteroalkylene. In embodiments, L^{3A} is unsubstituted 2 to 8 membered heteroalkylene.

In embodiments, a substituted L^{3A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{3A} is substituted with a plurality of groups selected from substituent groups, size-limited substituent groups, and lower substituent groups; each substituent group, size-limited substituent group, and/or lower substituent group may optionally be different. In embodiments, when L^{3A} is substituted, it is substituted with at least one substituent group. In embodiments, when L^{3A} is substituted, it is substituted with at least one size-limited substituent group. In embodiments, when L^{3A} is substituted, it is substituted with at least one lower substituent group.

In embodiments, L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene).

In embodiments, L^{3B} is a bond. In embodiments, L^{3B} is substituted or unsubstituted alkylene. In embodiments, L^{3B} is substituted or unsubstituted C₁-C₂₀ alkylene. In embodiments, L^{3B} is substituted or unsubstituted C₁-C₁₂ alkylene. In embodiments, L^{3B} is substituted or unsubstituted C₁-C₈ alkylene. In embodiments, L^{3B} is substituted or unsubstituted heteroalkylene. In embodiments, L^{3B} is substituted or unsubstituted 2 to 30 membered heteroalkylene. In embodiments, L^{3B} is substituted or unsubstituted 2 to 20 membered heteroalkylene. In embodiments, L^{3B} is substituted or unsubstituted 2 to 12 membered heteroalkylene. In embodiments, L^{3B} is substituted or unsubstituted 2 to 8 membered heteroalkylene. In embodiments, L^{3B} is an unsubstituted divalent form of polyethylene glycol. In embodiments, L^{3B} is

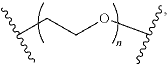

wherein n is an integer from 1 to 8.

In embodiments, a substituted L^{3B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{3B} is substituted with a plurality of groups selected from substituent groups, size-limited substituent groups, and lower substituent groups; each substituent group, size-limited substituent group, and/or lower substituent group may optionally be different. In embodiments, when L^{3B} is substituted, itis substituted with at least one substituent group. In embodiments, when L^{3B} is substituted, itis substituted with at least one size-limited substituent group. In embodiments, when L^{3B} is substituted, itis substituted with at least one lower substituent group.

In embodiments, Lac is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene).

In embodiments, L^{3C} is a bond. In embodiments, L^{3C} is —NH—. In embodiments, L^{3C} is —NHC(O)—. In embodiments, L^{3C} is —NHC(O)-(unsubstituted C₁-C₈ akylene)-. In embodiments, L^{3C} is —NHC(O)CH₂—. In embodiments, L^{3C} is substituted or unsubstituted alkylene. In embodiments, L^{3C} is substituted or unsubstituted C₁-C₈ alkylene. In embodiments, L^{3C} is substituted C₁-C₈ alkylene. In embodiments, L^{3C} is oxo-substituted C₁-C₈ alkylene. In embodiments, L^{3C} is unsubstituted C₁-C₈ alkylene. In embodiments, L^{3C} is substituted or unsubstituted heteroalkylene. In embodiments, L^{3C} is substituted or unsubstituted 2 to 8 membered heteroalkylene. In embodiments, L^{3C} is substituted 2 to 8 membered heteroalkylene. In embodiments, L^{3C} is oxo-substituted 2 to 8 membered heteroalkylene. In embodiments, L^{3C} is unsubstituted 2 to 8 membered heteroalkylene.

In embodiments, a substituted L^{3C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{3C} is substituted with a plurality of groups selected from substituent groups, size-limited substituent groups, and lower substituent groups; each substituent group, size-limited substituent group, and/or lower substituent group may optionally be different. In embodiments, when L^{3C} is substituted, it is substituted with at least one substituent group. In embodiments, when L^{3C} is substituted, it is substituted with at least one size-limited substituent group. In embodiments, when L^{3C} is substituted, it is substituted with at least one lower substituent group.

In embodiments, R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R¹⁵, R¹⁶, R¹⁷, R¹⁸, R¹⁹, R²⁰, R³, L³, L^{3A}, L^{3B}, L^{3C}, R², R¹⁰², L¹, R¹⁰¹, R¹, z1, z2, z3, z4, z5, z6, z7, z8, z9, and z10 are as described herein, including embodiments.

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is independently hydrogen, —OH, or substituted or unsubstituted alkyl. In embodiments, R¹⁰³ is independently hydrogen, —OH, or substituted or unsubstituted C₁-C₆ alkyl. In embodiments, R¹⁰³ is independently hydrogen. In embodiments, R¹⁰³ is independently —OH. In embodiments, R¹⁰³ is independently substituted or unsubstituted C₁-C₆ alkyl. In embodiments, R¹⁰³ is independently substituted C₁-C₆ alkyl. In embodiments, R¹⁰³ is independently unsubstituted C₁-C₆ alkyl.

In embodiments, R¹⁰³ is independently methyl. In embodiments, R¹⁰³ is independently ethyl. In embodiments, R¹⁰³ is independently propyl. In embodiments, R¹⁰³ is independently butyl.

In embodiments, R¹⁰³ is independently unsubstituted methyl. In embodiments, R¹⁰³ is independently unsubstituted ethyl. In embodiments, R¹⁰³ is independently unsubstituted propyl. In embodiments, R¹⁰³ is independently unsubstituted butyl.

In embodiments, R³ is a target protein binding moiety. The term “target protein binding moiety” refers to a portion of the compound, as set forth herein, that is capable of binding to a target protein. In embodiments, the target protein binding moiety is a monovalent form of a ligand of a target protein.

In embodiments, the target is a Brd4 protein, K-ras protein. Bruton's tyrosine kinase (BTK) protein, androgen receptor (AR) protein, MYC protein, N-MYC protein, beta-catenin protein, or huntingtin (HT) protein.

In embodiments, the target is a Brd4 protein. In embodiments, the target is a K-ras protein. In embodiments, the target is a Bruton's tyrosine kinase (BTK) protein. In embodiments, the target is an androgen receptor (AR) protein. In embodiments, the target is a MYC protein. In embodiments, the target is an N-MYC protein. In embodiments, the target is a beta-catenin protein. In embodiments, the target is a huntingtin (HT) protein.

In embodiments, R³ is

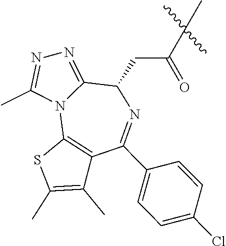 [SMILES: *C(=O)C[C@@H]1N=C(c2ccc(Cl)cc2)c2c(sc(C)c2C)-n2c(C)nnc21]

(i.e., R³ is a Brd4 inhibitor JQ1). Additional Brd4 binding moieties are described in PCT Publication WO2017/197056. The entire contents of this publication is incorporated herein by reference in its entirety for all purposes.

In embodiments, R³ is an azepine derivative such as a derivative having the formula:

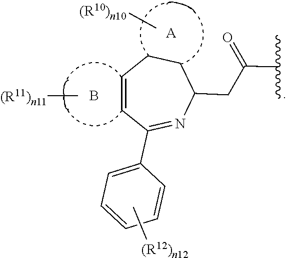

Rings A and B are each independently a C₅-C₆ cycloalkyl, 5 to 6 membered heterocycloalkyl, phenyl, or 5 to 6 membered heteroaryl. For example, rings A and B may include a ring selected from the group consisting of triazo, isoxazolo, thieno, benzo, furanyl, selenophenyl and pyridyl rings. In embodiments, ring A is triazolyl, and ring B is thienyl. In embodiments, ring A is triazolyl, and ring B is benzyl. In embodiments, ring A is isoxazolyl, and ring B is thienyl. In embodiments, ring A is isoxazolyl, and ring B is thienyl.

Each R¹⁰ is independently unsubstituted C₁-C₄ alkyl, —O—R^{10A} or —CF₃, wherein R^{10A} is independently unsubstituted C₁-C₄ alkyl. In embodiments, R¹⁰ is independently unsubstituted methyl. The variable n10 is 0, 1, 2 or 3. In embodiments, n10 is 0. In embodiments, n10 is 1. In embodiments, n10 is 2. In embodiments, n10 is 3.

Each R¹¹ is independently halogen or C₁-C₄ alkyl optionally independently substituted by halogen or hydroxyl. The variable n11 is 0, 1, 2 or 3. In embodiments, n11 is 0. In embodiments, n11 is 1. In embodiments, n11 is 2. In embodiments, n11 is 3.

Each R¹² is independently halogen or phenyl optionally independently substituted by halogen, unsubstituted C₁-C₄ alkyl, unsubstituted C₁-C₄ alkoxy, —CN, —NR¹³—(CH₂)ᵥ₅—R¹⁴ or —NR¹³—C(O)—(CH₂)ᵥ₅—R¹⁴. R¹³ is hydrogen or unsubstituted C₁-C₄ alkyl. The variable v5 is an integer from 0 to 4. R¹⁴ is phenyl optionally substituted by halogen or pyridyl optionally substituted by halogen. The variable n12 is 1 or 2. In embodiments, n12 is 1. In embodiments, n12 is 2.

In embodiments, R³ is a triazolodiazepine derivative such as a derivative having the formula:

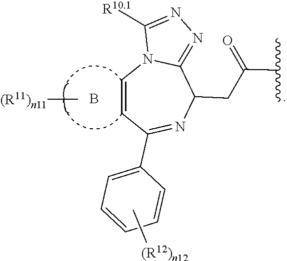

Ring B, R¹¹, n11, R², and n12 are as described herein, including in embodiments. R^{10.1} is hydrogen or any value of R¹⁰ as described herein, including in embodiments. In embodiments, R^{10.1} is unsubstituted methyl.

In embodiments, R³ is a triazolodiazepine derivative such as a derivative having the formula:

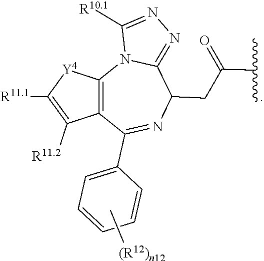

R¹² and n12 are as described herein, including in embodiments. R^{10.1} is hydrogen or any value of R¹⁰ as described herein, including in embodiments. R^{11.1} and R^{11.2} are independently hydrogen or any value of R¹¹ as described herein, including in embodiments. Y⁴ is —S— or —CH═CH—.

In embodiments, Y⁴ is —S—. In embodiments, Y⁴ is —CH═CH—.

In embodiments, R^{10.1} is unsubstituted methyl.

In embodiments, R^{11.1} is hydrogen, halogen, or C₁-C₄ alkyl optionally substituted by halogen or hydroxyl.

In embodiments, R^{11.2} is hydrogen or unsubstituted C₁-C₄ alkyl. In embodiments, R^{11.2} is hydrogen. In embodiments, R^{11.2} is unsubstituted C₁-C₄ alkyl.

In embodiments, R³ is a thienotriazolodiazepine derivative such as (S)-tert-butyl 2-(4-(4-chlorophenyl)-2,3,9-trimethyl-6H-thieno[3,2-f][1,2,4]triazolo[4,3-a][1,4]diazepin-6-yl)acetate (JQ1) and derivatives thereof such as those disclosed in International PCT Publication No. WO2006129623, International PCT Publication No. WO2009084693 and International PCT Publication No. WO2011143651, the contents of which are incorporated herein by reference. Other thienotriazolodiazepine derivatives are disclosed in International PCT Publication No. WO2011143669, the contents of which are incorporated herein by reference.

In embodiments, R³ has the formula:

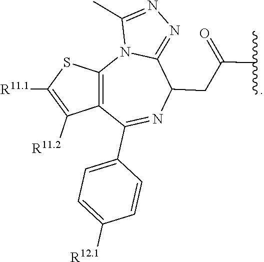

R^{11.1} and R^{11.2} are independently hydrogen or any value of R¹¹ as described herein, including in embodiments. R^{12.1} is hydrogen or any value of R¹² as described herein, including in embodiments.

In embodiments, R^{11.1} is unsubstituted C₁-C₄ alkyl, and R^{11.2} is halogen.

In embodiments, R^{11.1} and R^{11.2} are each unsubstituted methyl.

In embodiments, R^{12.1} is —Cl.

In embodiments, R³ is

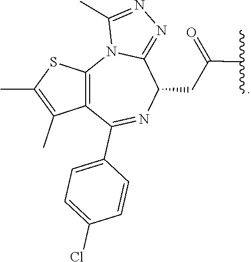 [SMILES: *C(=O)C[C@@H]1N=C(c2ccc(Cl)cc2)c2c(sc(C)c2C)-n2c(C)nnc21]

In embodiments, R³ is

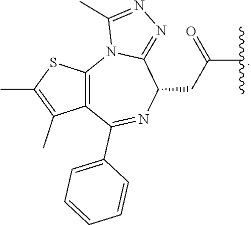 [SMILES: *C(=O)C[C@@H]1N=C(c2ccccc2)c2c(sc(C)c2C)-n2c(C)nnc21]

In embodiments, R³ is a triazolodiazepine derivative such as a derivative having the formula:

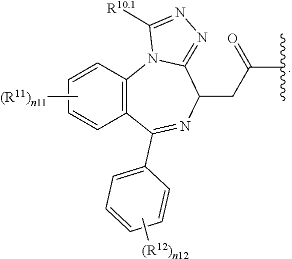

R¹¹, n11, R¹², and n12 are as described herein, including in embodiments. R^{10.1} is hydrogen or any value of R¹⁰ as described herein, including in embodiments. In embodiments, R^{10.1} is unsubstituted methyl.

Triazolobenzodiazepine derivatives include compounds such as benzyl N-(1-methyl-6-phenyl-4H-[1,2,4]triazolo[4,3-a][1,4]benzodiazepin-4-yl)carbamate (GW841819X) and other compounds disclosed in U.S. Pat. No. 5,185,331; 2-[(4S)-6-(4-chlorophenyl)-8-methoxy-1-methyl-4H-[1,2,4]triazolo[4,3-a][1,4]benzodiazepin-4-yl]-N-ethylacetamide (molibresib) and other compounds disclosed in International PCT Publication Nos. WO2011054553, WO2011054844 and WO2011054845, the contents of which are incorporated herein by reference. Other triazolobenzodiazepines may include 8-chloro-1,4-dimethyl-6-phenyl-4h-[1,2,4]triazolo[4,3-A][1,3,4]benzotriazepine such as those compounds disclosed in U.S. Pat. No. 4,163,104 and those disclosed in International PCT Publication No. WO2011161031, the contents of which are incorporated herein by reference.

In embodiments, R³ is:

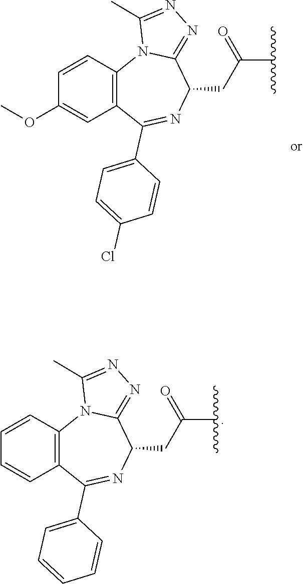 [SMILES: *C(=O)C[C@@H]1N=C(c2ccc(Cl)cc2)c2cc(OC)ccc2-n2c(C)nnc21 ; *C(=O)C[C@@H]1N=C(c2ccccc2)c2ccccc2-n2c(C)nnc21]

In embodiments, R³ is an isoxazoloazepine derivative such as a derivative having the formula:

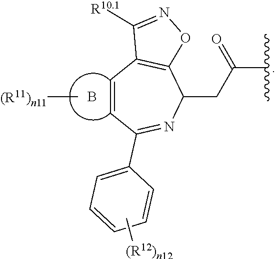

Ring B, R¹¹, n11, R¹², and n12 are as described herein, including in embodiments. R^{10.1} is hydrogen or any value of R¹⁰ as described herein, including in embodiments. In embodiments, R^{10.1} is unsubstituted methyl.

In embodiments, R³ is a isoxazoloazepine derivative such as a derivative having the formula:

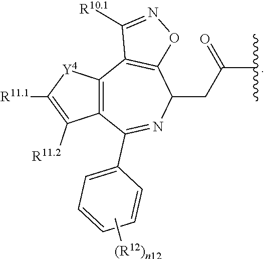

R¹² and n12 are as described herein, including in embodiments. R^{10.1} is hydrogen or any value of R¹⁰ as described herein, including in embodiments. R_{10.1} and R^{11.2} are independently hydrogen or any value of R¹ as described herein, including in embodiments. Y⁴ is —S— or —CH═CH—.

In embodiments, Y⁴ is —S—. In embodiments, Y⁴ is —CH═CH—.

In embodiments, R³ has the formula:

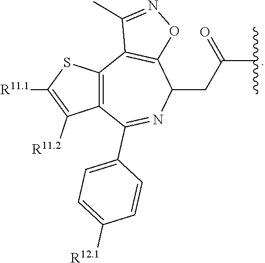

R^{11.1} and R^{11.2} are independently hydrogen or any value of R¹¹ as described herein, including in embodiments. R^{12.1} is hydrogen or any value of R¹² as described herein, including in embodiments.

In embodiments, R³ is a thienoisoxazoloazepine derivative such as (S)-2-(4-(4-chlorophenyl)-2,3,9-trimethyl-6H-isoxazolo[5,4-c]thieno[2,3-e]azepin-6-yl)acetamide (CPI-3) and derivatives thereof such as those disclosed in Gehling et al., Discovery, Design, and Optimization of Isoxazole Azepine BET Inhibitors, ACS Med. Chem. Lett. 2013, 4, 835-840 and M. C. Hewitt et al., Development of methyl isoxazoloazepines as inhibitors of BET, Bioorg. Med. Chem. Lett. 25 (2015) 1842-1848, the contents of which are incorporated herein by reference.

In embodiments, R³ is

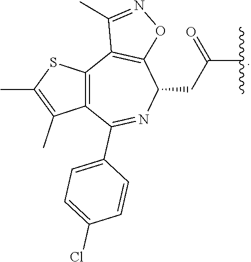 [SMILES: *C(=O)C[C@@H]1N=C(c2ccc(Cl)cc2)c2c(sc(C)c2C)-c2c(C)noc21]

In embodiments, R³ is a benzoisoxazoloazepine derivative such as a derivative having the formula:

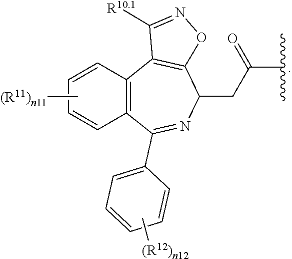

R¹¹, n11, R¹², and n12 are as described herein, including in embodiments. R^{10.1} is hydrogen or any value of R¹⁰ as described herein, including in embodiments. In embodiments, R^{10.1} is unsubstituted methyl.

Benzoisoxazoloazepine derivatives include compounds such as 2-[(4S)-6-(4-chlorophenyl)-1-methyl-4H-[1,2]oxazolo[5,4-d][2]benzazepin-4-yl]acetamide (CPI-0610) as described in Albrecht et al., Identification of a Benzoisoxazoloazepine Inhibitor (CPI-0610) of the Bromodomain and Extra-Terminal (BET) Family as a Candidate for Human Clinical Trials, J. Med. Chem. 2016, 59, 1330-1339 and International PCT Publication No. WO2012075383, the contents of which are incorporated herein by reference.

In embodiments, R³ is:

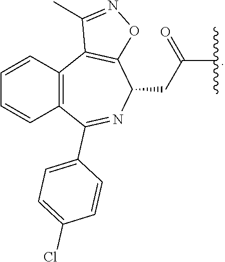 [SMILES: *C(=O)C[C@@H]1N=C(c2ccc(Cl)cc2)c2ccccc2-c2c(C)noc21]

In embodiments, R³ is a monovalent form of GSK046 disclosed in Gilan et al., Science 368, 387-394 (2020) having the formula:

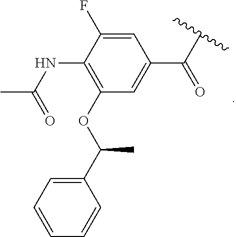 [SMILES: *C(=O)c1cc(F)c(NC(C)=O)c(O[C@@H](C)c2ccccc2)c1]

In embodiments, R³ is a moiety (e.g., monovalent form) of a compound selected from compounds disclosed in International PCT Publication No. WO2017/037116, the contents of which are incorporated herein by reference, such as GSK-620 having the formula:

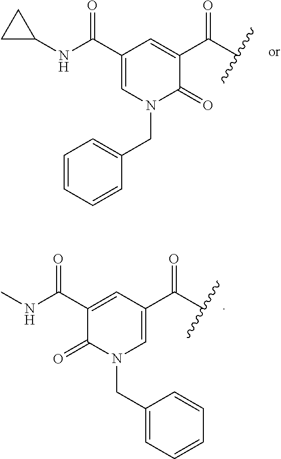 [SMILES: *C(=O)c1cc(C(=O)NC2CC2)cn(Cc2ccccc2)c1=O ; *C(=O)c1cc(C(=O)NC)c(=O)n(Cc2ccccc2)c1]

In embodiments, R³ is:

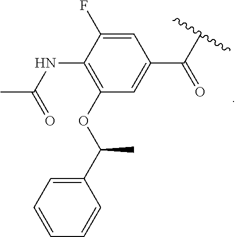 [SMILES: *C(=O)c1cc(F)c(NC(C)=O)c(O[C@@H](C)c2ccccc2)c1]

In embodiments, R³ is:

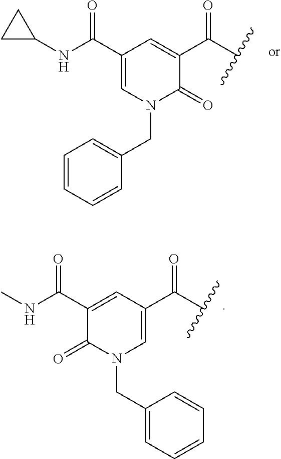 [SMILES: *C(=O)c1cc(C(=O)NC2CC2)cn(Cc2ccccc2)c1=O ; *C(=O)c1cc(C(=O)NC)c(=O)n(Cc2ccccc2)c1]

In embodiments, the Brd4 binding moiety binds to Brd4 with a Kd of less than one micromolar. In embodiments, the Brd4 binding moiety binds to Brd4 with a Kd of less than 500 nanomolar. In embodiments, the Brd4 binding moiety binds to Brd4 with a Kd of less than 450 nanomolar. In embodiments, the Brd4 binding moiety binds to Brd4 with a Kd of les than 400 nanomolar. In embodiments, the Brd4 binding moiety binds to Brd4 with a Kd of less than 350 nanomolar. In embodiments, the Brd4 binding moiety binds to Brd4 with a Kd of less than 300 nanomolar. In embodiments, the Brd4 binding moiety binds to Brd4 with a Kd of less than 250 nanomolar. In embodiments, the Brd4 binding moiety binds to Brd4 with a Kd of less than 200 nanomolar. In embodiments, the Brd4 binding moiety binds to Brd4 with a Kd of less than 180 nanomolar. In embodiments, the Brd4 binding moiety binds to Brd4 with a Kd of less than 150 nanomolar. In embodiments, the Brd4 binding moiety binds to Brd4 with a Kd of less than 100 nanomolar. In embodiments, the Brd4 binding moiety binds to Brd4 with a Kd of less than 50 nanomolar. In embodiments, the Brd4 binding moiety binds to Brd4 with a Kd of less than 25 nanomolar. In embodiments, the Brd4 binding moiety binds to Brd4 with a Kd of less than 15 nanomolar. In embodiments, the Brd4 binding moiety binds to Brd4 with a Kd of less than 10 nanomolar. In embodiments, the Brd4 binding moiety binds to Brd4 with a Kd of less than 5 nanomolar. In embodiments, the Brd4 binding moiety binds to Brd4 with a Kd of less than one nanomolar. In embodiments, the Brd4 binding moiety binds to Brd4 with a Kd between 180 nM and 15 nM.

In embodiments, the K-ras binding moiety binds to K-ras with a Kd of less than one micromolar. In embodiments, the K-ras binding moiety binds to K-ras with a Kd of less than 500 nanomolar. In embodiments, the K-ras binding moiety binds to K-ras with a Kd of less than 450 nanomolar. In embodiments, the K-ras binding moiety binds to K-ras with a Kd of less than 400 nanomolar. In embodiments, the K-ras binding moiety binds to K-ras with a Kd of less than 350 nanomolar. In embodiments, the K-ras binding moiety binds to K-ras with a Kd of less than 300 nanomolar. In embodiments, the K-ras binding moiety binds to K-ras with a Kd of less than 250 nanomolar. In embodiments, the K-ras binding moiety binds to K-ras with a Kd of less than 200 nanomolar. In embodiments, the K-ras binding moiety binds to K-ras with a Kd of less than 180 nanomolar. In embodiments, the K-ras binding moiety binds to K-ras with a Kd of less than 150 nanomolar. In embodiments, the K-ras binding moiety binds to K-ras with a Kd of less than 100 nanomolar. In embodiments, the K-ras binding moiety binds to K-ras with a Kd of less than 50 nanomolar. In embodiments, the K-ras binding moiety binds to K-ras with a Kd of less than 25 nanomolar. In embodiments, the K-ras binding moiety binds to K-ras with a Kd of less than 15 nanomolar. In embodiments, the K-ras binding moiety binds to K-ras with a Kd of less than 10 nanomolar. In embodiments, the K-ras binding moiety binds to K-ras with a Kd of less than 5 nanomolar. In embodiments, the K-ras binding moiety binds to K-ras with a Kd of less than one nanomolar. In embodiments, the K-ras binding moiety binds to K-ras with a Kd between 180 nM and 15 nM.

In embodiments, the BTK binding moiety binds to BTK with a Kd of less than one micromolar. In embodiments, the BTK binding moiety binds to BTK with a Kd of less than 500 nanomolar. In embodiments, the BTK binding moiety binds to BTK with a Kd of less than 450 nanomolar. In embodiments, the BTK binding moiety binds to BTK with a Kd of less than 400 nanomolar. In embodiments, the BTK binding moiety binds to BTK with a Kd of less than 350 nanomolar. In embodiments, the BTK binding moiety binds to BTK with a Kd of less than 300 nanomolar. In embodiments, the BTK binding moiety binds to BTK with a Kd of less than 250 nanomolar. In embodiments, the BTK binding moiety binds to BTK with a Kd of less than 200 nanomolar. In embodiments, the BTK binding moiety binds to BTK with a Kd of less than 180 nanomolar. In embodiments, the BTK binding moiety binds to BTK with a Kd of less than 150 nanomolar. In embodiments, the BTK binding moiety binds to BTK with a Kd of less than 100 nanomolar. In embodiments, the BTK binding moiety binds to BTK with a Kd of less than 50 nanomolar. In embodiments, the BTK binding moiety binds to BTK with a Kd of less than 25 nanomolar. In embodiments, the BTK binding moiety binds to BTK with a Kd of less than 15 nanomolar. In embodiments, the BTK binding moiety binds to BTK with a Kd of less than 10 nanomolar. In embodiments, the BTK binding moiety binds to BTK with a Kd of less than 5 nanomolar. In embodiments, the BTK binding moiety binds to BTK with a Kd of less than one nanomolar. In embodiments, the BTK binding moiety binds to BTK with a Kd between 180 nM and 15 nM.

In embodiments, the AR binding moiety binds to AR with a Kd of less than one micromolar. In embodiments, the AR binding moiety binds to AR with a Kd of less than 500 nanomolar. In embodiments, the AR binding moiety binds to AR with a Kd of less than 450 nanomolar. In embodiments, the AR binding moiety binds to AR with a Kd of less than 400 nanomolar. In embodiments, the AR binding moiety binds to AR with a Kd of less than 350 nanomolar. In embodiments, the AR binding moiety binds to AR with a Kd of less than 300 nanomolar. In embodiments, the AR binding moiety binds to AR with a Kd of less than 250 nanomolar. In embodiments, the AR binding moiety binds to AR with a Kd of less than 200 nanomolar. In embodiments, the AR binding moiety binds to AR with a Kd of less than 180 nanomolar. In embodiments, the AR binding moiety binds to AR with a Kd of less than 150 nanomolar. In embodiments, the AR binding moiety binds to AR with a Kd of less than 100 nanomolar. In embodiments, the AR binding moiety binds to AR with a Kd of less than 50 nanomolar. In embodiments, the AR binding moiety binds to AR with a Kd of less than 25 nanomolar. In embodiments, the AR binding moiety binds to AR with a Kd of less than 15 nanomolar. In embodiments, the AR binding moiety binds to AR with a Kd of less than 10 nanomolar. In embodiments, the AR binding moiety binds to AR with a Kd of less than 5 nanomolar. In embodiments, the AR binding moiety binds to AR with a Kd of less than one nanomolar. In embodiments, the AR binding moiety binds to AR with a Kd between 180 nM and 15 nM.

In embodiments, the MYC binding moiety binds to MYC with a Kd of less than one micromolar. In embodiments, the MYC binding moiety binds to MYC with a Kd of less than 500 nanomolar. In embodiments, the MYC binding moiety binds to MYC with a Kd of less than 450 nanomolar. In embodiments, the MYC binding moiety binds to MYC with a Kd of less than 400 nanomolar. In embodiments, the MYC binding moiety binds to MYC with a Kd of less than 350 nanomolar. In embodiments, the MYC binding moiety binds to MYC with a Kd of less than 300 nanomolar. In embodiments, the MYC binding moiety binds to MYC with a Kd of less than 250 nanomolar. In embodiments, the MYC binding moiety binds to MYC with a Kd of less than 200 nanomolar. In embodiments, the MYC binding moiety binds to MYC with a Kd of less than 180 nanomolar. In embodiments, the MYC binding moiety binds to MYC with a Kd of less than 150 nanomolar. In embodiments, the MYC binding moiety binds to MYC with a Kd of less than 100 nanomolar. In embodiments, the MYC binding moiety binds to MYC with a Kd of less than 50 nanomolar. In embodiments, the MYC binding moiety binds to MYC with a Kd of less than 25 nanomolar. In embodiments, the MYC binding moiety binds to MYC with a Kd of less than 15 nanomolar. In embodiments, the MYC binding moiety binds to MYC with a Kd of less than 10 nanomolar. In embodiments, the MYC binding moiety binds to MYC with a Kd of less than 5 nanomolar. In embodiments, the MYC binding moiety binds to MYC with a Kd of less than one nanomolar. In embodiments, the MYC binding moiety binds to MYC with a Kd between 180 nM and 15 nM.

In embodiments, the N-MYC binding moiety binds to N-MYC with a Kd of less than one micromolar. In embodiments, the N-MYC binding moiety binds to N-MYC with a Kd of less than 500 nanomolar. In embodiments, the N-MYC binding moiety binds to N-MYC with a Kd of less than 450 nanomolar. In embodiments, the N-MYC binding moiety binds to N-MYC with a Kd of less than 400 nanomolar. In embodiments, the N-MYC binding moiety binds to N-MYC with a Kd of less than 350 nanomolar. In embodiments, the N-MYC binding moiety binds to N-MYC with a Kd of less than 300 nanomolar. In embodiments, the N-MYC binding moiety binds to N-MYC with a Kd of less than 250 nanomolar. In embodiments, the N-MYC binding moiety binds to N-MYC with a Kd of less than 200 nanomolar. In embodiments, the N-MYC binding moiety binds to N-MYC with a Kd of less than 180 nanomolar. In embodiments, the N-MYC binding moiety binds to N-MYC with a Kd of less than 150 nanomolar. In embodiments, the N-MYC binding moiety binds to N-MYC with a Kd of less than 100 nanomolar. In embodiments, the N-MYC binding moiety binds to N-MYC with a Kd of less than 50 nanomolar. In embodiments, the N-MYC binding moiety binds to N-MYC with a Kd of less than 25 nanomolar. In embodiments, the N-MYC binding moiety binds to N-MYC with a Kd of less than 15 nanomolar. In embodiments, the N-MYC binding moiety binds to N-MYC with a Kd of less than 10 nanomolar. In embodiments, the N-MYC binding moiety binds to N-MYC with a Kd of less than 5 nanomolar. In embodiments, the N-MYC binding moiety binds to N-MYC with a Kd of less than one nanomolar. In embodiments, the N-MYC binding moiety binds to N-MYC with a Kd between 180 nM and 15 nM.

In embodiments, the beta-catenin binding moiety binds to beta-catenin with a Kd of less than one micromolar. In embodiments, the beta-catenin binding moiety binds to beta-catenin with a Kd of less than 500 nanomolar. In embodiments, the beta-catenin binding moiety binds to beta-catenin with a Kd of less than 450 nanomolar. In embodiments, the beta-catenin binding moiety binds to beta-catenin with a Kd of less than 400 nanomolar. In embodiments, the beta-catenin binding moiety binds to beta-catenin with a Kd of less than 350 nanomolar. In embodiments, the beta-catenin binding moiety binds to beta-catenin with a Kd of less than 300 nanomolar. In embodiments, the beta-catenin binding moiety binds to beta-catenin with a Kd of less than 250 nanomolar. In embodiments, the beta-catenin binding moiety binds to beta-catenin with a Kd of less than 200 nanomolar. In embodiments, the beta-catenin binding moiety binds to beta-catenin with a Kd of less than 180 nanomolar. In embodiments, the beta-catenin binding moiety binds to beta-catenin with a Kd of less than 150 nanomolar. In embodiments, the beta-catenin binding moiety binds to beta-catenin with a Kd of less than 100 nanomolar. In embodiments, the beta-catenin binding moiety binds to beta-catenin with a Kd of less than 50 nanomolar. In embodiments, the beta-catenin binding moiety binds to beta-catenin with a Kd of less than 25 nanomolar. In embodiments, the beta-catenin binding moiety binds to beta-catenin with a Kd of less than 15 nanomolar. In embodiments, the beta-catenin binding moiety binds to beta-catenin with a Kd of less than 10 nanomolar. In embodiments, the beta-catenin binding moiety binds to beta-catenin with a Kd of less than 5 nanomolar. In embodiments, the beta-catenin binding moiety binds to beta-catenin with a Kd of less than one nanomolar. In embodiments, the beta-catenin binding moiety binds to beta-catenin with a Kd between 180 nM and 15 nM.

In embodiments, the HTT binding moiety binds to HTT with a Kd of less than one micromolar. In embodiments, the HTT binding moiety binds to HTT with a Kd of less than 500 nanomolar. In embodiments, the HTT binding moiety binds to HTT with a Kd of less than 450 nanomolar. In embodiments, the HTT binding moiety binds to HTT with a Kd of less than 400 nanomolar. In embodiments, the HTT binding moiety binds to HTT with a Kd of less than 350 nanomolar. In embodiments, the HTT binding moiety binds to HTT with a Kd of less than 300 nanomolar. In embodiments, the HTT binding moiety binds to HTT with a Kd of less than 250 nanomolar. In embodiments, the HTT binding moiety binds to HTT with a Kd of less than 200 nanomolar. In embodiments, the HTT binding moiety binds to HTT with a Kd of less than 180 nanomolar. In embodiments, the HTT binding moiety binds to HTT with a Kd of less than 150 nanomolar. In embodiments, the HTT binding moiety binds to HTT with a Kd of less than 100 nanomolar. In embodiments, the HTT binding moiety binds to HTT with a Kd of less than 50 nanomolar. In embodiments, the HTT binding moiety binds to HTT with a Kd of less than 25 nanomolar. In embodiments, the HTT binding moiety binds to HTT with a Kd of less than 15 nanomolar. In embodiments, the HTT binding moiety binds to HTT with a Kd of less than 10 nanomolar. In embodiments, the HTT binding moiety binds to HTT with a Kd of less than 5 nanomolar. In embodiments, the HTT binding moiety binds to HTT with a Kd of less than one nanomolar. In embodiments, the HTT binding moiety binds to HTT with a Kd between 180 nM and 15 nM.

In embodiments, z1 is 1 or 2. In embodiments, z1 is 1. In embodiments, z1 is 2.

In embodiments, z2 is 0 to 5. In embodiments, z2 is 0. In embodiments, z2 is 1. In embodiments, z2 is 2. In embodiments, z2 is 3. In embodiments, z2 is 4. In embodiments, z2 is 5.

In embodiments, z3 is 0 to 3. In embodiments, z3 is 0. In embodiments, z3 is 1. In embodiments, z3 is 2. In embodiments, z3 is 3.

In embodiments, z4 is 0 or 1. In embodiments, z4 is 0. In embodiments, z4 is 1.

In embodiments, z5 is 0 to 4. In embodiments, z5 is 0. In embodiments, z5 is 1. In embodiments, z5 is 2. In embodiments, z5 is 3. In embodiments, z5 is 4.

In embodiments, z6 is 0 to 4. In embodiments, z6 is 0. In embodiments, z6 is 1. In embodiments, z6 is 2. In embodiments, z6 is 3. In embodiments, z6 is 4.

In embodiments, z7 is 0 to 2. In embodiments, z7 is 0. In embodiments, z7 is 1. In embodiments, z7 is 2.

In embodiments, z8 is 0 to 3. In embodiments, z8 is 0. In embodiments, z8 is 1. In embodiments, z8 is 2. In embodiments, z8 is 3.

In embodiments, z9 is 0 to 4. In embodiments, z9 is 0. In embodiments, z9 is 1. In embodiments, z9 is 2. In embodiments, z9 is 3. In embodiments, z9 is 4.

In embodiments, z10 is 0 to 3. In embodiments, z10 is 0. In embodiments, z10 is 1.

In embodiments, z10 is 2. In embodiments, z10 is 3.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when two adjacent R² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R¹⁰¹ is substituted, R¹⁰¹ is substituted with one or more first substituent groups denoted by R^{101.1} as explained in the definitions section above in the description of “first substituent group(s)”. In embodiments, when an R^{101.1} substituent group is substituted, the R^{101.1} substituent group is substituted with one or more second substituent groups denoted by R^{101.2} as explained in the definitions section above in the description of “first substituent group(s)”. In embodiments, when an R^{101.2} substituent group is substituted, the R^{101.2} substituent group is substituted with one or more third substituent groups denoted by R^{101.3} as explained in the definitions section above in the description of “first substituent group(s)”. In the above embodiments, R¹⁰¹, R^{101.1}, R^{101.2}, and R^{101.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰¹, R^{101.1}, R^{101.2}, and R^{101.3}, respectively.

In embodiments, when R¹⁰² is substituted, R¹⁰² is substituted with one or more first substituent groups denoted by R^{102.1} as explained in the definitions section above in the description of “first substituent group(s)”. In embodiments, when an R^{102.1} substituent group is substituted, the R^{102.1} substituent group is substituted with one or more second substituent groups denoted by R^{102.2} as explained in the definitions section above in the description of “first substituent group(s)”. In embodiments, when an R^{102.2} substituent group is substituted, the R^{102.2} substituent group is substituted with one or more third substituent groups denoted by R^{102.3} as explained in the definitions section above in the description of “first substituent group(s)”. In the above embodiments, R¹⁰², R^{102.1}, R^{102.2}, and R^{102.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰², R^{102.1}, R^{102.2}, and R^{102.3}, respectively.

In embodiments, when R¹⁰³ is substituted, R¹⁰³ is substituted with one or more first substituent groups denoted by R^{103.1} as explained in the definitions section above in the description of “first substituent group(s)”. In embodiments, when an R^{103.1} substituent group is substituted, the R^{103.1} substituent group is substituted with one or more second substituent groups denoted by R^{103.2} as explained in the definitions section above in the description of “first substituent group(s)”. In embodiments, when an R^{103.2} substituent group is substituted, the R^{103.2} substituent group is substituted with one or more third substituent groups denoted by R^{103.3} as explained in the definitions section above in the description of “first substituent group(s)”. In the above embodiments, R¹⁰³, R^{103.1}, R^{103.2}, and R^{103.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰³, R^{103.1}, R^{103.2}, and R^{103.3}, respectively.

In embodiments, when R^{101A} is substituted, R^{101A} is substituted with one or more first substituent groups denoted by R^{101A.1} as explained in the definitions section above in the description of “first substituent group(s)”. In embodiments, when an R^{101A.1} substituent group is substituted, the R^{101A.1} substituent group is substituted with one or more second substituent groups denoted by R^{101A.2} as explained in the definitions section above in the description of “first substituent group(s)”. In embodiments, when an R^{101A.2} substituent group is substituted, the R^{101A.2} substituent group is substituted with one or more third substituent groups denoted by R^{101A.3} as explained in the definitions section above in the description of “first substituent group(s)”. In the above embodiments, R^{101A}, R^{101A.1}, R^{101A.2}, and R^{101A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{101A}, R^{101A.1}, R^{101A.2}, and R^{101A.3}, respectively.

In embodiments, when two adjacent R^{101A} substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{101A.1} as explained in the definitions section above in the description of “first substituent group(s)”. In embodiments, when an R^{101A.1} substituent group is substituted, the R^{101A.1} substituent group is substituted with one or more second substituent groups denoted by R^{101A.2} as explained in the definitions section above in the description of “first substituent group(s)”. In embodiments, when an R^{101A.2} substituent group is substituted, the R^{101A.2} substituent group is substituted with one or more third substituent groups denoted by R^{101A.3} as explained in the definitions section above in the description of “first substituent group(s)”. In the above embodiments, R^{101A}, R^{101A.1}, R^{101A.2}, and R^{101A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{101A}, R^{101A.1}, R^{101A.2}, and R^{101A.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², R^{L2.1}, R^{L2.2}, and R^{L2.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L3.3} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³, R^{L3.1}, R^{L3.2}, and R^{L3.3}, respectively.

In embodiments, when L^{3A} is substituted, L^{3A} is substituted with one or more first substituent groups denoted by R^{L3A.1} as explained in the definitions section above in the description of “first substituent group(s)”. In embodiments, when an R^{L3A.1} substituent group is substituted, the R^{L3A.1} substituent group is substituted with one or more second substituent groups denoted by R^{L3A.2} explained in the definitions section above in the description of “first substituent group(s)”. In embodiments, when an R^{L3A.2} substituent group is substituted, the R^{L3A.2} substituent group is substituted with one or more third substituent groups denoted by R^{L3A.3} as explained in the definitions section above in the description of “first substituent group(s)”. In the above embodiments, L^{3A}, R^{L3A.1}, R^{L3A.2}, and R^{L3A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3A}, R^{L3A.1}, R^{L3A.2}, and R^{L3A.3}, respectively.

In embodiments, when L^{3B} is substituted, L^{3B} is substituted with one or more first substituent groups denoted by R^{L3B.1} as explained in the definitions section above in the description of “first substituent group(s)”. In embodiments, when an R^{L3B.1} substituent group is substituted, the R^{L3B.1} substituent group is substituted with one or more second substituent groups denoted by R^{L3B.2} as explained in the definitions section above in the description of “first substituent group(s)”. In embodiments, when an R^{L3B.2} substituent group is substituted, the R^{L3B.2} substituent group is substituted with one or more third substituent groups denoted by R^{L3B.3} as explained in the definitions section above in the description of “first substituent group(s)”. In the above embodiments, L^{3B}, R^{L3B.1}, R^{L3B.2}, and R^{L3B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³B, R^{L3B.1}, R^{L3B.2}, and R^{L3B.3}, respectively.

In embodiments, when L^{3C} is substituted, L^{3C} is substituted with one or more first substituent groups denoted by R^{L3C.1} as explained in the definitions section above in the description of “first substituent group(s)”. In embodiments, when an R^{L3C.1} substituent group is substituted, the R^{L3C.1} substituent group is substituted with one or more second substituent groups denoted by R^{L3C.2} as explained in the definitions section above in the description of “first substituent group(s)”. In embodiments, when an R^{L3C.2} substituent group is substituted, the R^{L3C.2} substituent group is substituted with one or more third substituent groups denoted by R^{L3C.3} as explained in the definitions section above in the description of “first substituent group(s)”. In the above embodiments, L^{3C}, R^{L3C.1}, R^{L3C.2}, and R^{L3C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3C}, R^{L3C.1}, R^{L3C.2}, and R^{L3C.3}, respectively.

In embodiments, when R¹⁵ is substituted, R¹⁵ is substituted with one or more first substituent groups denoted by R^{15.1} as explained in the definitions section above in the description of “first substituent group(s)”. In embodiments, when an R^{15.1} substituent group is substituted, the R^{15.1} substituent group is substituted with one or more second substituent groups denoted by R^{15.2} as explained in the definitions section above in the description of “first substituent group(s)”. In embodiments, when an R^{15.2} substituent group is substituted, the R^{15.2} substituent group is substituted with one or more third substituent groups denoted by R^{15.3} as explained in the definitions section above in the description of “first substituent group(s)”. In the above embodiments, R¹⁵, R^{15.1}, R^{15.2}, and R^{15.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁵, R^{15.1}, R^{15.2}, and R^{15.3}, respectively.

In embodiments, when R¹⁶ is substituted, R¹⁶ is substituted with one or more first substituent groups denoted by R^{16.1} as explained in the definitions section above in the description of “first substituent group(s)”. In embodiments, when an R^{16.1} substituent group is substituted, the R^{16.1} substituent group is substituted with one or more second substituent groups denoted by R^{16.2} as explained in the definitions section above in the description of “first substituent group(s)”. In embodiments, when an R^{16.2} substituent group is substituted, the R^{16.2} substituent group is substituted with one or more third substituent groups denoted by R^{16.3} as explained in the definitions section above in the description of “first substituent group(s)”. In the above embodiments, R¹⁶, R^{16.1}, R^{16.2}, and R^{16.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁶, R^{16.1}, R^{16.2}, and R^{16.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R¹⁸ is substituted, R¹⁸ is substituted with one or more first substituent groups denoted by R^{18.1} as explained in the definitions section above in the description of “first substituent group(s)”. In embodiments, when an R¹⁵ substituent group is substituted, the R^{18.1} substituent group is substituted with one or more second substituent groups denoted by R^{18.2} as explained in the definitions section above in the description of “first substituent group(s)”. In embodiments, when an R^{18.2} substituent group is substituted, the R^{18.2} substituent group is substituted with one or more third substituent groups denoted by R^{18.3} as explained in the definitions section above in the description of “first substituent group(s)”. In the above embodiments, R¹⁸, R^{18.1}, R^{18.2}, and R^{18.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁸, R^{18.1}, R^{18.2}, and R^{18.3}, respectively.

In embodiments, when R¹⁹ is substituted, R¹⁹ is substituted with one or more first substituent groups denoted by R^{19.1} as explained in the definitions section above in the description of “first substituent group(s)”. In embodiments, when an R^{19.1} substituent group is substituted, the R^{19.1} substituent group is substituted with one or more second substituent groups denoted by R^{19.2} as explained in the definitions section above in the description of “first substituent group(s)”. In embodiments, when an R^{19.2} substituent group is substituted, the R^{19.2} substituent group is substituted with one or more third substituent groups denoted by R^{19.3} as explained in the definitions section above in the description of “first substituent group(s)”. In the above embodiments, R¹⁹, R^{19.1}, R^{19.2}, and R^{19.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁹, R^{19.1}, R^{19.2}, and R^{19.3}, respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰, R^{20.1}, R^{20.2}, and R^{20.3}, respectively.

In an aspect, provided herein is a compound selected from a group consisting of:

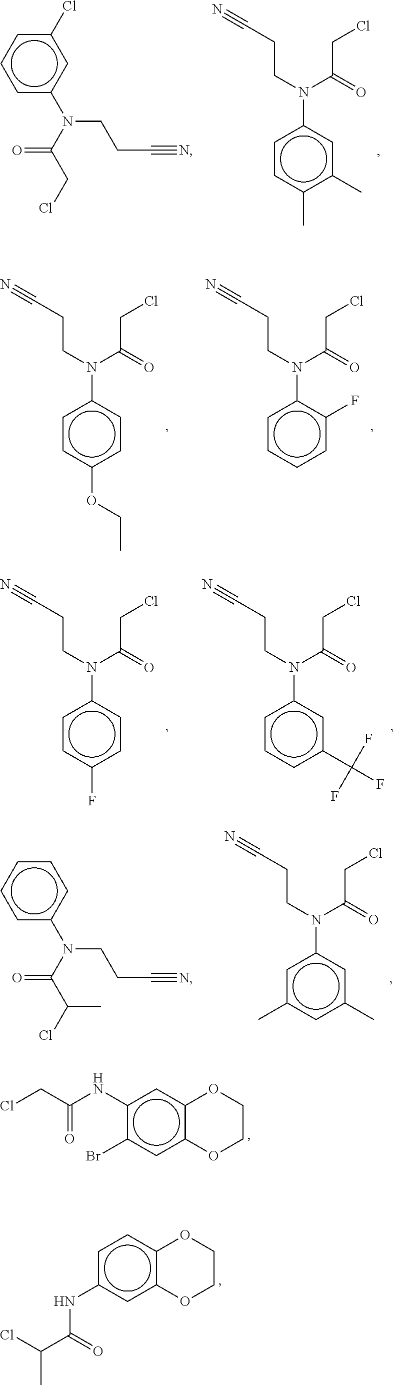

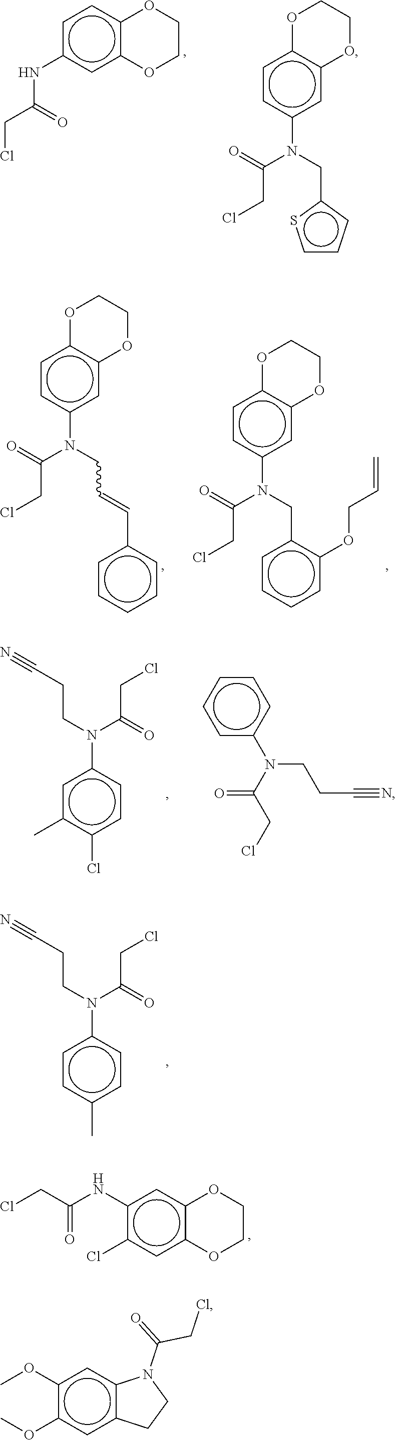

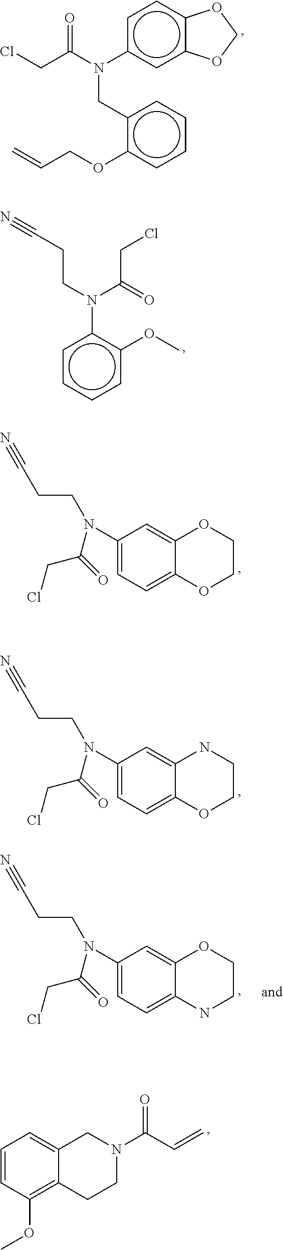

or a pharmaceutically acceptable salt thereof.

In embodiments, the compound is

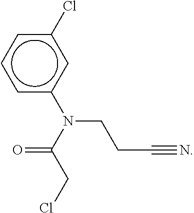

In embodiments, the compound is

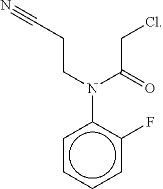

In embodiments, the compound is

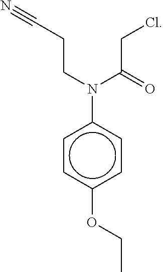

In embodiments, the compound is

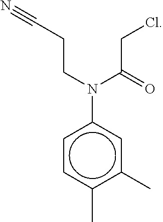

In embodiments, the compound is

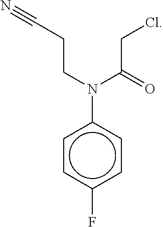

In embodiments, the compound is

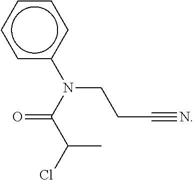

In embodiments, the compound is

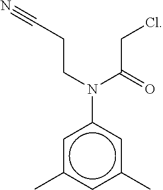

In embodiments, the compound is

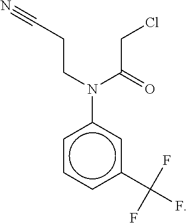

In embodiments, the compound is

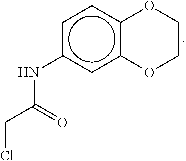

In embodiments, the compound is

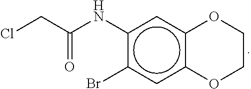

In embodiments, the compound is

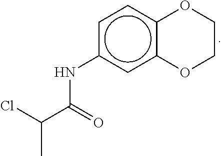

In embodiments, the compound is

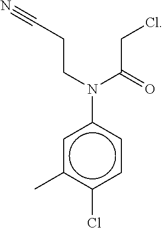

In embodiments, the compound is

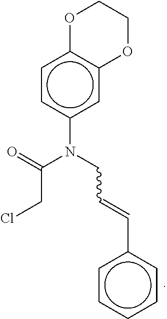

In embodiments, the compound is

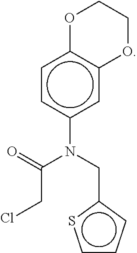

In embodiments, the compound is

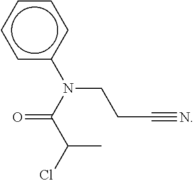

In embodiments, the compound is

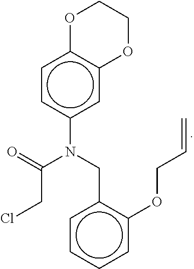

In embodiments, the compound is

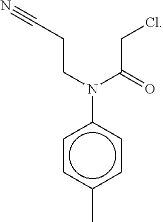

In embodiments, the compound is

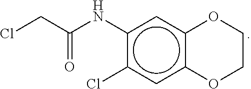

In embodiments, the compound is

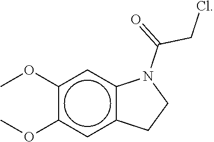

In embodiments, the compound is

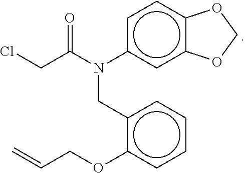

In embodiments, the compound is

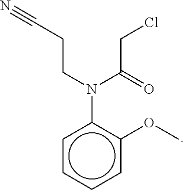

In embodiments, the compound is

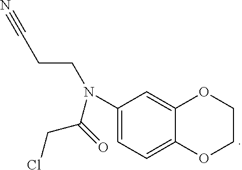 [SMILES: N#CCCN(C(=O)CCl)c1ccc2c(c1)OCCO2]

In embodiments, the compound is

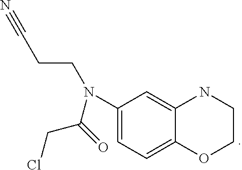 [SMILES: N#CCCN(C(=O)CCl)c1ccc2c(c1)NCCO2]

In embodiments, the compound is

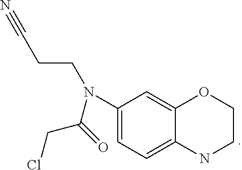 [SMILES: N#CCCN(C(=O)CCl)c1ccc2c(c1)OCCN2]

In embodiments, the compound is

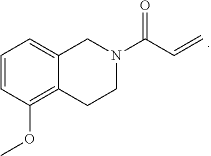 [SMILES: C=CC(=O)N1CCc2c(cccc2OC)C1]

In embodiments, the compound is

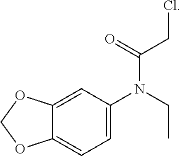 [SMILES: CCN(C(=O)CCl)c1ccc2c(c1)OCO2]

In embodiments, the compound is

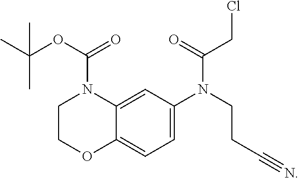 [SMILES: CC(C)(C)OC(=O)N1CCOc2ccc(N(CCC#N)C(=O)CCl)cc21]

In embodiments, the compound is

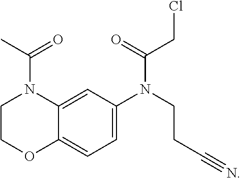 [SMILES: CC(=O)N1CCOc2ccc(N(CCC#N)C(=O)CCl)cc21]

In embodiments, the compound is

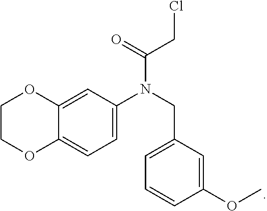 [SMILES: COc1cccc(CN(C(=O)CCl)c2ccc3c(c2)OCCO3)c1]

In embodiments, the compound is

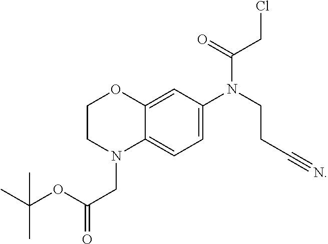 [SMILES: CC(C)(C)OC(=O)CN1CCOc2cc(N(CCC#N)C(=O)CCl)ccc21]

In embodiments, the compound is

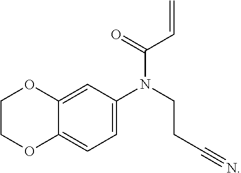 [SMILES: C=CC(=O)N(CCC#N)c1ccc2c(c1)OCCO2]

In embodiments, the compound is

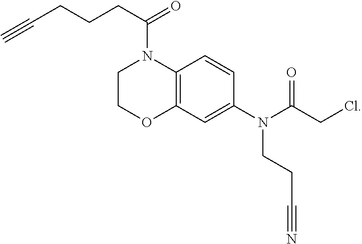 [SMILES: C#CCCCC(=O)N1CCOc2cc(N(CCC#N)C(=O)CCl)ccc21]

In embodiments, the compound is

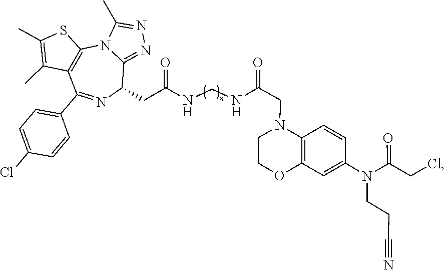

wherein n is an integer from 1 to 8.

In embodiments, the compound is

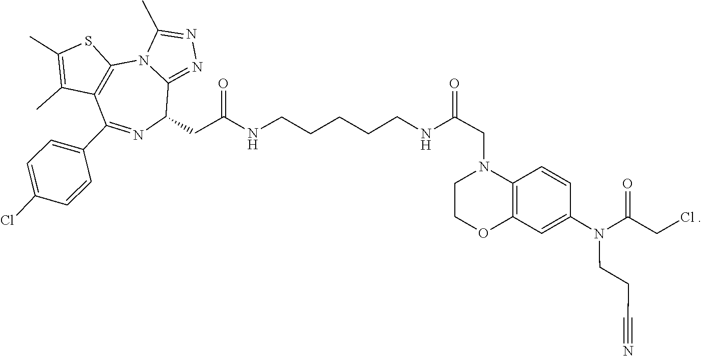 [SMILES: Cc1sc2c(c1C)C(c1ccc(Cl)cc1)=N[C@@H](CC(=O)NCCCCCNC(=O)CN1CCOc3cc(N(CCC#N)C(=O)CCl)ccc31)c1nnc(C)n1-2]

In embodiments, the compound is

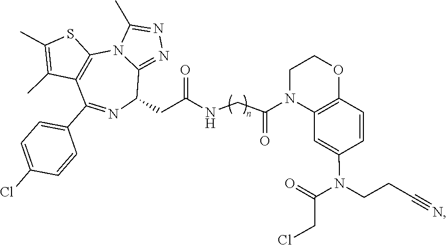

wherein n is an integer from 1 to 8.

In embodiments, the compound is

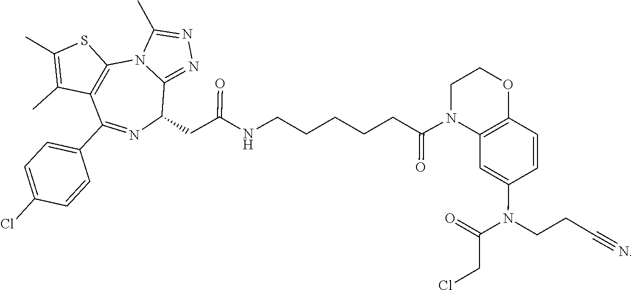 [SMILES: Cc1sc2c(c1C)C(c1ccc(Cl)cc1)=N[C@@H](CC(=O)NCCCCCC(=O)N1CCOc3ccc(N(CCC#N)C(=O)CCl)cc31)c1nnc(C)n1-2]

In embodiments, the compound is

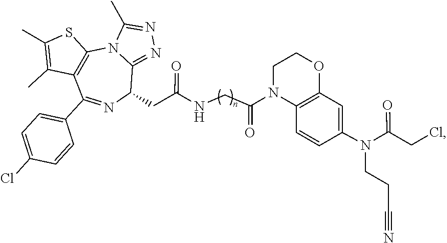

wherein n is an integer from 1 to 8.

In embodiments, the compound is

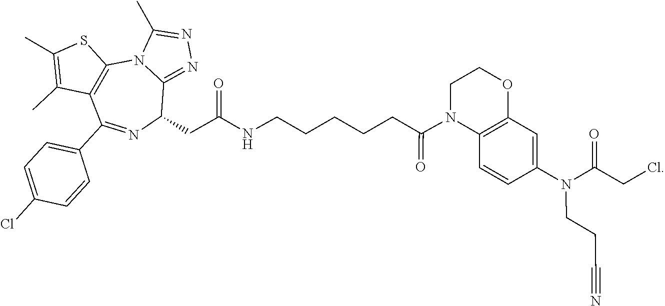 [SMILES: Cc1sc2c(c1C)C(c1ccc(Cl)cc1)=N[C@@H](CC(=O)NCCCCCC(=O)N1CCOc3cc(N(CCC#N)C(=O)CCl)ccc31)c1nnc(C)n1-2]

In embodiments, the compound is

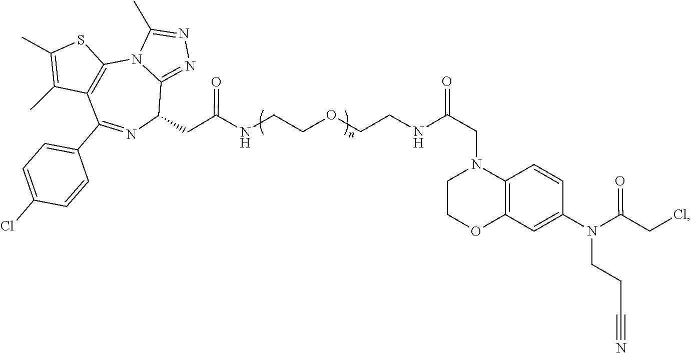

wherein n is an integer from 1 to 8.

In embodiments, n is 1. In embodiments, n is 2. In embodiments, n is 3. In embodiments, n is 4. In embodiments, n is 5. In embodiments, n is 6. In embodiments, n is 7. In embodiments, n is 8.

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in an aspect, embodiment, example, table, figure, or claim).

In embodiments, the compound is a compound described herein, or a pharmaceutically acceptable salt thereof.

### III. Pharmaceutical Compositions

In an aspect, provided herein is a pharmaceutical composition including a compound as described herein and a pharmaceutically acceptable excipient. In embodiments, the compound as described herein is included in a therapeutically effective amount.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g., therapeutic agent). In embodiments of the pharmaceutical compositions, the pharmaceutical composition includes a second agent (e.g., therapeutic agent) in a therapeutically effective amount. In embodiments of the pharmaceutical compositions, the second agent is an agent for treating cancer. In embodiments of the pharmaceutical compositions, the second agent is an agent for treating an inflammatory disease. In embodiments of the pharmaceutical compositions, the second agent is an agent for treating an autoimmune disease. In embodiments, the administering does not include administration of any active agent other than the recited active agent (e.g., a compound described herein).

### IV. Methods of Use

In an aspect, provided herein is a method of treating a disease in a subject in need thereof, the method including administering a therapeutically effective amount of FEM1B Cys 186 covalent inhibitor.

In embodiments, the disease is cancer, neurodegenerative disease, mitochondrial disease, obesity, Huntington's disease or diabetes. In embodiments, the disease is cancer, obesity, Huntington's disease or diabetes. In embodiments, the disease is cancer, neurodegenerative disease, mitochondrial disease, and diabetes. In embodiments, the disease is cancer. In embodiments, the disease is a neurodegenerative disease. In embodiments, the disease is a mitochondrial disease. In embodiments, the disease is obesity. In embodiments, the disease is Huntington's disease. In embodiments, the disease is diabetes.

In embodiments, the cancer is metastatic lung cancer, neuroblastoma, colon cancer, leukemia, prostate cancer, renal cancer, or multiple myeloma. In embodiments, the cancer is a metastatic lung cancer. In embodiments, the cancer is a neuroblastoma. In embodiments, the cancer is a colon cancer. In embodiments, the cancer is leukemia. In embodiments, the cancer is a prostate cancer. In embodiments, the cancer is a renal cancer. In embodiments, the cancer is a multiple myeloma.

In embodiments, the cancer is metastatic lung cancer, neuroblastoma, colon cancer, leukemia, prostate cancer, or renal cancer. In embodiments, the cancer is metastatic lung cancer, neuroblastoma, colon cancer, or renal cancer. In embodiments, the cancer is leukemia or prostate cancer. In embodiments, the cancer is a metastatic lung cancer. In embodiments, the cancer is a neuroblastoma. In embodiments, the cancer is a colon cancer. In embodiments, the cancer is leukemia. In embodiments, the cancer is a prostate cancer. In embodiments, the cancer is a renal cancer.

In embodiments, the renal cancer is a KEAP1 mutated renal cancer. In embodiments, the prostate cancer is a castration-resistant prostate cancer.

In embodiments, the diabetes is a type-H diabetes.

In embodiments, the neurodegenerative disease is Huntington Disease. In embodiments, the neurodegenerative disease is Alzheimer Disease. In embodiments, the neurodegenerative disease is Parkinson's Disease. In embodiments, the neurodegenerative disease is frontotemporal dementia. In embodiments, the method includes reducing protein aggregates (e.g., in the brain). In embodiments, the method includes reducing TDP-43 aggregates (e.g., in the brain). In embodiments, the neurodegenerative disease is amyotrophic lateral sclerosis. In embodiments, the neurodegenerative disease is chronic traumatic encephalopathy. In embodiments, the neurodegenerative disease is traumatic brain injury (e.g., concussion).

In embodiments, the metabolic disease is diabetes. In embodiments, the metabolic disease is type I diabetes. In embodiments, the metabolic disease is type H diabetes. In embodiments, the metabolic disease is obesity. In embodiments, the metabolic disease is metabolic syndrome. In embodiments, the metabolic disease is a mitochondrial disease (e.g., dysfunction of mitochondria or aberrant mitochondrial function).

“FEM1B Cys 186 covalent inhibitor” is a compound as described herein, capable of covalently binding to Cys 186 on the FEM1B protein. Thus, a disease associated with FEM1B activity or function (e.g., signaling pathway activity) or a FEM1B associated disease (e.g., cancer, a neurodegenerative disease, or a mitochondrial disease), may be treated with a FEM1B Cys 186 covalent inhibitor, in the instance where increased FEM1B activity or function (e.g., signaling pathway activity) causes the disease.

The term “FEM1B” includes any recombinant or naturally-occurring form of FEM1B or variants thereof that maintain FEM1B function or activity (e.g., within at least 30%, 40%, 50%, 60%, 70%, 80%, 90%, 95%, or 100% function or activity compared to wildtype FEM1B). In embodiments, FEM1B is encoded by the FEM1B gene. In embodiments, FEM1B has the amino acid sequence set forth in or corresponding to Entrez NP 056137, UniProt Q9UK73, or RefSeq (protein) NM 015322.5. In embodiments, FEM1B has the sequence (also referred to herein as the FEM1B reference sequence):

(SEQ ID NO: 1)
MEGLAGYVYKAASEGKVLTLAALLLNRSES
DIRYLLGYVSQQGGQRSTPLIIAARNGHAK
VVRLLLEHYRVQTQQTGTVRFDGYVIDGAT
ALWCAAGAGHFEVVKLLVSHGANVNHTTVT
NSTPLRAACFDGRLDIVKYLVENNANISIA
NKYDNTCLMIAAYKGHTDVVRYLLEQRADP
NAKAHCGATALHFAAEAGHIDIVKELIKWR
AAIVVNGHGMTPLKVAAESCKADVVELLLS
HADCDRRSRIEALELLGASFANDRENYDII
KTYHYLYLAMLERFQDGDNILEKEVLPPIH
AYGNRTECRNPQELESIRQDRDALHMEGLI
VRERILGADNIDVSHPIIYRGAVYADNMEF
EQCIKLWLHALHLRQKGNRNTHKDLLRFAQ
VFSQMIHLNETVKAPDIECVLRCSVLEIEQ
SMNRVKNISDADVHNAMDNYECNLYTFLYL
VCISTKTQCSEEDQCKINKQIYNLIHLDPR
TREGFTLLHLAVNSNTPVDDFHTNDVCSFP
NALVTKLLLDCGAEVNAVDNEGNSALHIIV
QYNRPISDFLTLHSIIISLVEAGAHTDMTN
KQNKTPLDKSTTGVSEILLKTQMKMSLKCL
AARAVRANDINYQDQIPRTLEEFVGFH.

Thus, “an amino acid corresponding to Cys 186” refers to a cysteine amino acid within a FEM1B protein that may be at a numbered amino acid position different from position 186 in the above reference sequence due to difference in the sequence number of the FEM1B protein relative to the above reference sequence (e.g., a homolog of the FEM1B reference sequence above), but wherein that cysteine amino acid within the FEM1B protein at a numbered position different from position 186 is functionally equivalent to Cys 186 in the above reference sequence.

The term “Brd4” is used in accordance with its plain and ordinary meaning, and refers to bromodomain-containing protein 4 protein (including homologs, isoforms, and functional fragments thereof). In embodiments, Brd4 is a member of the BET (bromodomain and extra terminal domain) family, which also includes Brd2, Brd3, and Brdt. Brd4, similar to other BET family members, typically contains two bromodomains that recognize acetylated lysine residues. The term includes any recombinant or naturally-occurring form of Brd4 protein or variants thereof that maintain Brd4 activity (e.g., within at least 30%, 40%, 50%, 60%, 70%, 80%, 90%, 95%, or 100% activity compared to wildtype Brd4). In embodiments, the Brd4 protein encoded by the Brd4 gene has the amino acid sequence set forth in or corresponding to UniProt 060885, or RefSeq (protein) NP 490597. In embodiments, the Brd4 gene has the nucleic acid sequence set forth in RefSeq (mRNA) NM 058243. In embodiments, the amino acid sequence or nucleic acid sequence is the sequence known at the time of filing of the present application. In embodiments, the sequence corresponds to GI:19718731. In embodiments, the sequence corresponds to NP 490597.1. In embodiments, the sequence corresponds to NM 058243.2. In embodiments, the sequence corresponds to GI: 112789559.

The term “Kelch-like ECH-associated protein 1” or “KEAP1” is used in accordance with its plain and ordinary meaning, and refers to a protein (including homologs, isoforms, and functional fragments thereof) that regulates the response to oxidative stress. The term includes any recombinant or naturally-occurring form of KEAP1 protein or variants thereof that maintain KEAP1 activity (e.g., within at least 30%, 40%, 50%, 60%, 70%, 80%, 90%, 95%, or 100% activity compared to wildtype KEAP1). In embodiments, the KEAP1 protein encoded by the KEAP1 gene has the amino acid sequence set forth in or corresponding to UniProt Q14145, RefSeq (protein) NP_036421, or RefSeq (protein) NP 987096. In embodiments, the KEAP1 gene has the nucleic acid sequence set forth in RefSeq (mRNA) NM_012289 or RefSeq (mRNA) NM_203500. In embodiments, the amino acid sequence or nucleic acid sequence is the sequence known at the time of filing of the present application.

The term “folliculin-interacting protein 1” or “FNIP1” is used in accordance with its plain and ordinary meaning, and refers to a protein (including homologs, isoforms, and functional fragments thereof) involved in the cellular response to amino acid availability by refulating mTORC1 signalling cascade. The term includes any recombinant or naturally-occurring form of FNIP1 protein or variants thereof that maintain FNIP1 activity (e.g., within at least 30%, 40%, 50%, 60%, 70%, 80%, 90%, 95%, or 100% activity compared to wildtype FNIP1). In embodiments, the FNIP1 protein encoded by the FNIP1 gene has the amino acid sequence set forth in or corresponding to UniProt Q8TF40, RefSeq (protein) NP 001008738, RefSeq (protein) NP 001333042, RefSeq (protein) NP_001333043, or RefSeq (protein) NP_588613. In embodiments, the FNIP1 gene has the nucleic acid sequence set forth in RefSeq (mRNA) NM 133372, RefSeq (mRNA) NM 001008738, RefSeq (mRNA) NM_001346113, or RefSeq (mRNA) NM 001346114. In embodiments, the amino acid sequence or nucleic acid sequence is the sequence known at the time of filing of the present application.

The term “K-ras” is used in accordance with its plain and ordinary meaning, and refers to a protein (including homologs, isoforms, and functional fragments thereof) involved in the regulation of cell proliferation. The term includes any recombinant or naturally-occurring form of K-ras protein or variants thereof that maintain K-ras activity (e.g., within at least 30%, 40%, 50%, 60%, 70%, 80%, 90%, 95%, or 100% activity compared to wildtype K-ras). In embodiments, the K-ras protein encoded by the K-ras gene has the amino acid sequence set forth in or corresponding to UniProt P01116, RefSeq (protein) NP 004976, RefSeq (protein) NP 203524, RefSeq (protein) NP 001356715, RefSeq (protein) NP_001356716, or RefSeq (protein) NP 004976.2. In embodiments, the K-ras gene has the nucleic acid sequence set forth in RefSeq (mRNA) NM_004985, RefSeq (mRNA) NM_033360, RefSeq (mRNA) NM_001369786, or RefSeq (mRNA) NM_001369787. In embodiments, the amino acid sequence or nucleic acid sequence is the sequence known at the time of filing of the present application.

The term “Bruton's tyrosine kinase” or “BTK” is used in accordance with its plain and ordinary meaning, and refers to a protein (including homologs, isoforms, and functional fragments thereof) that plays a role in B cell development. The term includes any recombinant or naturally-occurring form of BTK protein or variants thereof that maintain BTK activity (e.g., within at least 30%, 40%, 50%, 60%, 70%, 80%, 90%, 95%, or 100% activity compared to wildtype BTK). In embodiments, the BTK protein encoded by the BTK gene has the amino acid sequence set forth in or corresponding to UniProt Q06187, RefSeq (protein) NP 000052, RefSeq (protein) NP_001274273, or RefSeq (protein) NP_001274274. In embodiments, the BTK gene has the nucleic acid sequence set forth in RefSeq (mRNA) NM_001287345, RefSeq (mRNA) NM_000061, or RefSeq (mRNA) NM 001287344. In embodiments, the amino acid sequence or nucleic acid sequence is the sequence known at the time of filing of the present application.

The term “androgen receptor” or “AR” or “NR3C4” is used in accordance with its plain and ordinary meaning, and refers to a nuclear receptor (including homologs, isoforms, and functional fragments thereof) that is activated by binding any of the androgenic hormones. The term includes any recombinant or naturally-occurring form of AR protein or variants thereof that maintain AR activity (e.g., within at least 30%, 40%, 50%, 60%, 70%, 80%, 90%, 95%, or 100% activity compared to wildtype AR). In embodiments, the AR protein encoded by the AR gene has the amino acid sequence set forth in or corresponding to UniProt P10275, UniProt Q9NUA2, RefSeq (protein) NP_000035.2, RefSeq (protein) NP_001011645.1, RefSeq (protein) NP 001334990, RefSeq (protein) NP 001334992, or RefSeq (protein) NP 001334993. In embodiments, the AR gene has the nucleic acid sequence set forth in RefSeq (mRNA) NM 001011645, RefSeq (mRNA) NM_000044, RefSeq (mRNA) NM 001348061, RefSeq (mRNA) NM_001348063, or RefSeq (mRNA) NM_001348064. In embodiments, the amino acid sequence or nucleic acid sequence is the sequence known at the time of filing of the present application.

The term “MYC” is used in accordance with its plain and ordinary meaning, and refers to a protein (including homologs, isoforms, and functional fragments thereof) that plays a role in cell cycle progression, apoptosis, and cellular transformation. The term includes any recombinant or naturally-occurring form of MYC protein or variants thereof that maintain MYC activity (e.g., within at least 30%, 40%, 50%, 60%, 70%, 80%, 90%, 95%, or 100% activity compared to wildtype MYC). In embodiments, the MYC protein encoded by the MYC gene has the amino acid sequence set forth in or corresponding to UniProt P01106, RefSeq (protein) NP 002458, or RefSeq (protein) NP 001341799. In embodiments, the MYC gene has the nucleic acid sequence set forth in RefSeq (mRNA) NM_002467 or RefSeq (mRNA) NM_001354870. In embodiments, the amino acid sequence or nucleic acid sequence is the sequence known at the time of filing of the present application.

The term “N-MYC” is used in accordance with its plain and ordinary meaning, and refers to a protein (including homologs, isoforms, and functional fragments thereof) that plays a role in brain development. The term includes any recombinant or naturally-occurring form of N-MYC protein or variants thereof that maintain N-MYC activity (e.g., within at least 30%, 40%, 50%, 60%, 70%, 80%, 90%, 95%, or 100% activity compared to wildtype N-MYC). In embodiments, the N-MYC protein encoded by the MYCN gene has the amino acid sequence set forth in or corresponding to UniProt P04198, RefSeq (protein) NP_001280157, RefSeq (protein) NP_001280160, RefSeq (protein) NP 001280162, or RefSeq (protein) NP 005369. In embodiments, the MYCN gene has the nucleic acid sequence set forth in RefSeq (mRNA) NM 005378, RefSeq (mRNA) NM 001293228, RefSeq (mRNA) NM_001293231, or RefSeq (mRNA) NM_001293233. In embodiments, the amino acid sequence or nucleic acid sequence is the sequence known at the time of filing of the present application.

The term “beta-catenin” is used in accordance with its plain and ordinary meaning, and refers to a protein (including homologs, isoforms, and functional fragments thereof) that is involved in regulation and coordination of cell-cell adhesion and gene transcription. The term includes any recombinant or naturally-occurring form of beta-catenin protein or variants thereof that maintain beta-catenin activity (e.g., within at least 30%, 40%, 50%, 60%, 70%, 80%, 90%, 95%, or 100% activity compared to wildtype beta-catenin). In embodiments, the beta-catenin protein encoded by the CTNNB1 gene has the amino acid sequence set forth in or corresponding to UniProt P35222, RefSeq (protein) NP_001091679, RefSeq (protein) NP_001091680, RefSeq (protein) NP_001317658, or RefSeq (protein) NP_001895. In embodiments, the CTNNB1 gene has the nucleic acid sequence set forth in RefSeq (mRNA) NM_001098209, RefSeq (mRNA) NM_001098210, RefSeq (mRNA)NM_001904, or RefSeq (mRNA) NM_001330729. In embodiments, the amino acid sequence or nucleic acid sequence is the sequence known at the time of filing of the present application.

The term “Huntingtin” or “HTT” is used in accordance with its plain and ordinary meaning, and refers to a protein (including homologs, isoforms, and functional fragments thereof) that is involved in axonal transport. The term includes any recombinant or naturally-occurring form of HTT protein or variants thereof that maintain HTT activity (e.g., within at least 30%, 40%, 50%, 60%, 70%, 80%, 90%, 95%, or 100% activity compared to wildtype HT). In embodiments, the HTT protein encoded by the HTT gene has the amino acid sequence set forth in or corresponding to UniProt P42858 or RefSeq (protein) NP 002102. In embodiments, the HTT gene has the nucleic acid sequence set forth in RefSeq (mRNA) RefSeq (mRNA) NM 002111. In embodiments, the amino acid sequence or nucleic acid sequence is the sequence known at the time of filing of the present application.

In an aspect, provided herein is a method of treating a disease in a subject in need thereof, the method including administering a therapeutically effective amount of a compound having the structure: FCIM-L³-R³.

FCIM is a FEM1B Cys 186 covalent inhibitor moiety.

R³ is a target protein binding moiety.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is as described herein including in embodiments. L³ is as described herein including in embodiments. R¹⁰³ is as described herein including in embodiments. FCIM-L³-R³ is a compound wherein FCIM is a FEM1B Cys 186 covalent inhibitor moiety.

The term “covalent cysteine modifier moiety” as used herein refers to a monovalent electrophilic moiety that is able to measurably bind to a cysteine amino acid. As used herein, a “Cys 186 covalent inhibitor moiety” is a “covalent cysteine modifier moiety” that is able to measurably bind to an amino acid that corresponds to Cys 186 in an FEM1B protein. In embodiments, the monovalent electrophilic moiety is able to measurably bind to a cysteine amino acid of FEM1B. In embodiments, the covalent cysteine modifier moiety binds via an irreversible covalent bond. In embodiments, the covalent cysteine modifier moiety binds via a reversible covalent bond. In embodiments, the covalent cysteine modifier moiety binds via a non-covalent bond. In embodiments, the covalent cysteine modifier moiety is capable of binding with a Kd of less than about 1 mM, 500 μM, 100 μM, 50 μM, 10 μM, 5 μM, 1 μM, 500 nM, 250 nM, 100 nM, 75 nM, 50 nM, 25 nM, 15 nM, 10 nM, 5 nM, 1 nM, or about 0.1 nM.

In an aspect, provided herein is a FEM1B protein including an amino acid corresponding to Cys 186. Amino acid corresponding to Cys 186 is covalently bound to a (i) FEM1B Cys 186 covalent inhibitor, or (ii) a compound having the structure: FCIM-L³-R³.

FCIM is a FEM1B Cys 186 covalent inhibitor moiety.

R³ is a target protein binding moiety.

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹⁰³ is independently hydrogen, halogen, —CCl₃, —CBr₃, —CF₃, —CI₃, —CHCl₂, —CHBr₂, —CHF₂, —CHI₂, —CH₂CL, —CH₂Br, —CH₂F, -CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

FCIM is covalently bound to the Cys 186.

In embodiments, R¹ contacts a FEM1B protein amino acid corresponding to FEM1B His185, Cys186, Gly187, Gly217, Asn216, His218, Asn340, and Ile341.

### V. Embodiments

Embodiment P1. A compound having the formula:

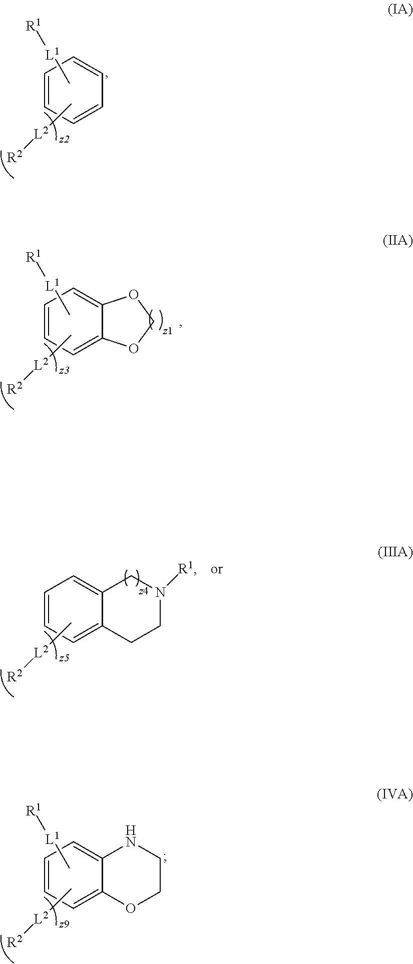

or a pharmaceutically acceptable salt thereof, wherein:

R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₃, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰¹ is independently hydrogen, oxo, halogen, —CCl₃, —CBr₃, —CF₃, —CI₃, —CHCl₂, —CHBr₂, —CHF₂, —CHI₂,—C₁HC, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is an electrophilic moiety;

z1 is 1 or 2;

z2 is 0 to 5;

z3 is 0 to 3;

z4 is 0 or 1; and

z5 and z9 are each independently an integer from 0 to 4.

Embodiment P2. The compound of embodiment P1, or a pharmaceutically acceptable salt thereof, wherein:

R² is independently halogen, —CCl₃, —CBr₃, —CF₃, —CI₃, —CN, —OH, —NH₂, —COOH, substituted or unsubstituted alkyl, or substituted or unsubstituted heteroalkyl;

L² is independently a bond, —N(R¹⁰²)—, —C(O)—, substituted or unsubstituted alkylene, or substituted or unsubstituted heteroalkylene;

R¹⁰ is independently hydrogen or unsubstituted alkyl;

L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;

R¹⁰¹ is independently hydrogen, —OH, —NH₂, —COOH, —CONH₂, substituted or unsubstituted alkyl, substituted or unsubstituted aryl, or substituted or unsubstituted heteroaryl;

R¹ is:

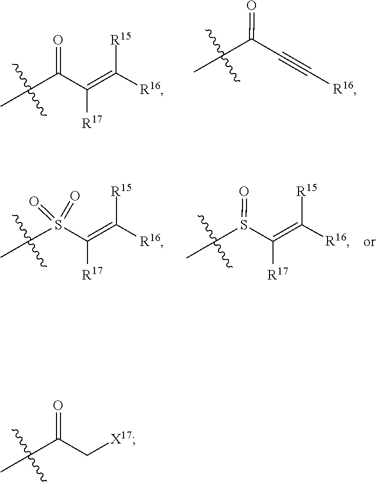

R¹⁵, R¹⁶, and R¹⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCHI₁, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

and X¹⁷ is halogen.

Embodiment P3. The compound of embodiment P1 or P2, or a pharmaceutically acceptable salt thereof, wherein:

R² is independently halogen, —CF₃, unsubstituted C₁-C₃ alkyl or unsubstituted 2 to 3 membered heteroalkyl;

L² is independently a bond;

L¹ is —N(R¹⁰¹)—; and

R¹⁰¹ is independently hydrogen, substituted or unsubstituted alkyl, substituted or unsubstituted aryl, or substituted or unsubstituted heteroaryl.

Embodiment P4. The compound of any one of embodiments P1-P3, or a pharmaceutically acceptable salt thereof, wherein:

R² is independently —Cl, —Br, —F, —CF₃, —CH₃, —OCH₃, or —OCH₂CH₃;

L² is independently a bond;

L¹ is —N(R¹⁰¹)—;

R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;

R¹ is:

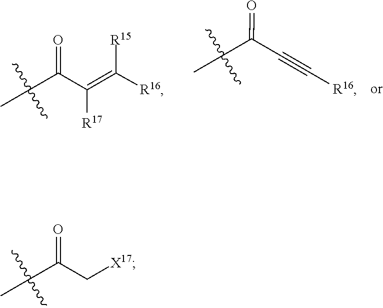

R¹⁵, R¹⁶, and R¹⁷ are independently hydrogen; and

X¹⁷ is halogen.

Embodiment P5. The compound of any one of embodiments P1-P4, or a pharmaceutically acceptable salt thereof, wherein:

L¹ is —N(R¹⁰¹)—;

R¹⁰¹ is independently hydrogen, —CH₂CH₂CN,

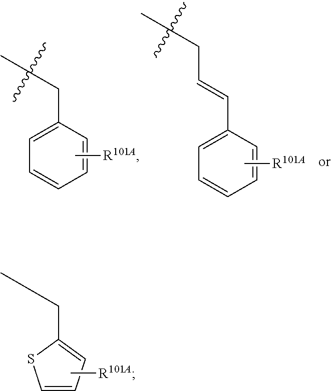

R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is:

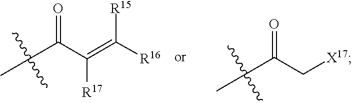

R¹⁵, R¹⁶, and R¹⁷ are independently hydrogen; and

X¹⁷ is halogen.

Embodiment P6. The compound of any one of embodiments P1-P5, or a pharmaceutically acceptable salt thereof, wherein:

L¹ is —N(R¹⁰¹)—; and

R¹⁰¹ is independently hydrogen, —CH₂CH₂CN,

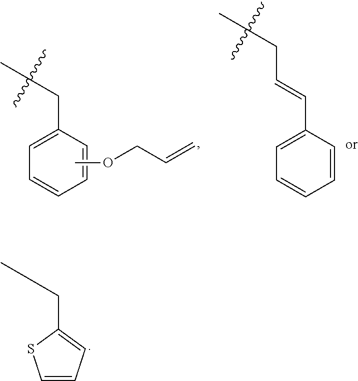

Embodiment P7. A compound having the formula:

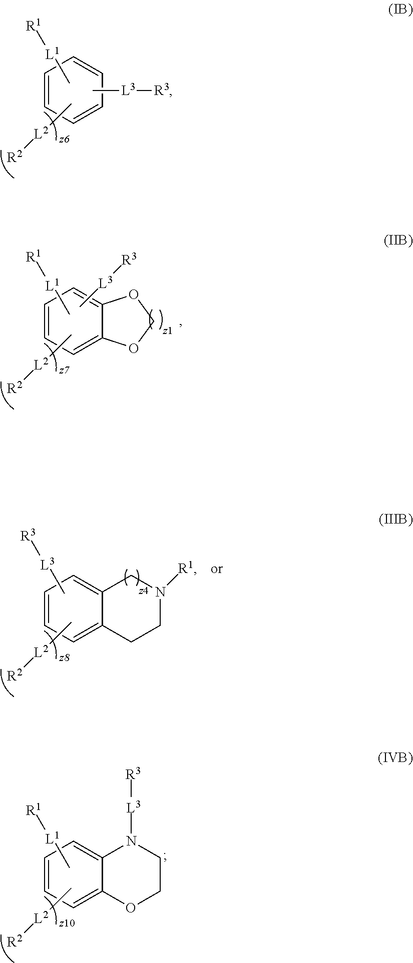

or a pharmaceutically acceptable salt thereof, wherein:

R² is independently halogen —CCl₃, —CBr₃, —CF₃, —CI₃, —CHCl₂, —CHBr₂, —CHF₂, —CHI₂, —CH₂Cl, —CH₂Br, —CH₂F, -CH₂I, —CN, —OH, —NH₂, —COOH, —CONH₂, —NO₂, —SH, —SO₃H, —SO₄H, —SO₂NH₂, —NHNH₂, —NH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is independently a bond, —S(O)₂—, —N(R¹⁰²)—, —O—, —S—, —C(O)—, —C(O)N(R¹⁰²)—, —N(R¹⁰²)C(O), —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰ is independently hydrogen, oxo, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰¹ is independently hydrogen, oxo, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is an electrophilic moiety;

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-.

R¹⁰³ is independently hydrogen, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R³ is a target protein binding moiety;

z1 is 1 or 2;

z4 is 0 or 1;

z6 is 0 to 4;

z7 is 0 to 2; and

z8 and z10 are each independently an integer from 0 to 3.

Embodiment P8. The compound of embodiment P7, or a pharmaceutically acceptable salt thereof, wherein:

R² is independently halogen, —CCl₃, —CBr₃, —CF₃, —CI₃, —CN, —OH, —NH₂, —COOH, substituted or unsubstituted alkyl, or substituted or unsubstituted heteroalkyl;

L² is independently a bond, —N(R¹⁰²)—, —C(O)—, substituted or unsubstituted alkylene, or substituted or unsubstituted heteroalkylene;

R¹⁰² is independently hydrogen or unsubstituted alkyl;

L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;

R¹⁰¹ is independently hydrogen, —OH, —NH₂, —COOH, —CONH₂, substituted or unsubstituted alkyl, substituted or unsubstituted aryl, or substituted or unsubstituted heteroaryl;

R¹ is:

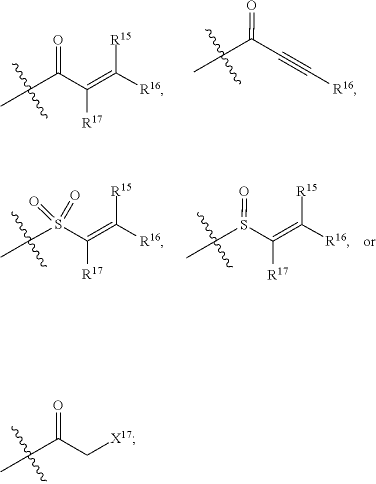

R¹⁵, R¹⁶, and R¹⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X¹⁷ is halogen;

L³ is a bond, —N(R¹⁰³)—, —O—, —S—, —C(O)—, substituted or unsubstituted alkylene, or substituted or unsubstituted heteroalkylene;

R¹⁰ is independently hydrogen, —OH, or substituted or unsubstituted alkyl; and

R³ is a target protein binding moiety.

Embodiment P9. The compound of embodiment P7 or P8, or a pharmaceutically acceptable salt thereof, wherein:

R² is independently halogen, —CF₃, unsubstituted C₁-C₃ alkyl or unsubstituted 2 to 3 membered heteroalkyl;

L² is independently a bond;

L¹ is —N(R¹⁰¹)—;

R¹⁰¹ is independently hydrogen, substituted or unsubstituted alkyl, substituted or unsubstituted aryl, or substituted or unsubstituted heteroaryl;

L³ is a bond, substituted or unsubstituted C₁-C₆ alkylene, or substituted or unsubstituted 2 to 6 membered heteroalkylene; and

R³ is a target protein binding moiety.

Embodiment P10. The compound of anyone of embodiments P7-P9, or a pharmaceutically acceptable salt thereof, wherein:

R² is independently —Cl, —Br, —F, —CF₃, —CH₃, —OCH₃, or —OCH₂CH₃;

L² is independently a bond;

L¹ is —N(R¹⁰¹)—;

R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;

R¹ is:

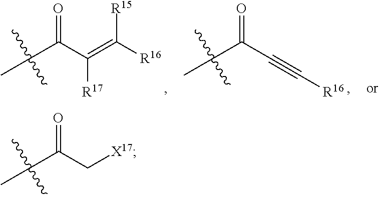

R¹⁵, R¹⁶, and R¹⁷ are independently hydrogen;

X¹⁷ is halogen;

L³ is a bond or substituted or unsubstituted 2 to 6 membered heteroalkylene; and

R³ is a target protein binding moiety.

Embodiment P11. The compound of anyone of embodiments P7-P10, or a pharmaceutically acceptable salt thereof, wherein:

L¹ is —N(R¹⁰¹)—;

R¹⁰¹ is independently hydrogen, —CH₂CH₂CN,

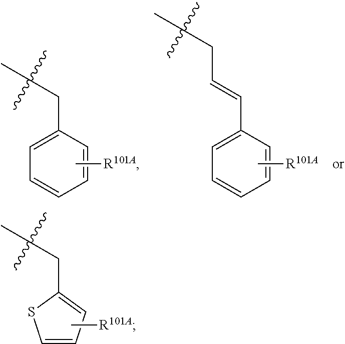

R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is:

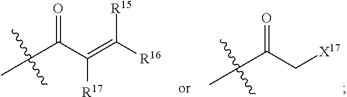

R¹⁵, R¹⁶, and R¹⁷ are independently hydrogen;

X¹⁷ is halogen;

L³ is a bond or substituted or unsubstituted 2 to 6 membered heteroalkylene; and

R³ is a target protein binding moiety.

Embodiment P12. The compound of anyone of embodiments P7-P11, or a pharmaceutically acceptable salt thereof, wherein:

L¹ is —N(R¹⁰¹)—;

R¹⁰¹ is independently hydrogen, —CH₂CH₂CN,

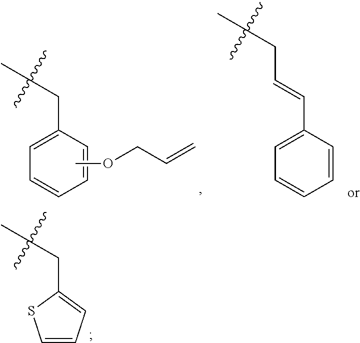

L³ is a bond or substituted or unsubstituted 2 to 6 membered heteroalkylene; and

R³ is a target protein binding moiety.

Embodiment P13. The compound of anyone of embodiments P7-P12, or a pharmaceutically acceptable salt thereof, wherein R³ is a Brd4 binding moiety.

Embodiment P14. The compound of anyone of embodiments P7-P12, or a pharmaceutically acceptable salt thereof, wherein R³ is a K-ras binding moiety.

Embodiment P15. The compound of anyone of embodiments P7-P12, or a pharmaceutically acceptable salt thereof, wherein R³ is a Bruton's tyrosine kinase (BTK) binding moiety.

Embodiment P16. The compound of anyone of embodiments P7-P12, or a pharmaceutically acceptable salt thereof, wherein R³ is an androgen receptor (AR) binding moiety.

Embodiment P17. The compound of anyone of embodiments P7-P12, or a pharmaceutically acceptable salt thereof, wherein R³ is a MYC protein binding moiety.

Embodiment P18. The compound of anyone of embodiments P7-P12, or a pharmaceutically acceptable salt thereof, wherein R³ is an N-MYC protein binding moiety.

Embodiment P19. The compound of anyone of embodiments P7-P12, or a pharmaceutically acceptable salt thereof, wherein R³ is a beta-catenin protein binding moiety.

Embodiment P20. The compound of anyone of embodiments P7-P12, or a pharmaceutically acceptable salt thereof, wherein R³ is a huntingtin (HT) protein binding moiety.

Embodiment P21. A compound selected from a group consisting of:

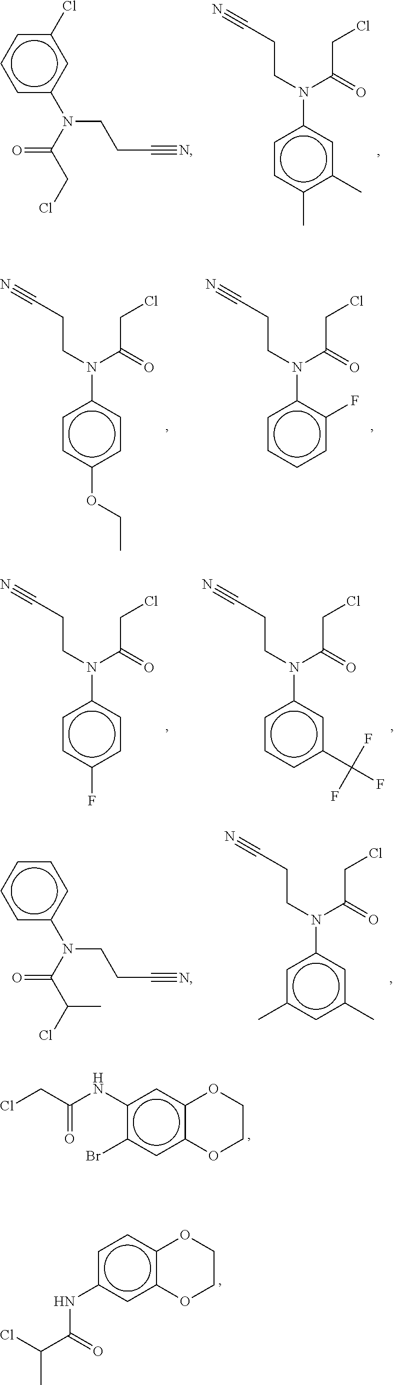

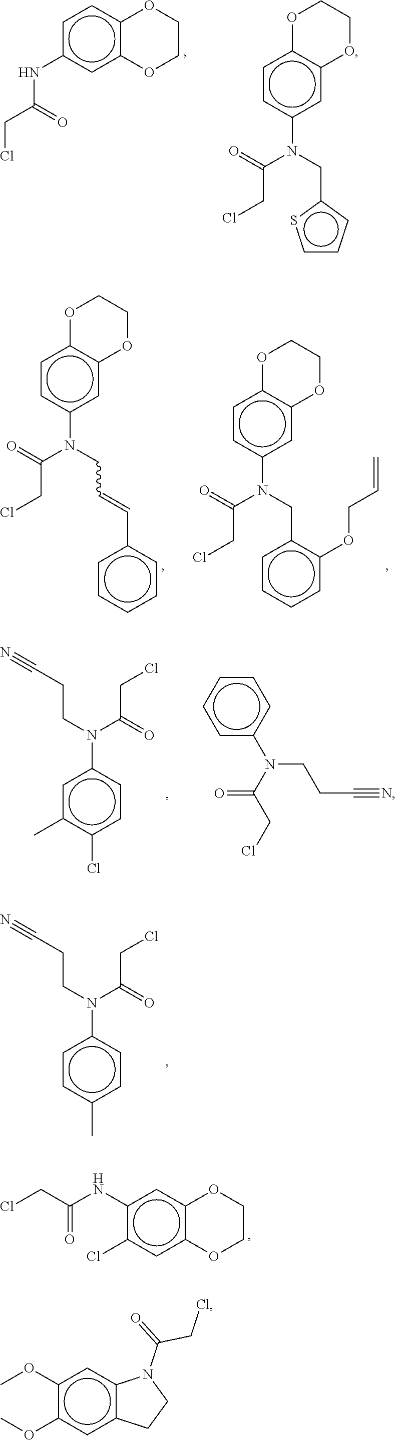

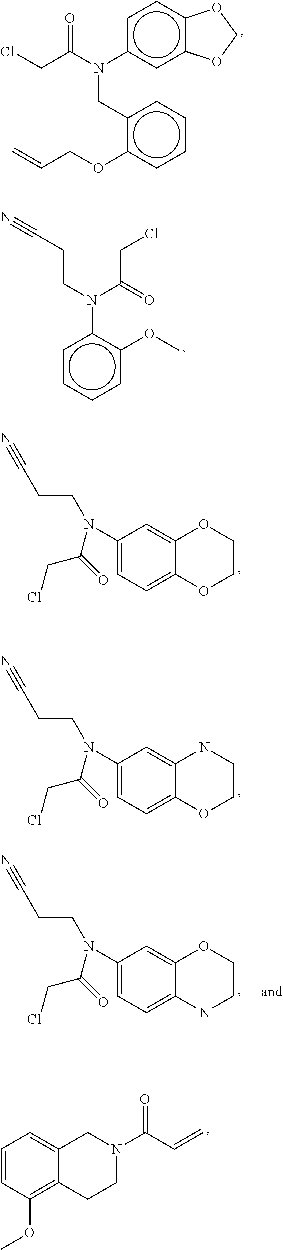

or a pharmaceutically acceptable salt thereof.

Embodiment P22. A pharmaceutical composition comprising the compound of any one of embodiments P1 to P21 and a pharmaceutically acceptable excipient.

Embodiment P23. A method of treating a disease in a subject in need thereof, the method comprising administering a therapeutically effective amount of FEM1B Cys 186 covalent inhibitor.

Embodiment P24. The method of embodiment P23, wherein the disease is cancer, obesity, Huntington's disease or diabetes.

Embodiment P25. The method of embodiment P24, wherein the cancer is a metastatic lung cancer, neuroblastoma, colon cancer, or renal cancer.

Embodiment P26. The method of embodiment P25, wherein the renal cancer is a KEAP1 mutated renal cancer.

Embodiment P27. The method of embodiment P24, wherein the diabetes is a type-II diabetes.

Embodiment P28. A method of treating a disease in a subject in need thereof, the method comprising administering a therapeutically effective amount of a compound having the structure: FCIM-L³-R³, wherein

FCIM is a FEM1B Cys 186 covalent inhibitor moiety,

R³ is a target protein binding moiety;

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;

R¹⁰³ is independently hydrogen, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

Embodiment P29. The method of embodiment P28, wherein the disease is cancer, neurodegenerative disease, mitochondrial disease, and diabetes.

Embodiment P30. The method of embodiment P29, wherein the cancer is a leukemia or prostate cancer.

Embodiment P31. The method of embodiment P30, wherein the prostate cancer is a castration-resistant prostate cancer.

Embodiment P32. A FEM1B protein comprising an amino acid corresponding to Cys 186, wherein said amino acid corresponding to Cys 186 is covalently bound to a (i) FEM1B Cys 186 covalent inhibitor, or (ii) a compound having the structure: FCIM-L³-R³, wherein

FCIM is a FEM1B Cys 186 covalent inhibitor moiety,

R³ is a target protein binding moiety;

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;

R¹⁰³ is independently hydrogen, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

wherein the FCIM is covalently bound to said Cys 186.

### VI. ADDITIONAL EMBODIMENTS

Embodiment 1. A compound having the formula:

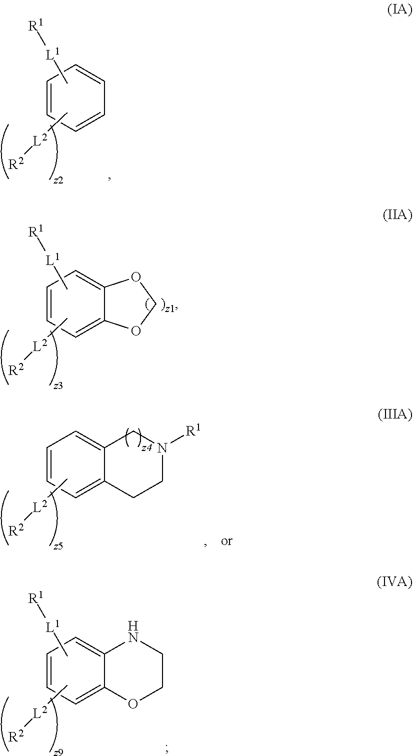

or a pharmaceutically acceptable salt thereof, wherein:

R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰¹ is independently hydrogen, oxo, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is an electrophilic moiety;

z1 is 1 or 2;

z2 is 0 to 5;

z3 is 0 to 3;

z4 is 0 or 1; and

z5 and z9 are each independently an integer from 0 to 4.

Embodiment 2. The compound of embodiment 1, or a pharmaceutically acceptable salt thereof, wherein:

R² is independently halogen, —CCl₃, —CBr₃, —CF₃, —CI₃, —CN, —OH, —NH₂, —COOH, substituted or unsubstituted alkyl, or substituted or unsubstituted heteroalkyl;

L² is independently a bond, —N(R¹⁰²)—, —C(O)—, substituted or unsubstituted alkylene, or substituted or unsubstituted heteroalkylene;

R¹⁰ is independently hydrogen or unsubstituted alkyl;

L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;

R¹⁰¹ is independently hydrogen, —OH, —NH₂, —COOH, —CONH₂, substituted or unsubstituted alkyl, substituted or unsubstituted aryl, or substituted or unsubstituted heteroaryl;

R¹ is:

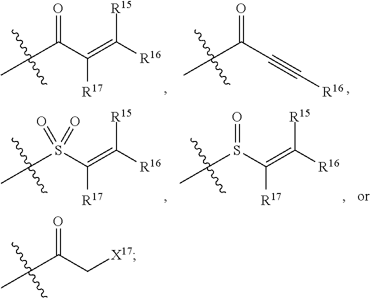

R¹⁵, R¹⁶, and R¹⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

X¹⁷ is halogen.

Embodiment 3. The compound of embodiment 1 or 2, or a pharmaceutically acceptable salt thereof, wherein:

R² is independently halogen, —CF₃, unsubstituted C₁-C₃ alkyl, or unsubstituted 2 to 3 membered heteroalkyl;

L² is independently a bond;

L¹ is —N(R¹⁰¹)—; and

R¹⁰¹ is independently hydrogen, substituted or unsubstituted alkyl, substituted or unsubstituted aryl, or substituted or unsubstituted heteroaryl.

Embodiment 4. The compound of any one of embodiments 1 to 3, or a pharmaceutically acceptable salt thereof, wherein:

R² is independently —Cl, —Br, —F, —CF₃, —CH₃, —OCH₃, or —OCH₂CH₃;

L² is independently a bond;

L¹ is —N(R¹⁰¹)—;

R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;

R¹ is:

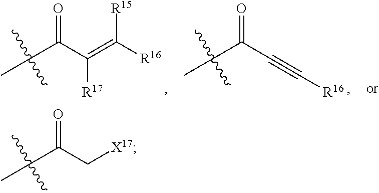

R¹⁵, R¹⁶, and R¹⁷ are independently hydrogen; and

X¹⁷ is halogen.

Embodiment 5. The compound of any one of embodiments 1 to 4, or a pharmaceutically acceptable salt thereof, wherein:

L¹ is —N(R¹⁰¹)—;

R¹⁰¹ is independently hydrogen, —CH₂CH₂CN,

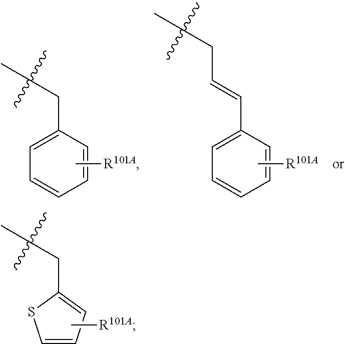

R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is:

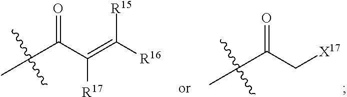

R¹⁵, R¹⁶, and R¹⁷ are independently hydrogen; and

X¹⁷ is halogen.

Embodiment 6. The compound of any one of embodiments 1 to 5, or a pharmaceutically acceptable salt thereof, wherein:

L¹ is —N(R¹⁰¹)—; and

R¹⁰¹ is independently hydrogen, —CH₂CH₂CN,

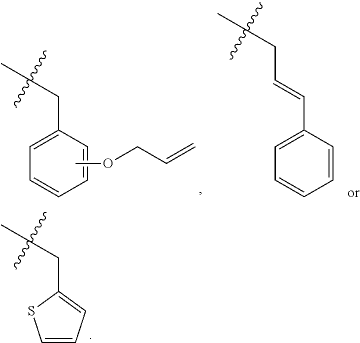

Embodiment 7. A compound having the formula:

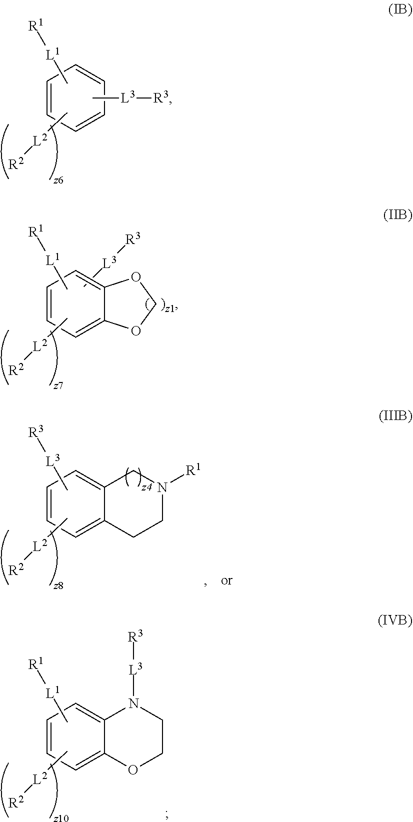

or a pharmaceutically acceptable salt thereof, wherein:

R² is independently halogen —CCl₃, —CBr₃, —CF₃, —CI₃, —CHCl₂, —CHBr₂, —CHF₂, —CHI₂, —CH₂Cl, —CH₂Br, —CH₂, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂I, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is an electrophilic moiety;

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;

R¹⁰² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R³ is a target protein binding moiety;

z1 is 1 or 2;

z4 is 0 or 1;

z6 is 0 to 4;

z7 is 0 to 2; and

z8 and z10 are each independently an integer from 0 to 3.

Embodiment 8. The compound of embodiment 7, or a pharmaceutically acceptable salt thereof, wherein:

R² is independently halogen, —CCl₃, —CBr₃, —CF₃, —CI₃, —CN, —OH, —NH₂, —COOH, substituted or unsubstituted alkyl, or substituted or unsubstituted heteroalkyl;

L² is independently a bond, —N(R¹⁰²)—, —C(O)—, substituted or unsubstituted alkylene, or substituted or unsubstituted heteroalkylene;

R¹⁰ is independently hydrogen or unsubstituted alkyl;

L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;

R¹⁰¹ is independently hydrogen, —OH, —NH₂, —COOH, —CONH₂, substituted or unsubstituted alkyl, substituted or unsubstituted aryl, or substituted or unsubstituted heteroaryl;

R¹ is:

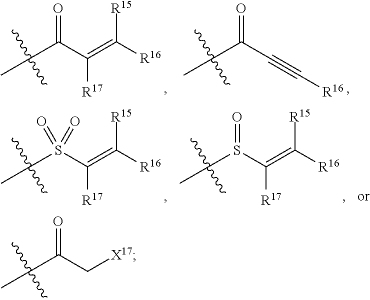

R¹⁵, R¹⁶, and R¹⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X¹⁷ is halogen;

L³ is a bond, —N(R¹⁰³)—, —O—, —S—, —C(O)—, substituted or unsubstituted alkylene, or substituted or unsubstituted heteroalkylene;

R¹⁰ is independently hydrogen, —OH, or substituted or unsubstituted alkyl; and

R³ is a target protein binding moiety.

Embodiment 9. The compound of embodiment 7 or 8, or a pharmaceutically acceptable salt thereof, wherein:

R² is independently halogen, —CF₃, unsubstituted C₁-C₃ alkyl or unsubstituted 2 to 3 membered heteroalkyl;

L² is independently a bond;

L¹ is —N(R¹⁰¹)—;

R¹⁰¹ is independently hydrogen, substituted or unsubstituted alkyl, substituted or unsubstituted aryl, or substituted or unsubstituted heteroaryl;

L³ is a bond, substituted or unsubstituted C₁-C₆ alkylene, or substituted or unsubstituted 2 to 6 membered heteroalkylene; and

R³ is a target protein binding moiety.

Embodiment 10. The compound of any one of embodiments 7 to 9, or a pharmaceutically acceptable salt thereof, wherein:

R² is independently —Cl, —Br, —F, —CF₃, —CH₃, —OCH₃, or —OCH₂CH₃;

L² is independently a bond;

L¹ is —N(R¹⁰¹)—;

R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;

R¹ is:

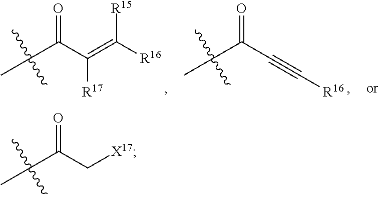

R¹⁵, R¹⁶, and R¹⁷ are independently hydrogen;

X¹⁷ is halogen;

L³ is a bond or substituted or unsubstituted 2 to 6 membered heteroalkylene; and

R³ is a target protein binding moiety.

Embodiment 11. The compound of any one of embodiments 7 to 10, or a pharmaceutically acceptable salt thereof, wherein:

L¹ is —N(R¹⁰¹)—;

R¹⁰¹ is independently hydrogen, —CH₂CH₂CN,

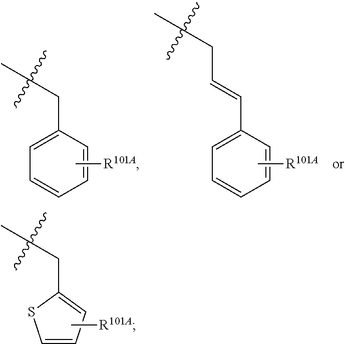

R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹ is:

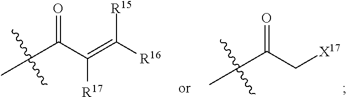

R¹⁵, R¹⁶, and R¹⁷ are independently hydrogen;

X¹⁷ is halogen;

L³ is a bond or substituted or unsubstituted 2 to 6 membered heteroalkylene; and

R³ is a target protein binding moiety.

Embodiment 12. The compound of any one of embodiments 7 to 11, or a pharmaceutically acceptable salt thereof, wherein:

L¹ is —N(R¹⁰¹)—;

R¹⁰¹ is independently hydrogen, —CH₂CH₂CN,

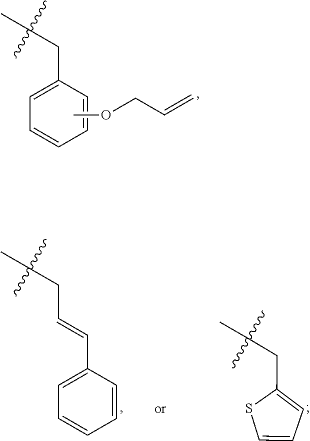

L³ is a bond or substituted or unsubstituted 2 to 6 membered heteroalkylene; and

R³ is a target protein binding moiety.

Embodiment 13. The compound of any one of embodiments 7 to 12, or a pharmaceutically acceptable salt thereof, wherein R³ is a Brd4 binding moiety.

Embodiment 14. The compound of any one of embodiments 7 to 12, or a pharmaceutically acceptable salt thereof; wherein R³ is a K-ras binding moiety.

Embodiment 15. The compound of any one of embodiments 7 to 12, or a pharmaceutically acceptable salt thereof, wherein R³ is a Bruton's tyrosine kinase (BTK) binding moiety.

Embodiment 16. The compound of any one of embodiments 7 to 12, or a pharmaceutically acceptable salt thereof, wherein R³ is an androgen receptor (AR) binding moiety.

Embodiment 17. The compound of any one of embodiments 7 to 12, or a pharmaceutically acceptable salt thereof, wherein R³ is a MYC protein binding moiety.

Embodiment 18. The compound of any one of embodiments 7 to 12, or a pharmaceutically acceptable salt thereof, wherein R³ is an N-MYC protein binding moiety.

Embodiment 19. The compound of any one of embodiments 7 to 12, or a pharmaceutically acceptable salt thereof, wherein R³ is a beta-catenin protein binding moiety.

Embodiment 20. The compound of any one of embodiments 7 to 12, or a pharmaceutically acceptable salt thereof, wherein R³ is a huntingtin (HT) protein binding moiety.

Embodiment 21. A compound selected from a group consisting of:

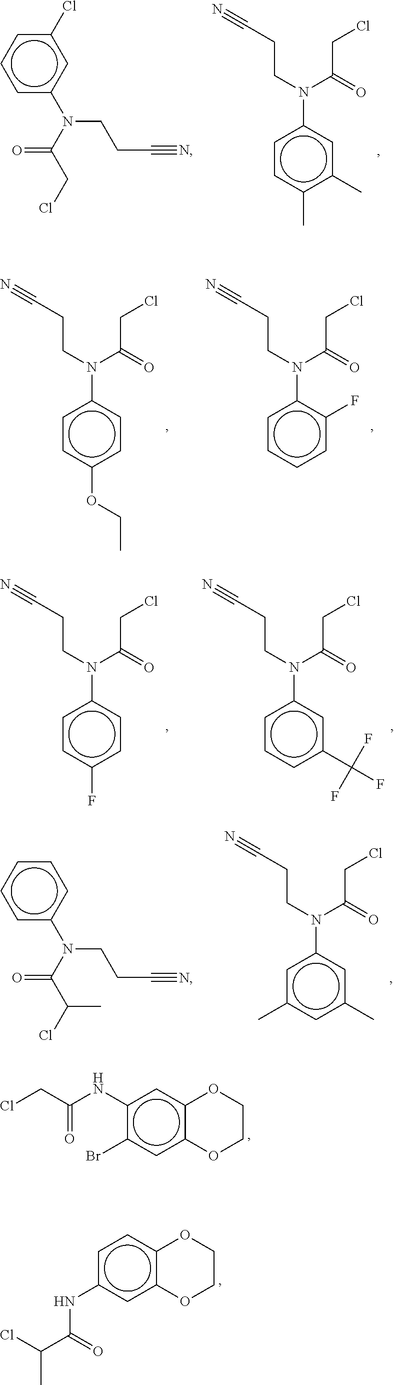

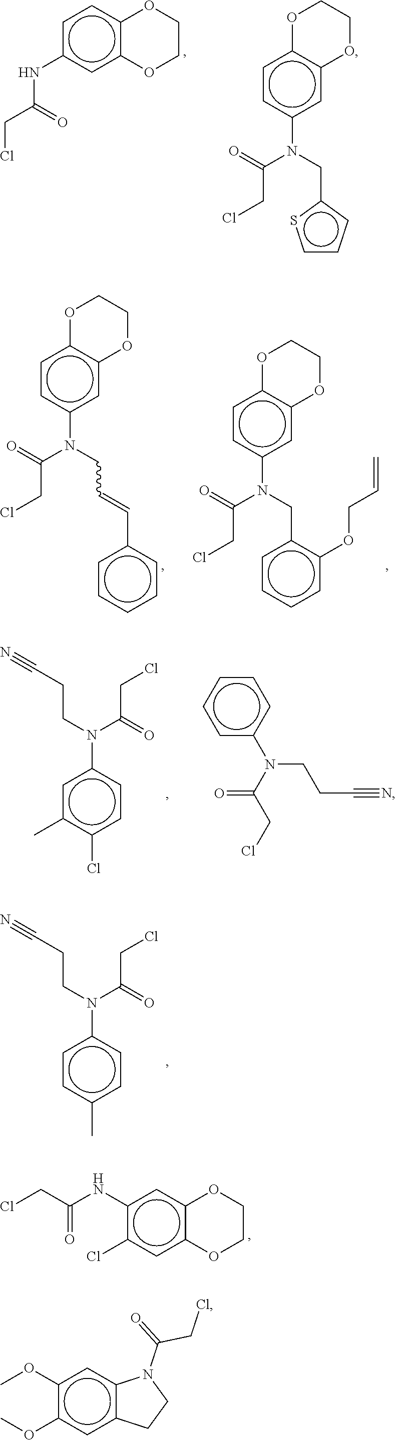

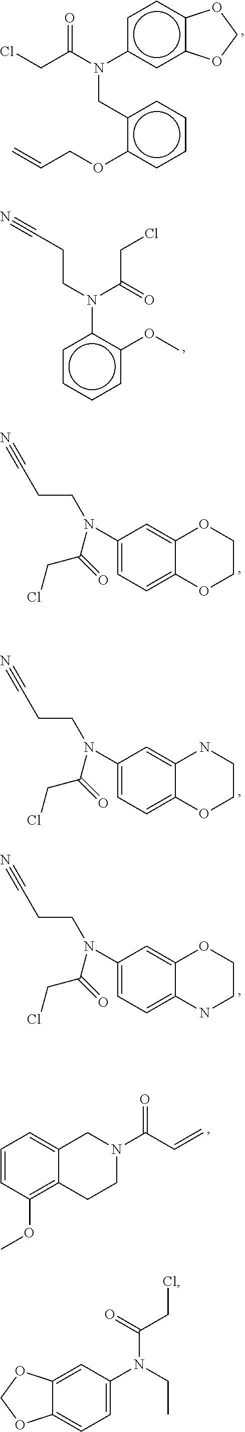

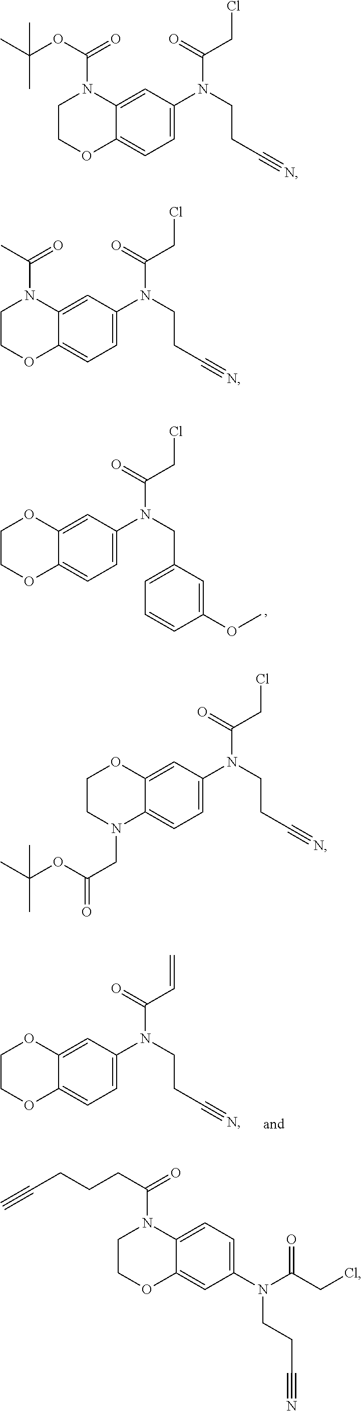

or a pharmaceutically acceptable salt thereof.

Embodiment 22. A compound having the formula:

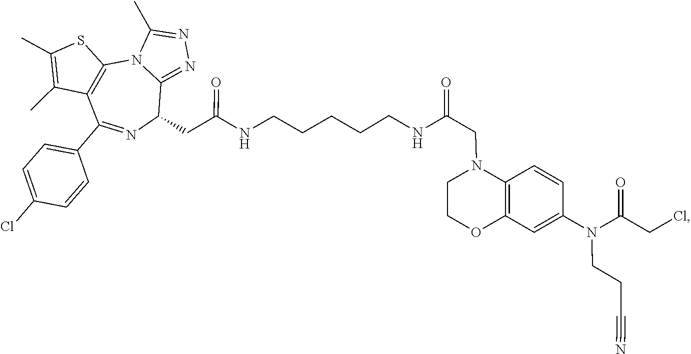 [SMILES: Cc1sc2c(c1C)C(c1ccc(Cl)cc1)=N[C@@H](CC(=O)NCCCCCNC(=O)CN1CCOc3cc(N(CCC#N)C(=O)CCl)ccc31)c1nnc(C)n1-2]

or a pharmaceutically or a acceptable salt thereof.

Embodiment 23. A pharmaceutical composition comprising the compound of any one of embodiments 1 to 22 and a pharmaceutically acceptable excipient.

Embodiment 24. A method of treating a disease in a subject in need thereof, the method comprising administering a therapeutically effective amount of FEM1B Cys 186 covalent inhibitor.

Embodiment 25. The method of embodiment 24, wherein the disease is cancer, obesity, Huntington's disease or diabetes.

Embodiment 26. The method of embodiment 25, wherein the cancer is a metastatic lung cancer, neuroblastoma, colon cancer, or renal cancer.

Embodiment 27. The method of embodiment 26, wherein the renal cancer is a KEAP1 mutated renal cancer.

Embodiment 28. The method of embodiment 25, wherein the diabetes is a type-II diabetes.

Embodiment 29. A method of treating a disease in a subject in need thereof, the method comprising administering a therapeutically effective amount of a compound having the structure: FCIM-L³-R³, wherein

FCIM is a FEM1B Cys 186 covalent inhibitor moiety,

R³ is a target protein binding moiety;

L³ is a bond, —S(O)₂—, —N(R¹⁰²)—, —O—, —S—, —C(O)—, —C(O)N(R¹⁰²)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;

R¹⁰² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 30. The method of embodiment 29, wherein the disease is cancer, neurodegenerative disease, mitochondrial disease, and diabetes.

Embodiment 31. The method of embodiment 30, wherein the cancer is a leukemia or prostate cancer.

Embodiment 32. The method of embodiment 31, wherein the prostate cancer is a castration-resistant prostate cancer.

Embodiment 33. A FEM1B protein comprising an amino acid corresponding to Cys 186, wherein said amino acid corresponding to Cys 186 is covalently bound to a (i) FEM1B Cys 186 covalent inhibitor, or (ii) a compound having the structure: FCIM-L³-R³, wherein

FCIM is a FEM1B Cys 186 covalent inhibitor moiety,

R³ is a target protein binding moiety;

L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;

R¹⁰ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and wherein the FCIM is covalently bound to said Cys 186.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: A Cellular Mechanism to Detect and Alleviate Reductive Strews

To protect their stem cell populations from damage or exhaustion, all organisms possess highly conserved and sensitive stress response pathways that detect and alleviate a wide range of adverse conditions. As one example, stem cells often reside in hypoxic niches and rely on glycolysis as their main source of energy, which limits oxidative damage to DNA, lipids, or proteins (Donato et al., 2017; Ezashi et al., 2005; Studer et al., 2000). If still too many reactive oxygen species accumulate, these cells activate the oxidative stress response to scavenge oxidizing molecules and revert oxidized proteins into their functional reduced state (Suzuki and Yamamoto, 2017). A failure to initiate the oxidative stress response can impair stem cell self-renewal and differentiation and thereby endangers tissue formation and maintenance (Tsai et al., 2013; Yamamoto et al., 2018). In a similar manner, stem cell integrity is being preserved by signaling networks that respond to protein misfolding, DNA damage, or lack of oxygen (Balchin et al., 2016; Ohh et al., 2000; Vilchez et al., 2014).

While most stresses elicit a rapid response, the underlying signaling networks also need to be turned off soon after homeostasis has been restored. In keeping with the above example, differentiating cells leave the hypoxic niche and switch to oxidative phosphorylation as a major source of ATP (Khacho et al., 2016). This metabolic shift generates the energy and building blocks needed for a change in cell fate, but it also leads to a rise in reactive oxygen species that amplify critical signaling circuits during differentiation (Holmstrom and Finkel, 2014; Rodriguez-Colman et al., 2017; Sena and Chandel, 2012). Stem cells that fail to shut off the oxidative stress response prematurely deplete oxidizing signaling molecules and cannot differentiate (Bellezza et al., 2018; Gores et al., 1989; Xiao and Loscalzo, 2019). The persistent lack of reactive oxygen species, referred to as reductive stress, ultimately results in cardiomyopathy or diabetes (Dialynas et al., 2015; Rajasekaran et al., 2007; Rajasekaran et al., 2011; Wu et al., 2016), yet how it is sensed and alleviated is unknown. As a consequence, how cells balance distinct stress responses to establish physiological levels of reactive oxygen species remains to be elucidated.

Most stress response pathways are controlled by ubiquitylation, an essential modification whose specificity is imparted by hundreds of E3 ligases (Balchin et al., 2016; Buckley et al., 2012b; Rape, 2018; Yau and Rape, 2016). As a core component of the oxidative stress response, the E3 CUL3^{KEAP1} ubiquitylates and helps degrade the transcription factor NRF2 (Wakabayashi et al., 2003). When cells experience a dangerous rise in reactive oxygen species, CUL3^{KEAP1} is inhibited and NRF2 can accumulate to drive antioxidant gene expression (Furukawa and Xiong, 2005; Zhang et al., 2004). In a similar manner, the E3 CUL2^{VHL} restricts the abundance of HIF-1α, until hypoxic stress stabilizes this transcription factor to initiate angiogenesis (Denko, 2008; Kaelin, 2007). Deletion of VHL or KEAP1 causes embryonic or early postnatal death, respectively (Gnarra et al., 1997; Wakabayashi et al., 2003), and mutation of each enzyme is a frequent cause of cancer (Cancer Genome Atlas Research, 2012; Kaelin, 2007). While these findings suggested that CUL2^{VHL} and CUL3^{KEAP1} are important for cell differentiation, how ubiquitin-dependent stress signaling is integrated into developmental programs remains incompletely understood.

Here, we searched for E3 ligases at the intersection of stress and developmental signaling to elucidate the mechanistic underpinnings of robust tissue formation and homeostasis. This led to the discovery of CUL2^{FEM1B} and its target FNIP1 as core components of the reductive stress response. Reductive stress reverts oxidation of conserved Cys residues in the degron of FNIP1 and thus allows CUL2^{FEM1B} to detect its essential substrate. The subsequent polyubiquitylation and proteasomal degradation of FNIP1 restores mitochondrial output and prevents untimely myoblast differentiation, thereby preserving both redox homeostasis and stem cell integrity. We conclude that the reductive stress response is built around a ubiquitin-dependent rheostat that tunes mitochondrial activity to cellular needs, suggesting that it is metabolic control that integrates pathways of redox stress signaling into the complex programs of metazoan development. FEM1B and FNIP1 regulate mitochondrial structure and function and are crucial metabolic regulators.

Small molecule inhibitors are the main targeted treatment towards intracellular proteins. However, small molecule inhibitors have numerous limitations. Thus, there is a need in the art for improved disease treatment options. Provided herein are solutions to these problems and other problems in the art.

Reductive stress Inhibits myoblast differentiation in vitro. To identify new regulators of stress and developmental signaling, we searched for Cullin-RING-E3 ligases (CRLs) that control myogenesis, a differentiation pathway that responds to exercise- or fasting provoked stress. As the largest class of metazoan E3s, CRLs are known to control differentiation and stress signaling, yet many of their targets remain to be discovered. We depleted each of the seven main Cullin proteins from C2C12 myoblasts and followed differentiation by immunofluorescence microscopy against myosin heavy chain (MyHC), which is expressed at later stages of myotube formation. These experiments showed that CUL2 and CUL3 were particularly important for myoblast differentiation, which is consistent with the effects of CUL3 deletion onto muscle development in mice. As depletion of other Cullins showed less dramatic effects, these E3 ligase families were not further considered in this study.

In addition to the Cullin scaffold and a RING-domain subunit that supports catalysis, CRLs contain one of ˜300 interchangeable adaptors that recruit specific targets. To comprehensively identify CUL2 or CUL3 adaptors that control myogenesis, we purified CUL2 and CUL3 from myoblasts and myotubes and determined their binding partners by mass spectrometry. We detected 19 CUL2- and 32 CUL3-adaptors, including novel candidate adaptors, such as the muscular dystrophy protein myoferlin, or factors linked to familial myopathy, such as KLHL9. We then combined this list with published adaptors, before we depleted each CUL2 and CUL3 subunit from myoblasts, induced differentiation, and recorded MyHC-positive myotubes by microscopy and automated image analysis. Critical phenotypes were confirmed with independent siRNAs to eliminate the risk of off-target effects.

Our screen revealed that the CUL3 adaptors KEAP1, BTBD9, KLHL22, and ANKFY1 were required for myotube formation, whereas depletion of the CUL2 adaptor FEM1B and the CUL3 adaptors RCBTB2 and KCTD15 had the opposite effect and improved the efficiency of this differentiation program. Loss of these adaptors barely affected nuclei count, and this did not correlate with the efficiency of myogenesis; thus, aberrant cell division or survival are unlikely account for the changes in differentiation efficiency. Notably, all adaptors that were required for myotube formation had previously been linked to disease: mutations in KEAP1 lead to lung and renal cancer; BTBD9 mutations trigger restless leg syndrome and insomnia; overexpression of KLHL22 causes breast cancer progression; and mutations in ANKFY1 result in steroid-resistant nephrotic syndrome.

We were particularly intrigued to find that depletion of the oxidative stress sensor KEAP1 prevented myoblast differentiation. While oxidative stress inhibits CUL3^{KEAP1} transiently, genetic loss of KEAP1 stabilizes the CUL3^{KEAP1} target NRF2 for prolonged periods of time and thereby elicits reductive stress. Indeed, depletion of KEAP1 in myoblasts led to prominent accumulation of NRF2 and abundant expression of its antioxidant targets. However, when we blunted persistent antioxidant signaling by co-depleting NRF2 or select NRF2 targets, differentiation was restored even though NRF2 depletion by itself did not impact myogenesis. From these findings, we infer that reductive stress caused by prolonged NRF2 accumulation impairs myogenesis in vitro. While this lent further support to the notion that reactive oxygen species fulfill critical signaling roles during differentiation, it also raised the question of how reductive stress is sensed and counteracted during normal development.

FEM1B counteracts KEAP1. We hypothesized that signaling by the still uncharacterized reductive stress response, rather than a mere absence of reactive oxygen species, prevented myogenesis during times of stress. If this were to be the case, components of the reductive stress response could be discovered as proteins whose depletion allows myotube formation to proceed in the absence of KEAP1. To identify such proteins, we designed a genetic modifier screen focused on E3 ligases as likely stress response regulators and found that loss of the CUL2 adaptor FEM1B enabled myotube formation despite lack of KEAP1. We confirmed these results by depleting KEAP1, FEM1B or both, and analyzing myoblast differentiation by microscopy and Western blotting analyses against MyHC or the earlier differentiation marker, MYOG. Intriguingly, FEM1B had already emerged from our initial screen as hit whose depletion showed the strongest increase in the efficiency of myotube formation, the opposite phenotype of loss of KEAP1.

The antagonistic relationship between FEM1B and KEAP1 was also apparent in myoblast gene expression analyses by RNAseq or qRT-PCR. As expected, depletion of KEAP1 induced NRF2 targets, such as the thioredoxin reductase TXNRD1, the glutamate-cysteine ligase subunit GCLM, NADPH dehydrogenase NQO1, or heme oxygenase HMOX1. At the same time, the loss of KEAP1 lowered the levels of muscle specific mRNAs, such as MYOG or myosin light chain MYL1, which reflects the reduced propensity of myoblasts to differentiate under these conditions. Depletion of FEM1B had the opposite effect and restricted expression of TNXRD1, GLCM, NQO1, or HMOX1, while it increased the mRNA levels of MYOG and MYL1. Importantly, concomitant loss of KEAP1 and FEM1B cancelled out each of these phenotypes of single E3 ligase depletion. These experiments therefore identified FEM1B as an antagonist of KEAP1 during myoblast differentiation. This finding raised the possibility that the E3 ligase CUL2^{FEM1B} might impact, either directly or indirectly, the reductive stress response.

CUL2^{FEM1B} targets FNIP1 for proteasomal degradation. To identify the CUL2^{FEM1B} substrate responsible for reductive stress signaling, we paid tribute to the notion that interactions between substrates and E3s are often too transient to be captured by affinity purification and therefore devised a substrate-trap by mutating Leu597 in the VHL box of FEM1B. FEM1B^{L597A} fails to integrate into CUL2 complexes and should not support ubiquitylation, it contains intact ankyrin repeats and likely retains its ability to bind substrates. As seen with other CRLs, these features were expected to prolong the association of FEM1B^{L597A} with short-lived targets and facilitate substrate identification by semi-quantitative CompPASS mass spectrometry.

Proteomic analyses confirmed that FEM1B^{L597A} was impaired in binding to CUL2 and the CRL2 subunits Elongin B and Elongin C. At the same time, FEM1B^{L597A} interacted more strongly than wildtype FEM1B with several proteins that were considered to be candidate substrates. These included the GATOR1 complex, which inhibits mTORC1 signaling during amino acid limitation, as well as the folliculin (FLCN) and FNIP1 proteins, which also bind each other. A close FNIP1 homolog, FNIP2, was not detected in these experiments. We confirmed by immunoprecipitation coupled to Western blotting that FEM1B associated with GATOR1, FLCN, and FNIP1 in a manner that was stabilized by mutation of the VHL box in FEM1B (FEM1B^{L597A}). By contrast, mutation of Cys185, a conserved residue in the ankyrin repeats of FEM1B, strongly diminished the recognition of GATOR1, FLCN, and FNIP1.

Among these candidate targets, we noted that FEM1B overexpression elicited the CUL2- and proteasome-dependent degradation of FNIP1, while deficient FEM1BC^{186S}, inactive FEM1B^{L597A} or the related CUL2 adaptor FEM1A did not have this effect. Depletion of FEM1B caused the opposite outcome and led to accumulation of endogenous FNIP1. Recombinant NEDD8-modified CUL2^{FEM1B} also efficiently polyubiquitylated FNIP1 in vitro, when incubated with the E2 enzymes UBE2D3 and UBE2R1. By contrast, expression of FEM1B did not induce degradation of FLCN, GATOR1 subunits, or FNIP2, and CUL2FEM1B did not ubiquitylate these proteins in vitro. We therefore conclude that FNIP1 is a proteolytic CUL2^{FEM1B} substrate, while FLCN or GATOR1 might interact with FEM1B indirectly or in a role that is distinct from being a degradation target.

We used a genetic approach to test whether FNIP1 degradation was critical for the stress or developmental functions of CUL2^{FEM1B}. If accumulation of FNIP1 were to be responsible for the increased differentiation upon FEM1B depletion, then cells lacking both FNIP1 and FEM1B should show the same efficiency of myotube formation as control myoblasts. This was indeed what we observed. Moreover, if FNIP1 stabilization was critical for reductive stress signaling by CUL2^{FEM1B}, we expected that loss of FNIP1 reduces myotube formation in cells lacking both FEM1B and KEAP1, which again was the case. Underscoring the specificity of these results, depletion of GATOR1 subunits, which bind but are not degraded through CUL2^{FEM1B}, did not impact differentiation in myoblasts lacking FEMIB. Together, these experiments identify FNIP1 as a crucial proteolytic CUL2^{FEM1B} substrate during reductive stress signaling.

FNIP1 is a conserved vertebrate protein that intersects with several metabolic pathways. Deletion of FNIP1 in mice results in formation of muscle fibers with overly abundant mitochondria, and FNIP1 and its binding partner FLCN are recruited to mitochondria prior to their removal by autophagy. Both FNIP1-FLCN and homologous FNIP2-FLCN complexes bind the AMP kinase that monitors cellular energy state, and they act as GTPase activating proteins during mTORC1 activation. While disease-linked variations in FNIP1 have not been found, mutations in FLCN cause Birt-Hogg-Dubé syndrome, a predisposition to renal cancer that is also results from aberrant mitochondrial activity or mutations in VHL, KEAP1 or NFE2L2.

FEM1B detects a conserved Cys degron In FNIP1. As part of a reductive stress response, we expected that degradation of FNIP1 through CUL2^{FEM1B} should be tightly regulated by the cellular redox state. Most E3 ligases detect their targets through specific substrate motifs, or degrons, that can be modulated by oxidation or other posttranslational modifications. Through systematic deletion of FNIP1 domains, we identified a stretch of ˜20 amino acids in the central region of FNIP1 that was essential for its recognition by CUL2^{FEM1B} and its proteasomal degradation.

To determine whether this FNIP1 motif comprised a transferable degron, we appended it to GFP and monitored binding of the fusion (henceforth referred to as GFP^{degron}) to CUL2^{FEM1B}. Whereas GFP was not recognized by CUL2^{FEM1B}, GFP^{degron} was readily detected by this E3 ligase. As with full-length FNIP1, GFP^{degron} showed stronger binding to FEM1B^{L597A}, which can bind but not ubiquitylate CUL2 targets, while it did not interact with the ankyrin repeat mutant FEM1B^{C186S}. We then expressed GFP^{degron} along with IRES-driven mCherry and used the ratio of GFP to mCherry as quantitative read-out for protein degradation dependent on the FNIP1 degron. Expression of FEM1B, but neither FEM1B^{L597A} nor FEM1B^{C186S}, triggered a dramatic loss of GFP^{degron}. Deletion of FEMIB by genome engineering, depletion of FEM1B by shRNAs, or proteasome inhibition provoked the opposite effect and protected GFP^{degron} from degradation. In vitro, recombinant FEM1B, but not an unrelated adaptor, bound a TAMRA-labeled degron peptide with an affinity of ˜110 nM, which is higher than what had been observed for other CUL2- or CUL5-substrates. The FNIP1 degron was also ubiquitylated by recombinant CUL2^{FEM1B}, which was dependent upon Lys residues within the peptide. Together, these experiments therefore identified a central degron that is required and sufficient for CUL2^{FEM1B} recognition and proteasomal degradation of FNIP1. Whereas this degron is conserved among FNIP1 homologs, it is not found in the otherwise closely related FNIP2 that is not recognized by CUL2^{FEM1B}.

Given the role of CUL2^{FEM1B} in reductive stress signaling, we were excited to note that the FNIP1 degron contained three invariant Cys residues, oxidation of which could potentially couple FNIP1 stability to the redox state. Importantly, Cys585 was essential for the FEM1B-dependent degradation of the GFP^{degron} reporter. Simultaneous mutation of Cys580 and Cys582 also strongly impaired the clearance of GFP^{degron} through CUL2^{FEM1B}, and mutation of all Cys residues fully protected the reporter against CUL2^{FEM1B} dependent degradation. Changing its Cys residues to Ser blocked the binding of the degron peptide to recombinant FEM1B and interfered with its ubiquitylation by CUL2^{FEM1B}. The reliance on Cys residues was even more pronounced in full-length FNIP1, where each of the three Cys as well as a neighboring His residue were required for FEM1B-binding and proteasomal degradation.

In line with this mutational analysis, treatment of the FNIP1 degron with the Cys-modifying agents iodoacetamide or N-ethylmaleimide strongly inhibited its recognition by FEM1B. NEM or iodoacetamide also blocked the CUL2^{FEM1B} dependent ubiquitylation of the degron peptide to the same extent as mutation of the Cys residues. Similar observations were made in cells, where the iodoacetamide derivative IAyne impaired the FEM1B-dependent degradation of GFP^{degron}. We conclude that CUL2^{FEM1B} relies on a degron containing unmodified Cys residues to detect its essential substrate FNIP1.

Reductive tress triggers detection of FNIP1 by CUL2^{FEM1B}. Given the reactivity of its Cys residues, we wished to monitor degron oxidation and its effects on FNIP1 detection by CUL2^{FEM1B} in cells. Unfortunately, despite treating FNIP1 immunoprecipitates with multiple proteases or even using purified peptides, we could not detect the FNIP1 degron by proteomic means. This peptide was also absent from global analyses of Cys oxidation, suggesting that it evades detection by mass spectrometry. As an alternative, we adapted a trapping assay that had been developed to monitor cellular Cys oxidation to disulfide bonds. This strategy relies on the thioredoxin TXN1, which usually reduces intracellular disulfide bonds in two steps: a Cys residue of TXN1 first attacks the disulfide bond in an oxidized protein, before a second Cys targets the mixed disulfide to release the reduced protein. A TXN1 variant lacking the second Cys, TXN1^{C35S}, fails to resolve the mixed disulfide and thus covalently traps oxidized proteins. The more a protein is trapped by TXN1^{C35S}, the more its Cys residues were oxidized to disulfide bonds. It should be noted that this approach does not capture the full extent of degron oxidation, as sulfinic or sulfonic acid derivatives are not detected.

Revealing significant degron oxidation in cells, we found that wildtype GFP^{degron}, but not a Cys-free reporter, was efficiently trapped by TXN1^{C35S}. To determine whether degron oxidation responded to changes in redox state, we increased oxidizing conditions by adding cell permeant α-ketoglutarate, which stimulates flux through the electron transport chain, or antimycin A, which escalates the load of reactive oxygen species by inhibiting mitochondrial complex III. These treatments enhanced TXN1-trapping of GFP^{degron}, indicative of increased degron oxidation. By contrast, when we imposed reductive stress to deplete reactive oxygen species, the degron was shielded from TXN1^{C35S}. Full-length endogenous FNIP1 was also trapped by TXN1^{C35S} in an antimycin A responsive manner, revealing that the FNIP1 degron is oxidized in a manner that reflects the cellular redox state.

Several observations then showed that degron oxidation modulates recognition of FNIP1 by CUL2^{FEM1B}. We noted that even a brief incubation of the degron peptide without reducing agent disrupted its binding to FEM1B. Addition of TCEP to the oxidized degron rescued its recognition by FEM1B, which documents the reversible nature of this regulatory circuit. These findings translated into cells: exposure to α-ketoglutarate or antimycin A, which increase degron oxidation, stabilized GFP^{degron}, in a manner dependent upon production of reactive oxygen species by mitochondrial complex I. By contrast, glutamine starvation or CUL3^{KEAP1} inhibition, which impose reductive stress, accelerated GFP^{degron} turnover, which required the degron Cys residues. Antimycin A also reduced binding of full-length FNIP1 to FEM1B, while reductive stress strongly promoted this interaction. In fact, if both FEM1B and FNIP1 were present at their endogenous levels, we could only detect their interaction after reductive stress had been imposed by CUL3^{KEAP1} inhibition. We conclude that reductive stress, as caused by prolonged antioxidant signaling or mitochondrial inactivity, reverts the oxidation of Cys residues in the FNIP1 degron and thereby allows CUL2^{FEM1B} to associate with his substrate. These findings identify CUL2^{FEM1B} and FNIP1 as a direct sensor to detect reductive stress.

FEM1B and FNIP1 regulate mitochondrial structure and function. CUL2^{FEM1B} and FNIP1 would constitute a bona fide reductive stress response, if degrading FNIP1 helped alleviate this adverse condition. Given the role of FNIP1-FLCN as a GAP for RagC/D, we first tested whether FNIP1 turnover impacted signaling by mTORC1, a kinase that can affect metabolism and mitochondrial function. We depleted FEM1B, FNIP1, or both, from myoblasts, starved cells to shut off mTORC1, and then added amino acids to turn on the kinase. Although depletion of FEM1B slightly improved mTORC1 activation, this was stimulated, rather than decreased, by co-depletion of FNIP1. FNIP1-FLCN also binds AMPK, which we confirmed. However, the minor increase in AMPK activity seen in cells lacking FEM1B was unaffected by co-depletion of FNIP1. FNIP1 stability therefore does not impact mTORC1 and AMPK signaling in myoblasts. Consistent with this notion, the FNIP1 homolog FNIP2 also regulates AMPK or mTORC1, yet is not targeted by CUL2^{FEM1B}.

As deletion of FNIP1 or its constitutive partner FLCN prompted an increase in oxidative muscle fibers with many mitochondria, we considered the alternative hypothesis that degradation of FNIP1 might regulate mitochondrial biogenesis or function. Strikingly, we found by transmission electron microscopy that in cells lacking FEM1B all mitochondria possessed a heavily stained, dark matrix, a condensation phenotype that had previously been ascribed to lack of substrate for oxidative phosphorylation. Consistent with this notion, many mitochondria also showed onion-like swirling of cristae, indicative of an upregulation of electron transport chain components in response to impaired oxidative phosphorylation, and some mitochondria contained large unstained blebs, as they are observed upon initiation of mitophagy after loss of the mitochondrial membrane potential. These phenotypes were quantitively rescued by co-depletion of FNIP1, which together indicated that FNIP1 stabilization impedes oxidative phosphorylation.

To directly test if FNIP1 controls mitochondrial output, we stained cells with MitoSox, which detects superoxide that is generated as a byproduct of the electron transport chain. We found that depletion of FEM1B blocked formation of mitochondrial reactive oxygen species, which was corrected by co-depletion of FNIP1. As an additional readout for mitochondrial activity, we measured the proton gradient across the mitochondrial membrane, which depends on an active electron transport chain. In line with our earlier findings, depletion of FEM1B led to an increase in mitochondria that lacked a meaningful membrane potential, which again was rectified by co-depletion of FNIP1. Depletion of FNIP1 had the opposite effect and increased the number of cells with a strong mitochondrial membrane potential. Thus, FNIP1 stabilization inhibits mitochondria, yet loss of FNIP1 can restore this organelle's function. Mitochondrial inhibition, as seen upon FEM1B depletion, reduces cellular ATP levels, which is a potent trigger of exercise-induced myogenesis. Conversely, mitochondrial reactivation upon loss of FNIP1 could trigger the production of reactive oxygen species that counteract reductive stress. The function of FNIP1 as a mitochondrial regulator could therefore explain the effects of FEM1B depletion onto both reductive stress and developmental signaling.

FEM1B and FNIP1 are crucial metabolic regulators. How does loss of FNIP1 restore mitochondrial activity? In addition to the electron transport chain, mitochondria harbor enzymes for fatty acid β-oxidation and the TCA cycle, which convert acetyl-CoA into substrate for oxidative phosphorylation and building blocks for amino acid biosynthesis. Cells fuel these reactions through small molecule-based shuttles that import pyruvate, fatty acids, or TCA cycle components into mitochondria and thereby couple cytoplasmic glucose metabolism with mitochondrial energy production.

Using a Seahorse Analyzer, we found that the glycolytic rate of myoblasts was sharply decreased in the absence of FEM1B, an effect that was partially rescued by co-depletion of FNIP1. The extracellular acidification rate, which mostly reflects conversion of pyruvate into secreted lactate, was also reduced by loss of FEM1B, and this was again partially restored by co-depletion of FNIP1. In contrast to the strong effects on glycolysis, the activity of the electron transport chain itself, monitored through the oxygen consumption rate upon addition of pyruvate, was not reduced by loss of either FEM1B or FNIP1. Stabilization of FNIP1 therefore inhibits a cytoplasmic pathway, glycolysis, which would impede flux of pyruvate into the TCA cycle and thus downregulate oxidative phosphorylation.

In line with these results, liquid chromatography coupled to mass spectrometry revealed that loss of FEM1B depleted cells of glycolytic and TCA cycle intermediates. Most of these changes were rescued by co-depletion of FNIP1, showing that they arose from stabilization of this CUL2^{FEM1B} substrate. Loss of FNIP1 by itself caused the opposite effect and increased several metabolites, such as glucose, the substrate for glycolysis. FEM1B depletion reduced cellular uptake of glucose, which was partially rescued by FNIP1 co-depletion and thus might involve an additional CUL2^{FEM1B} substrate.

In addition, many compounds that accumulated upon loss of FNIP1, yet were decreased by depletion of FEM1B, were precursors or components of mitochondrial metabolite shuttles. This included citrulline and ornithine, which replenish the TCA cycle component fumarate from cytoplasmic arginine; the fatty acid carrier acetylcarnitine and its precursor panthothenate; the NADH transporter glycerol-3-phosphate; and glutamic acid, which restores α-ketoglutarate levels. Together, these findings therefore indicated that degradation of FNIP1 increases the input into metabolism by mobilizing glucose and accelerates metabolite flux into mitochondria by increasing the availability of metabolite shuttles, both steps geared to help restore mitochondrial activity and production of reactive oxygen species in times of reductive stress.

Reductive stress, i.e. the absence of physiological reactive oxygen species, impairs disulfide bond formation in the ER and elicits the unfolded protein response, and it interferes with growth factor signaling through delayed inactivation of tyrosine phosphatases. While we show here that reductive stress also impedes myogenesis in vitro, this condition ultimately results in cardiomyopathy and diabetes. Deletion of KEAP1 in mice, however, causes lethality only after birth, and inactivation of KEAP1 is frequently observed in rapidly dividing lung and renal cancer cells. The latter findings implied that organisms possess a protective mechanism to detect and alleviate reductive stress, yet sensors or effectors of this stress response had remained unknown.

Through a genetic modifier screen in KEAP1-depleted myoblasts, we identified CUL2^{FEM1B} as a core component of the reductive stress response. Depletion of FEM1B blocked the persistent expression of NRF2 target genes and allowed myoblast differentiation to proceed if KEAP1 was absent. CUL2^{FEM1B} exerts these effects by ubiquitylating the conserved protein FNIP1 in a reaction that is tightly coupled to the cellular redox state. CUL2^{FEM1B} recognizes a Cys-rich degron in FNIP1 that is oxidized under normal conditions, yet reduced when cells experience reductive stress upon prolonged antioxidant signaling or mitochondrial inactivity. As CUL2^{FEM1B} only binds reduced, but not oxidized FNIP1, CUL2^{FEM1B} preferentially targets its key substrate during times of reductive stress. How CUL2^{FEM1B} can distinguish between reduced and oxidized Cys residues in the degron of FNIP1 will have to await structural studies of this important stress sensor.

Although FNIP1 and FLCN have been implicated in AMPK and mTORC1 signaling, we found that CUL2^{FEM1B} restricts a function of FNIP1 as a mitochondrial gatekeeper. Stabilization of FNIP1 obliterates the production of mitochondrial reactive oxygen species and induces changes in mitochondrial morphology that had been ascribed to a lack of substrate for oxidative phosphorylation. In line with these observations, accumulation of FNIP1 strongly reduced the cellular pools of glucose and of shuttle systems that import critical metabolites into mitochondria, which together will reduce the levels of FADH₂ and NADH that serve as substrate for oxidative phosphorylation. As a drop in ATP triggers myogenesis during exercise, mitochondrial inactivity could provide an explanation for the increased differentiation efficiency of cells lacking FEM1B. Moreover, the function of FNIP1 in restricting mitochondrial output might be related to the finding that FNIP1-FLCN, but not the closely related FNIP2-FLCN complex, is recruited to damaged mitochondria prior to their removal by autophagy, when aberrant organelles need to be shut down to ensure cell survival.

While stabilization of FNIP1 turned off mitochondria, loss of FNIP1, as accomplished by its degradation during reductive stress, increased the levels of glucose and mitochondrial shuttle systems. FNIP1 depletion accordingly restored mitochondrial activity in cells lacking FEM1B. Our results are reminiscent of the consequences of FNIP1 deletion in mice, which caused a switch from glycolytic to oxidative myofibers that relied on oxidative phosphorylation to produce energy. Our findings thus indicate that degradation of FNIP1 increases mitochondrial activity to produce the reactive oxygen species that counteract reductive stress, while stabilization of FNIP1 inhibits mitochondria to limit the cellular burden of reactive oxygen species. We conclude that the reductive stress response is centered on a ubiquitin-dependent rheostat that tunes mitochondrial output to the metabolic or redox needs of myoblasts.

The reductive stress response bears striking similarity to pathways controlling hypoxic and oxidative stress signaling. All three redox-protecting systems require E3 ligases of the CRL family, and in each case, substrate recognition is controlled by oxidation of substrate or E3. While prolyl hydroxylation allows ubiquitylation of VHL by the hypoxic stress E3 CUL2^{VHL}, oxidation of Cys residues in KEAP1 prevents NRF2 ubiquitylation during oxidative stress. We show that oxidation of Cys residues in FNIP1 is reversed during reductive stress and thereby triggers FNIP1 recognition by CUL2^{FNIP1}. Interestingly, while mitochondrial inactivity improved degron recognition by CUL2^{FEM1B}, the promiscuous reducing agent N-acetylcysteine did not have this effect. This suggests that FNIP1 reduction preferentially occurs in proximity to mitochondria, which emphasizes the crucial function of this organelle in detecting and alleviating reductive stress.

Loss of FNIP1's constitutive partner FLCN results in a predisposition to renal cancer. Intriguingly, the same tumor type is caused by dysregulation of the oxidative stress response by mutation of KEAP1, CUL3, or NFE2L2, persistent hypoxic stress signaling after mutation of VHL, or aberrant oxidative metabolism in the absence of known driver mutations. Kidney cells require large amounts of ATP to generate a proton gradient needed to filter toxic substances out of blood. The high metabolic needs of these cells might elicit a build-up of reactive oxygen species, prolonged antioxidant signaling, and compensatory induction of the reductive stress response. In line with this notion, all known redox stress pathways shape metabolism: HIF1α increases glycolysis in the absence of oxygen, NRF2 elicits mitochondrial biogenesis, and the reductive stress response modulates mitochondrial substrate availability for oxidative phosphorylation. Given the overlapping organismal consequences of KEAP1, FLCN, and VHL mutation and their shared ability to exert metabolic control, we propose that regulation of metabolism coordinates the stress and developmental roles of CUL2^{FEM1B}, CUL2^{VHL}, and CUL3^{KEAP1}.

While FNIP1 is a crucial CUL2^{FEM1B} substrate during reductive stress, CUL2^{FEM1B} can target more proteins. CUL2 paired with FEM1A, FEM1B, or FEM1C ubiquitylates the histone mRNA-binding protein SLBP, and all FEM1 proteins target Gli transcription factors that control important developmental transitions in C. elegans. All FEM1 proteins also act in the C-end rule pathway that eliminates proteins with carboxy-terminal degrons or truncations. As neither FEM1A nor FEM1C bind FNIP1, it is likely that FEM1B detects these targets through a distinct binding site, thereby potentially allowing cells to coordinate multiple degradation events with regulation of the redox state. We also found that CUL2^{FEM1B} binds, but does not polyubiquitylate, the GATOR1 complex that inhibits mTORC1 signaling in response to nutrient deprivation. Opposite of FNIP1 deletion, which increases oxidative muscle fibers, muscle-specific loss of GATOR1 causes a switch to glycolytic fibers.

Reductive stress inhibits myotube formation in vitro. An siRNA screen identifies KEAP1 as a crucial activator and FEM1B as an inhibitor of in vitro myoblast differentiation. C2C12 myoblasts were depleted of each substrate adaptor of CUL2 and CUL E3 ligases. Differentiation was induced in 2% horse serum, and the efficiency of myotube formation was determined by immunofluorescence analysis against the late differentiation marker myosin heavy chain (MyHC). Validation of top myogenesis screen hits by microscopy. C2C12 myoblasts were depleted of KEAP1 or FEM1B and myotube formation was determined by immunofluorescence microscopy against MyHC. Quantification of myogenesis efficiency was conducted for two independent siRNAs each against KEAP1 or FEM1B. Each experiment included three biological replicates. Validation of top myogenesis screen hits by Western blotting. C2C12 myoblasts were depleted of KEAP1 or FEM1B and expression of the myogenesis markers MYOG and MyHC was determined at the indicated times of differentiation by Western blotting using specific antibodies. Myogenesis can be rescued in the absence of KEAP1 by co-depletion of NRF2. C2C12 cells were depleted of KEAP1, NRF2, or both, and differentiation was induced as described above. The differentiation efficiency was monitored by Western blotting against MYOG and MyHC, using specific antibodies. Myogenesis can be rescued in the absence of KEAP1 by co-depletion of NRF2 or antioxidant NRF2 target genes. C2C12 myoblasts were depleted of KEAP1, NRF2, select antioxidant target of NRF2 (GSR, GCLC), or combinations thereof. Differentiation was induced, and the efficiency of myotube formation was determined by immunofluorescence microscopy against MyHC.

The CUL2 adaptor FEM1B counteracts KEAP1 during myoblast differentiation. A genetic modifier screen identifies FEM1B as a KEAP1 antagonist. C2C12 myoblasts were treated with siRNAs against KEAP1 and each of the known CUL2 substrate adaptors. Three days after initiation of differentiation, myotube formation was determined by immunofluorescence microscopy against MyHC. KEAP1 and FEM1B antagonize each other during in vitro myogenesis. C2C12 cells were depleted of KEAP1, FEM1B, or both, and induced to differentiate. Myotube formation was monitored by immunofluorescence microscopy against MyHC. FEM1B antagonizes KEAP1 during myogenesis. C2C12 cells were depleted of KEAP1, FEM1B, or both, induced to differentiate, and analyzed by Western blotting against NRF2, MYOG, or MyHC. The antagonism between KEAP1 and FEM1B is detected by gene expression analysis. C2C12 myoblasts were depleted of KEAP1, FEM1B, or both. mRNA abundance was measured by RNAseq and analyzed by unsupervised clustering. Validation of RNAseq analysis by qRT-PCR of select NRF2 target genes in C2C12 myoblasts depleted of KEAP1, FEM1B, or both.

FNIP1 is a critical CUL2^{FEM1B} substrate during myogenesis and reductive stress signaling. GATOR1, FLCN, and FNIP1 are candidate CUL2^{FEM1B} substrates. Wildtype ^{FLAG}FEM1B or ^{FLAG}FEM1B^{L597A} were affinity-purified from C2C12 myoblasts and binding partners were determined by CompPASS mass spectrometry. Total spectral counts (TSC) were normalized to input levels. Analysis of FEM1B binding of GATOR1, FLCN, and FNIP1 by affinity-purification and Western blotting. Wildtype ^{FLAG}FEM1B, ^{FLAG}FEM1B^{C186S} (a mutation of a conserved residue in the substrate-binding ankyrin repeats of FEM1B), or ^{FLAG}FEM1B^{L597A} were affinity-purified from C2C12 myoblasts that also expressed the indicated HA-tagged proteins. Co-purifying HA-tagged proteins were determined by Western blotting. Overexpression of FEM1B elicits proteasome-dependent degradation of FNIP1. C2C12 cells were co-transfected with ^{HA}FNIP1 and FEM1B, FEM1B^{L597A}, or FEM1A. Proteasome inhibitor MG132 was added, and FNIP1 levels were determined by Western blotting. Depletion of FEM1B by two independent siRNAs results in accumulation of endogenous FNIP1, as determined by Western blotting using specific antibodies. CUL2^{FEM1B} polyubiquitylates FNIP1 in vitro. FNIP1-FLCN complexes were purified from 293T cells and incubated with recombinant CUL2^{FEM1B} (unmodified or Nedd8-modified), E1, ubiquitin, and the E2 enzymes UBE2D3 and/or UBE2R1. Reactions were analyzed by gel electrophoresis and Western blotting against FNIP1 or FLCN. FNIP1 is a physiologically important CUL2^{FEM1B}-substrate. C2C12 myoblasts were depleted of FEM1B, FNIP1, or both, differentiation was initiated, and the efficiency of myotube formation was analyzed by immunofluorescence microscopy against MyHC. FNIP1 stabilization restores differentiation during reductive stress. C2C12 myoblasts were depleted of KEAP1, FEM1B, FNIP1, or combinations thereof. Differentiation was induced, and the efficiency of myotube formation was determined by immunofluorescence analysis against MyHC.

FEM1B binds a conserved central degron in FNIP1. Deletion analysis of FNIP1 identifies a stretch of 20 amino acids that is required for its binding to FEM1B and its proteasomal degradation. FNIP1 variants were expressed in cells and tested for interaction with ^{FLAG}FEM1B by co-immunoprecipitation, and for degradation by measuring their abundance in the presence or absence of FEM1B. The FNIP1 degron is required for FEM1B binding and proteasomal degradation. Wildtype FNIP1 or a variant lacking its candidate degron (FNIP1^{Δ561-591}) were co-expressed with FLCN and ^{FLAG}FEM1B or inactive ^{FLAG}FEM1B^{L597A}. FEM1B variants were immunoprecipitated and bound FNIP1 and FLCN were detected by Western blotting. The FNIP1 degron is sufficient to mediate FEM1B-dependent degradation. The FNIP1 degron was fused to GFP (GFP^{degron}) and expressed from the same plasmid as an IRES controlled mCherry. mCherry provides normalization for GFP fluorescence in each cell. Cells were transfected with FEM1B, catalytically inactive FEM1B^{L597A}, or substrate-binding deficient FEM1B^{C186S}. Levels of GFP^{degron} and mCherry were determined by FACS, and their ratio is depicted for presence or absence of FEM1B variants. Deletion of FEM1B prevents FNIP1-degron dependent degradation. The FEM1B locus was deleted in 293T cells using CRISPR/Cas9. Cells were transfected with the GFP^{degron}/mCherry reporter, and analyzed for GFP and mCherry fluorescence by FACS. FEM1B directly binds the FNIP1 degron in vitro. A TAMRA-labeled FNIP1 degron peptide was incubated with purified FEMIB or the control CRL1 adaptor FBXL17, and binding was measured by fluorescence polarization of the peptide. The FNIP1 degron is sufficient to mediate CUL2^{FEM1B}-dependent ubiquitylation. The TAMRA-labeled FNIP1 degron was incubated with recombinant CUL2^{FEM1B} (unmodified or NEDD8-modified), E1, ubiquitin, ATP and the E2 enzymes UBE2D3 and/or UBE2R1. Ubiquitylation was monitored by gel electrophoresis and fluorescence detection.

Reactive Cys residues are essential for FNIP1 degron function. Mutation of Cys585 or Cys580/582 of the FNIP1 degron strongly impedes degron function. GFP-fusions between a wildtype FNIP1 degron or variants lacking the indicated Cys residues were co-expressed with IRES-driven mCherry and the GFP/mCherry ratio was determined by FACS. Cells also expressed FEM1B or not. Cys residues are required for degron recognition by FEM1B in vitro. TAMRA-labeled degron peptides, including wildtype degron, a Cys-free variant, or a Lys-free variant, were incubated with recombinant FEM1B. Degron binding was monitored through fluorescence polarization. Cys residues are essential for degron ubiquitylation by CUL2^{FEM1B}. TAMRA-labeled FNIP1 degron peptides (wildtype, a degron lacking Cys residues, and a degron lacking Lys residues) were incubated with recombinant Nedd8-modified CUL2^{FEM1B}, ubiquitin, ATP, E1, and UBE2D3/UBE1R1 as E2 enzymes. Ubiquitylation reactions were followed by gel electrophoresis and fluorescence imaging. Each degron Cys residue as well as a neighboring His residue are required for CUL2^{FEM1B} binding and proteasomal degradation of full-length FNIP1. HA-tagged FNIP1 variants were co-expressed with either wild-type ^{FLAG}FEM1B or inactive ^{FLAG}FEM1B^{L597A}. Levels of indicated FNIP1 variants in the presence or absence of FEM1B were monitored by gel electrophoresis and Western blotting. E3 ligase recognition of FNIP1 variants was determined by α-FLAG affinity-purification of FEM1B followed by Western blotting. Treatment of the degron peptide with NEM prevents binding to FEM1B. The TAMRA-labeled FNIP1 degron peptide was incubated in either buffer or NEM, reactions were quenched with DTT, and binding to FEM1B was monitored by fluorescence polarization. Degron modification with Cys-reactive NEM or iodoacetamide prevents polyubiquitylation of the FNIP1 degron peptide. The TAMRA-labeled FNIP1 degron peptide was incubated in buffer, NEM, or iodoacetamide. Reactions were quenched, and the degron was added to recombinant Nedd8-modified CUL2^{FEM1B}, ubiquitin, ATP, E1, and UBE2D3/UBE2R1. Ubiquitylation reactions were followed by gel electrophoresis and fluorescence imaging (to monitor degron ubiquitylation) or Coomassie staining (to monitor substrate independent CUL2^{FEM1B} activity). The Cys-reactive, cell-permeable IAyne stabilizes GFP^{degron} in cells, as seen by a right shift of the GFP/mCherry ratio observed by FACS.

The FNIP1 degron is redox sensitive. The FNIP1 degron is oxidized in cells. A wildtype FNIP1 degron or a degron variant lacking all Cys residues were fused to GFP and tested for trapping by TXN1^{C35S}. Trapping was determined by TXN1^{C35S} affinity-purification, resolution of mixed disulfide bonds in reducing buffer, and gel electrophoresis and Western blotting against HA-tagged GFP^{degron}. Degron oxidization is increased by mitochondrial production of reactive oxygen species. Cells were treated with cell-permeable α-ketoglutarate or antimycin A, both of which increase the cellular load with reactive oxygen species. TXN1^{C35S}-trapping of the degron peptide was determined as described above. The degron is reduced by prolonged antioxidant signaling or mitochondrial inactivity. Cells were either depleted of glutamine, which limits substrate for the TCA cycle and, as a consequence, inhibits oxidative phosphorylation. Alternatively, cells were exposed to bardoxolone, a KEAP1 inhibitor that stabilizes antioxidant NRF2. TXN1^{C35S}-trapping of the degron peptide was determined as described above. Degron recognition by FEM1B is regulated by reversible oxidation. The TAMRA-labeled FNIP1 degron was dissolved in a buffer that either contained (TCEP) or lacked (ox) the reducing agent TCEP. After binding to recombinant FEM1B had been measured by fluorescence polarization, the oxidized degron was treated with TCEP and binding to FEM1B was monitored again (ox→TCEP). FNIP1 degron-dependent degradation is prevented by mitochondrial production of reactive oxygen species. Cells expressing GFP^{degron} and mCherry were treated either with cell-permeable α-ketoglutarate or antimycin A, and the GFP/mCherry ratio was determined by FACS. FNIP1 degron-dependent reporter degradation is accelerated by persistent absence of reactive oxygen species, i.e. reductive stress. Cells expressing GFP^{degron} and mCherry were either depleted of glutamine or treated with the KEAP1 inhibitor bardoxolone, and the GFP/mCherry ratio was determined by FACS. The interaction between FNIP1 and FEM1B is sensitive to changes in the cellular redox state. Cells were treated with α-ketoglutarate, antimycin A, oligomycin, or the KEAP inhibitor TBHQ. ^{FLAG}FEM1B was affinity-purified, and binding to HA-tagged FNIP1, FLCN, or GATOR1 subunits was determined by gel electrophoresis and Western blotting. Endogenous FEM1B and FNIP1 preferentially interact during reductive stress. The FEM1B locus of 293T cells was fused to a FLAG epitope by CRISPR/Cas9-dependent genome editing. Cells that also expressed a dominant-negative variant of CUL2 to stabilize CRL2-substrate interactions were then treated with oligomycin (OM), antimycin A (AM) or TBHQ. Endogenous FEM1B was affinity-purified and bound endogenous FNIP1 was detected by gel electrophoresis and Western blotting.

FNIP1 levels control mitochondrial metabolism. Transmission electron microscopy (TEM) reveals changes in mitochondrial structure brought about by depletion of FEM1B. C1C12 myoblasts were depleted of FEM1B, FNIP1, KEAP1, or combinations thereof and processed for TEM analysis. In the absence of FEM1B, mitochondria showed dramatic changes to matrix density and membrane organization, which was completely rescued by co-depletion of FNIP1. FNIP1 stabilization shuts off production of mitochondrial reactive oxygen species. C2C12 myoblasts were depleted of FEM1B, FNIP1, KEAP1, or combinations thereof and stained for mitochondrial superoxide using MitoSox. Images were analyzed by automated immunofluorescence microscopy. Depletion of FEM1B reduces the mitochondrial membrane potential. C2C12 myoblasts were depleted of FEM1B, FNIP1, KEAP1, or combinations thereof, incubated with the mitochondrial membrane potential dye TMRE, and analyzed by FACS. CCCP-treated cells were used as control. FEM1B depletion inhibits glycolysis in a FNIP1-dependent manner. C2C12 myoblasts were depleted of FEM1B, FNIP1, KEAP1, or combinations thereof and the extracellular acidification rate was determined using a Seahorse Analyzer. FEM1B and FNIP1 control cellular metabolism. C2C12 myoblasts were depleted of FEM1B, FNIP1, KEAP1, or combinations thereof and the abundance of polar metabolites was determined by liquid chromatography and mass spectrometry. Reductive stress reverts the oxidation of critical Cys residues in the FNIP1 degron, leading to recognition of FNIP1 by CUL2^{FEM1B}, polyubiquitylation, and proteasomal degradation. The loss of FNIP1 accelerates metabolite import into mitochondria and thus triggers production of reactive oxygen species to counteract reductive stress.

CRL2 and CRL3 E3 ligases are required for myoblast differentiation. CUL2 and CUL3 are required for myotube differentiation in vitro. C2C12 myoblasts were depleted of CUL1, CUL2, CUL3, CUL4a, CUL4a, CUL5, and CUL7, differentiation was induced, and formation of myotubes was followed by immunofluorescence microscopy against MyHC and automated image analysis. CRL adaptors affect myotube formation independently of effects on cell division or survival. C2C12 myoblasts were depleted of the indicated CRL2- or CRL3-adaptors and subjected to differentiation. Nuclei were stained by Hoechst and counted by automated image analysis. No correlation between effects on differentiation and nuclei count was observed.

FNIP1 is a specific substrate of CUL2^{FEM1B}. FEM1B^{L597A} does not efficiently bind CUL2 in the assay tested. ^{FLAG}FEM1B or ^{FLAG}FEM1B^{L597A} were expressed in 293T cells and affinity-purified on αFLAG agarose. Bound CUL2 was detected by gel electrophoresis and Western blotting using specific antibodies. FEM1B^{L597A} shows enhanced binding to FNIP1, FLCN, and GATOR1 subunits. ^{FLAG}FEM1B or ^{FLAG}FEM1B^{L597A} were affinity-purified from 293T cells that also expressed ^{HA}FNIP1, ^{HA}FLCN, or HA-tagged GATOR1 subunits. Binding of HA-tagged proteins to FEM1B was detected after αFLAG affinity-purification by gel electrophoresis and Western blotting using αHA antibodies. CUL2 activity is required for FEM1B-dependent degradation of FNIP1 in the assay tested. Cells were transfected with ^{FLAG}FEM1B, ^{HA}FNIP1, ^{HA}FLCN, and dominant-negative ^{HA}CUL2, as indicated, and FNIP1 abundance was determined by gel electrophoresis and Western blotting. FEM1B does not induce the degradation of FNIP2, a close homolog of FNIP1. Cells were transfected with ^{HA}FNIP2 and the indicated FEM1B constructs, and FNIP2 levels were determined by gel electrophoresis and Western blotting using αHA antibodies. CUL2^{FEM1B} does not ubiquitylate GATOR1 subunits in the assay tested. NRPL2 and NRPL3 were purified from insect cells, incubated with recombinant CUL2 or CUL2^{FEM1B}, ATP, E1, UBE2D3 and UBE2R1 as E2s, and ubiquitin, and analyzed for ubiquitylation by gel electrophoresis and Western blotting. Co-depletion of NRPL2, NRPL3, or DEPDC5 does not rescue the increased myogenesis observed in cells lacking FEM1B. C2C12 myoblasts were transfected with the indicated siRNAs, induced to differentiate, and analyzed by immunofluorescence microscopy against MyHC.

FNIP1 contains a conserved degron for recognition by CUL2^{FEM1B}. The FNIP1 degron is sufficient to mediate binding to FEM1B. 293T cells were co-transfected with ^{FLAG}FEM1B or ^{FLAG}FEM1B^{L597A} and GFP or GFP^{degron} (GFP-FNIP1⁵⁶²⁻⁵⁹¹). ^{FLAG}FEM1B variants were affinity-purified and bound GFP was detected after gel electrophoresis and Western blotting. Degron binding to FEM1B is specific. ^{FLAG}FEM1B or the substrate-binding deficient ^{FLAG}FEM1B^{C186S} were co-transfected with GFP^{degron}. ^{FLAG}FEM1B variants were affinity-purified, and bound GFP was detected by gel electrophoresis and Western blotting. Depletion of FEM1B by four independent shRNAs stabilizes the GFP-degron fusion, as observed after co-expression of GFP^{degron} and mCherry and analysis by FACS. Proteasome inhibition with carfilzomib stabilizes the GFP^{degron} reporter, as determined by FACS. The FNIP1 degron is not present in FNIP2, a protein otherwise highly similar to FNIP1. FNIP2 is also not recognized by CUL2^{FEM1B}. F. The Cys residues of the FNIP1 degron are invariant among vertebrate FNIP1 homologs.

Cysteine residues are essential for FNIP1 degron function. Cysteine residues in the FNIP1 degron are essential for FEM1B-dependent degradation. GFP fused to the wildtype degron (WT) or a degron with all three Cys residues mutated to Ser (CS) was co-expressed with FEM1B as indicated. The GFP vector contained mCherry translated from an IRES sequence for normalization. The GFP/mCherry ratio was measured as an indication for GFP stability by FACS. Cys residues are essential for FNIP1 degron function, as seen by Western blotting. The indicated HA-tagged FNIP1 variants were co-expressed with FLCN and FLAG-tagged FEM1B or FEM1B^{L597A}. FEM1B variants were immunoprecipitated and co-purifying FNIP1 was detected by gel electrophoresis and αHA-Western.

FEM1B recognizes an unmodified FNIP1 degron. Modification of degron Cys residues prevents binding to FEM1B. A TAMRA-labeled FNIP1 degron peptide was incubated with buffer or NEM. Binding to recombinant FEM1B was monitored by fluorescence polarization. Reversible oxidation controls degron recognition by FEM1B. Extracts of cells expressing an HA-tagged FNIP1 fragment containing the degron (FNIP1⁵⁶⁷⁻⁸⁹³) were incubated with recombinant ^{MBP}FEM1B. Reducing agent (TCEP) or a Cys-reactive modifier (iodoacetamide) were added. ^{MBP}FEM1B was purified on maltose agarose and bound FNIP1 was detected by gel electrophoresis and αHA-Western blotting. The FNIP1 degron is oxidized in cells in the context of full-length FNIP1. ^{FLAG}TXN1^{C35S} was affinity-purified from cells that have been treated with antimycin A as indicated, and co-eluting endogenous FNIP1 and FLCN were detected by Western blotting. Stabilization of the FNIP1 degron reporter in antimycin A treated cells is dependent on the activity of the electron transfer chain. Stability of GFP^{degron} was monitored by FACS, as described before. Cells were either treated with antimycin A, rotenone (which inhibits electron delivery to complex III), or both. Reductive stress-dependent degradation of the FNIP1 degron reporter is dependent on the Cys residues of the degron. GFP fused to the wildtype degron (WT) or a degron with all three Cys residues mutated to Ser (CS) was co-expressed with FEM1B, and cells were depleted off glutamine to induce reductive stress, as indicated. mCherry was translated from an IRES sequence for normalization. The GFP/mCherry ratio was measured as an indication for GFP stability by FACS.

FEM1B and FNIP1 are central regulators of metabolism. FEM1B does not strongly affect mTORC1 signaling in myoblasts in the assay tested. C2C12 myoblasts were depleted of FEM1B, FNIP1, or combinations thereof. Cells were starved, before amino acids were added to rapidly turn on mTORC1. mTORC1 activity was monitored by measuring levels of phosphorylated S6 kinase by gel electrophoresis and Western blotting. FNIP1 binds AMPK in myoblasts. ^{FLAG}FNIP1 was expressed in C2C12 myoblasts, and binding partners were determined by affinity-purification and mass spectrometry. FEM1B does not strongly affect AMPK signaling in the assay tested. C2C12 myoblasts were depleted of FEM1B, FNIP1, or combinations thereof, and AMPK signaling was monitored by measuring phosphorylated ACC and phosphorylated AMPK by gel electrophoresis and Western blotting. Depletion of FEM1B reduces the extracellular acidification rate, which is partially rescued by co-depletion of FNIP1. Depletion of FEM1B or FNIP1 do not affect the activity of the electron transport chain, if pyruvate is added to cells. The oxygen consumption rate (OCR) was measured for C2C12 cells transfected with the indicated siRNAs using a Seahorse Analyzer. FEM1B depletion inhibits glucose uptake. FEM1B depletion does not inhibit the mitochondrial electron transfer chain per se. N-acetylcysteine (NAC) does not affect the stability of the GFP^{degron} reporter, as measured by FACS.

FNIP1 and FEM1B control the abundance of mitochondrial metabolites and shuttles. C2C12 cells were transfected with siRNAs targeting FNIP1, FEM1B, or both. Polar metabolites were extracted and analyzed by liquid chromatography and mass spectrometry.

Materials and Methods

NGS Library Prep and RNA-seq. Total RNA was extracted from sub-confluent C2C12s treated with siFem1b, siKeap1, siFem1b-SiKeap1, or siControl siRNAs (in triplicate) using a NucleoSpin Plus RNA extraction kit (Machery-Nagel). NGS libraries were made using a TruSeq Stranded Total RNA kit (Ilumina), with an average size of 250 bp. Libraries were prepared by the UC Berkeley Functional Genomics Laboratory. Paired-end RNA-sequencing was done using a HiSeq400 (Illumina). Sequencing of the libraries was done two times to obtain technical replicates.

RNA-seq Alignment, Expression Analysis and Transcription Factor Enrichment. We used the Kallisto-Sleuth pipeline to perform differential gene expression analysis between samples. Briefly, paired-end RNA-seq reads were aligned using Kallisto, using the mm10 Mus musculus reference transcriptome and 200 bootstrap steps. For differential expression analysis, the R Sleuth package was used. To obtain log 2 fold changes, we had to implement the following transformation function during the initial sleuth object (so) preparation step:

so→sleuth_prep(s2c,˜condition/bio_samp,extra_bootstrap_summay=TRUE,target_mapping=t2g,transformation_function=function(x)log 2(x+0.5))

To identify significant differentially expressed genes, the following conditions were compared: siControl v siFem1b; siControl v siKeap1; siFem1b v siKeap1. From each comparison, significant differentially expressed genes with a qval≤0.075 were kept. This generated four different gene lists, which were then merged together. This gene list was used to generate heat maps for data visualization. Heat maps of significant differentially expressed genes were generated using the R “heatmap.2” package, normalized by row, and using unsupervised clustering applying the “ward.D2” option. Transcription factor enrichment for significant differentially expressed genes was done using the ChEA3 and oPOSSUM algorithms.

Transmission electron microscopy. siControl, siFem1b, siFnip1, and siFnip1-siFem1b treated C2C12 cells were grown to 50% confluence and fixed in 2% guleteraldehyde: 0.1 M sodium cacodylate (pH 7.2) for 20 min, followed by 3 washes with 0.1 M sodium cacodylate buffer. Cells were then gently harvested and collected in 1.5 mL Eppendorf tubes, followed by imbedding in agarose plugs. After solidifying, agar plugs containing the specimens were carefully cut into ˜2.5 mm³ slices. Slices were stained in 1% osmium tetroxide in 0.1 M sodium cacodylate for 1 hr, followed by 1.38% potassium ferricyanide for 1 hr. Stained samples were step-dehydrated in acetone (35%, 50%, 70%, 80%, 95%, 100%, 100%) for 10 min at each step. Dehydrated samples were then step-infiltrated with acetone:Epon resin (2:1, 1:1, 1:2 for 1 hr each). After final acetone:resin infiltration, samples were embedded in pure Epon resin at room temperature, overnight, followed by curing at 65° C. for two days. Cured samples were then sliced using a Leica UC 6 microtome, taking 70 nm sections. Sliced sections were picked up on 100 mesh formvar-coated copper grids, then stained with 2% aqueous uranyl acetate for 5 min, followed by 2% lead citrate for 2 min. Grids were examined under a Tecnai 12 TEM at 120 kV.

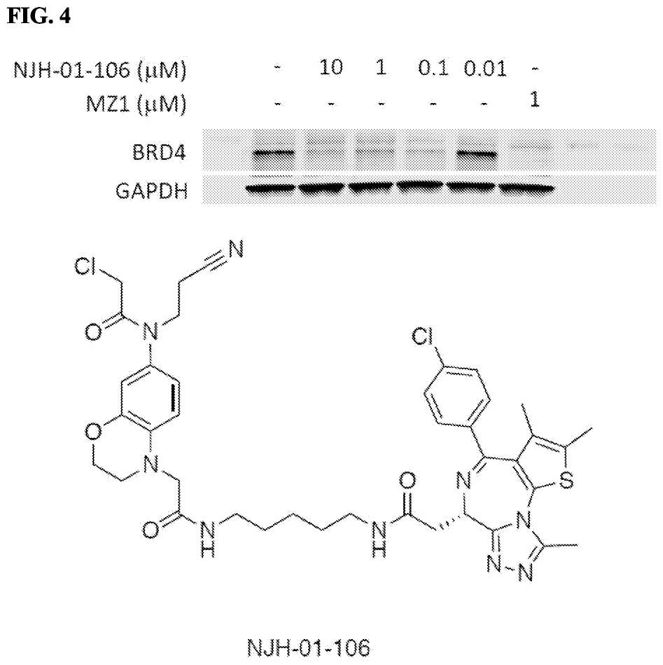

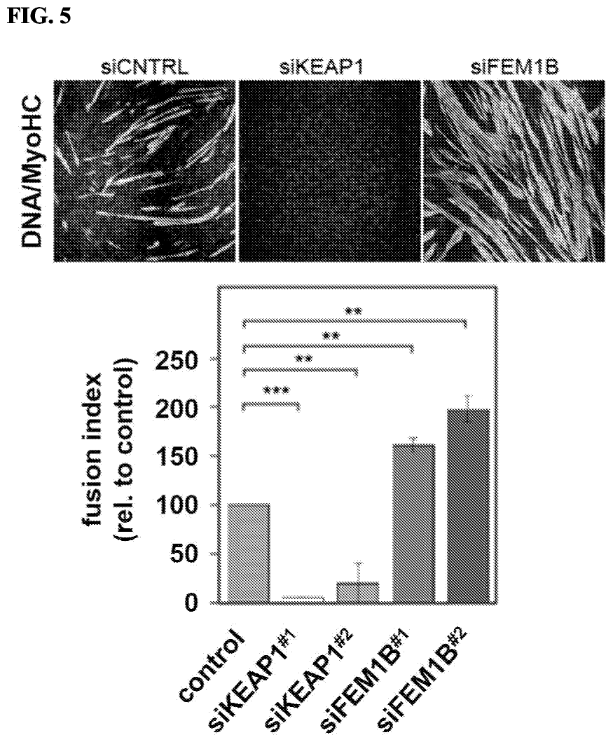

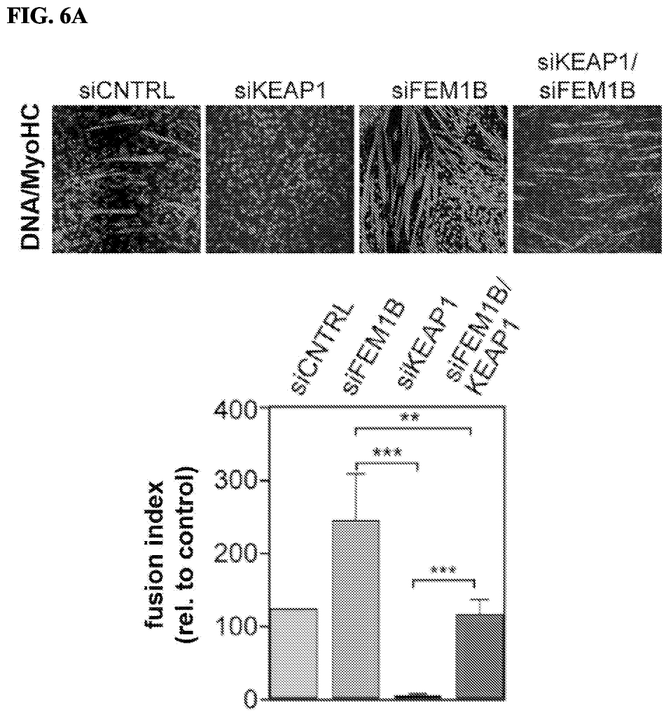

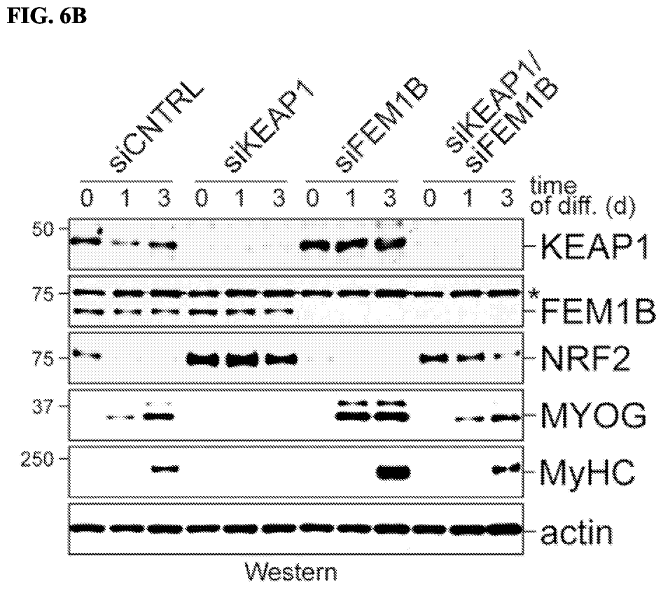

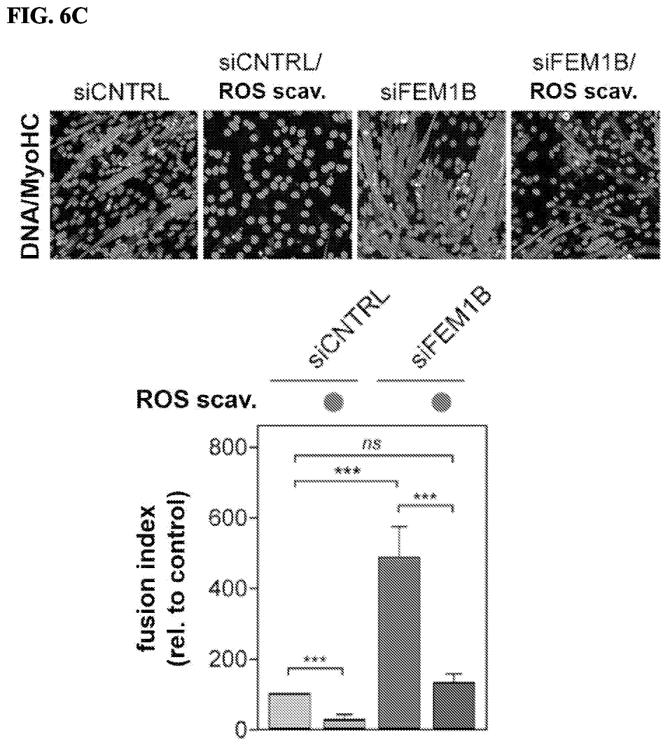

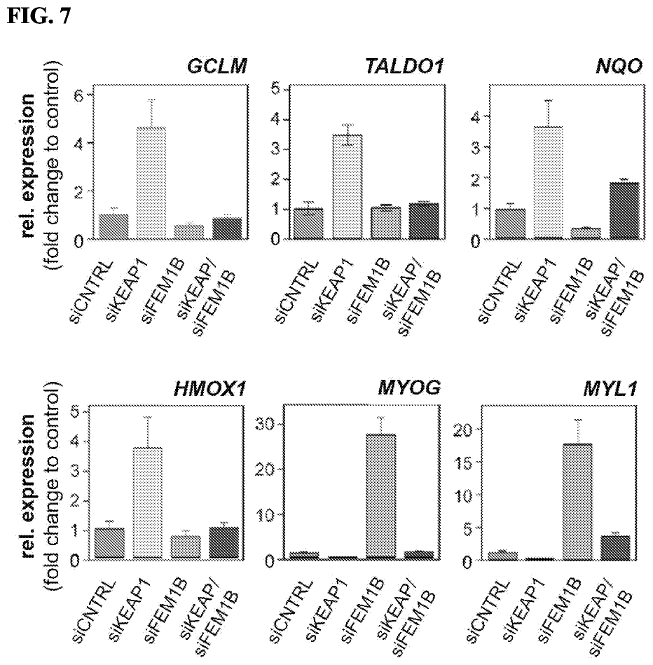

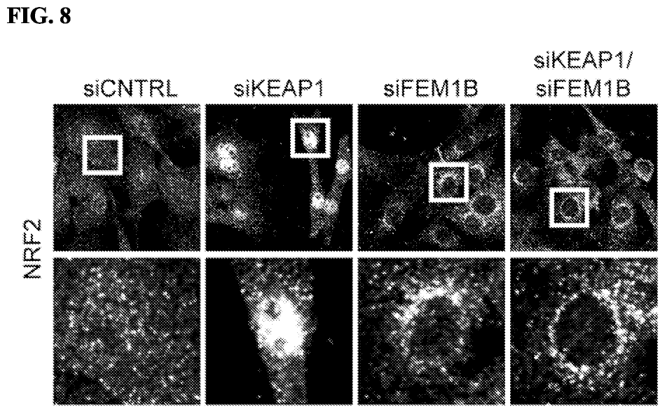

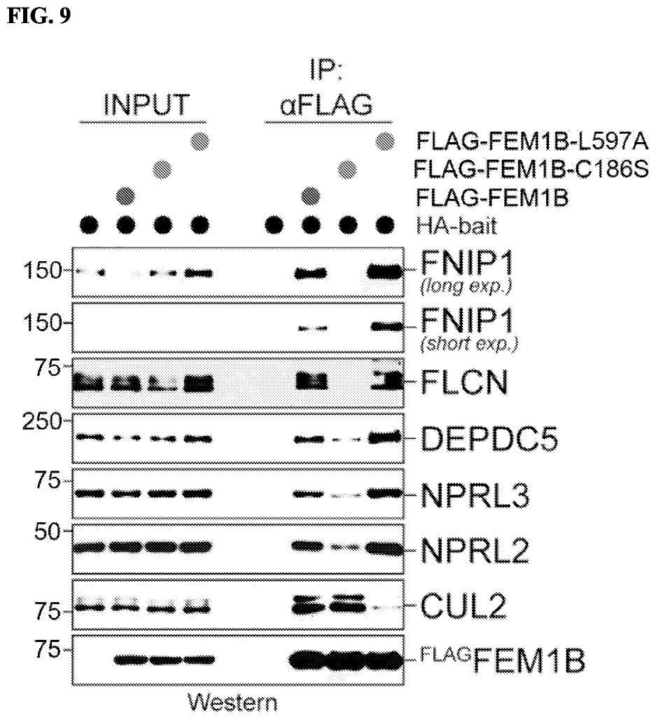

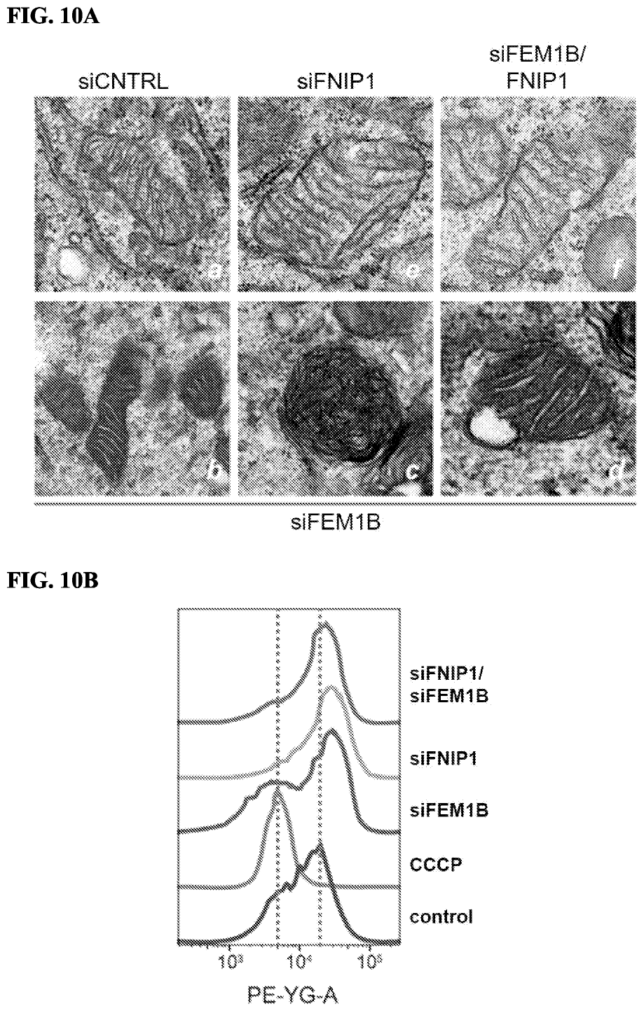

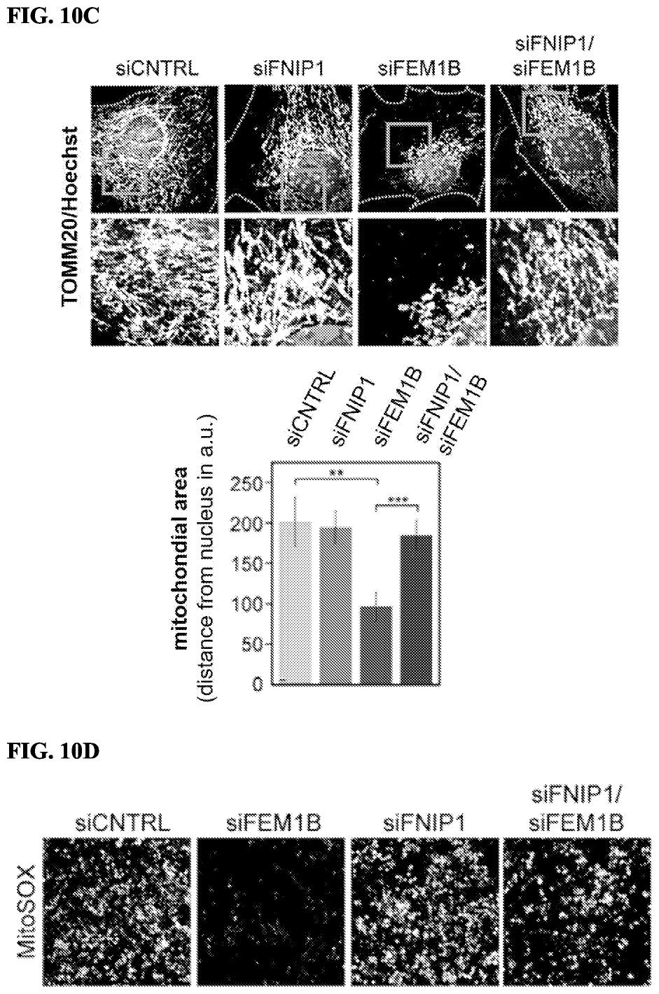

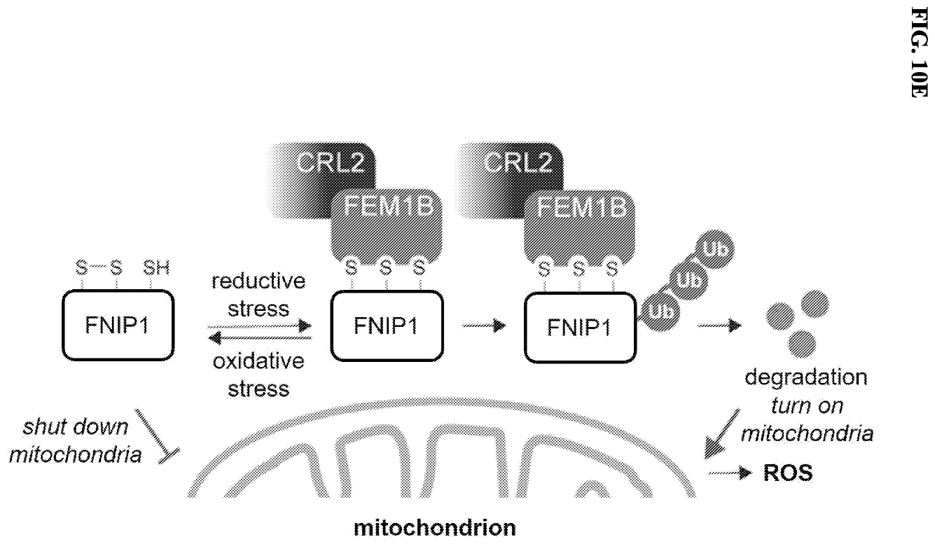

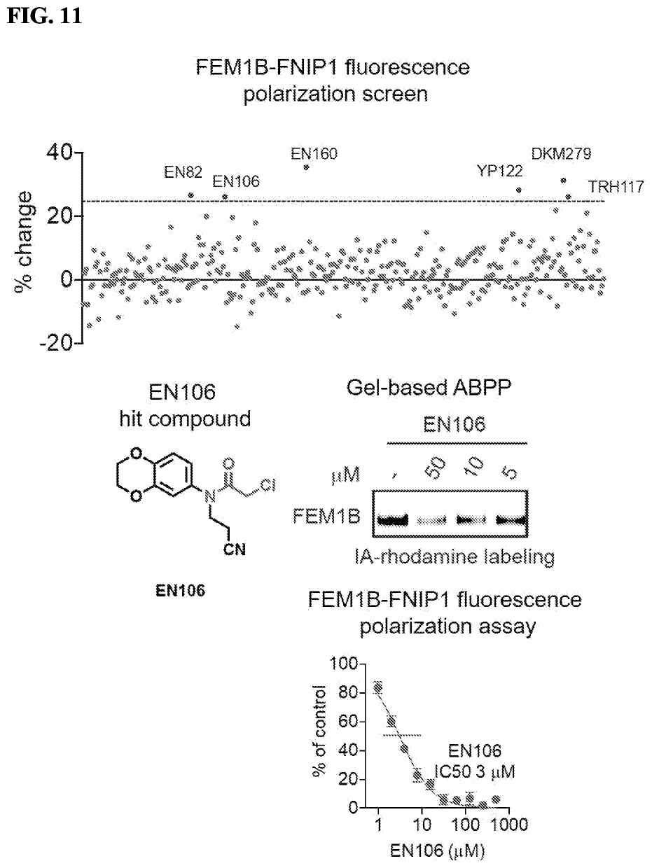

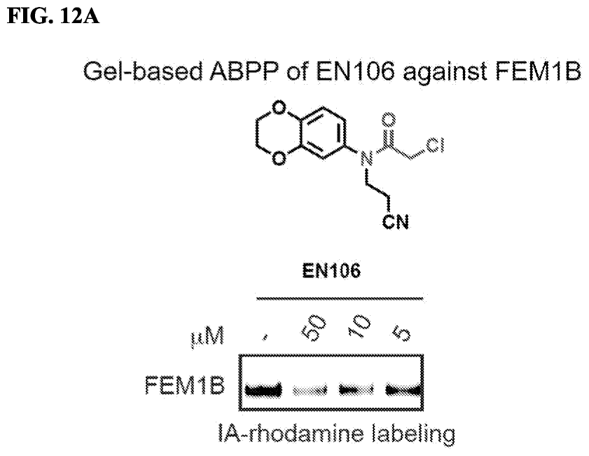

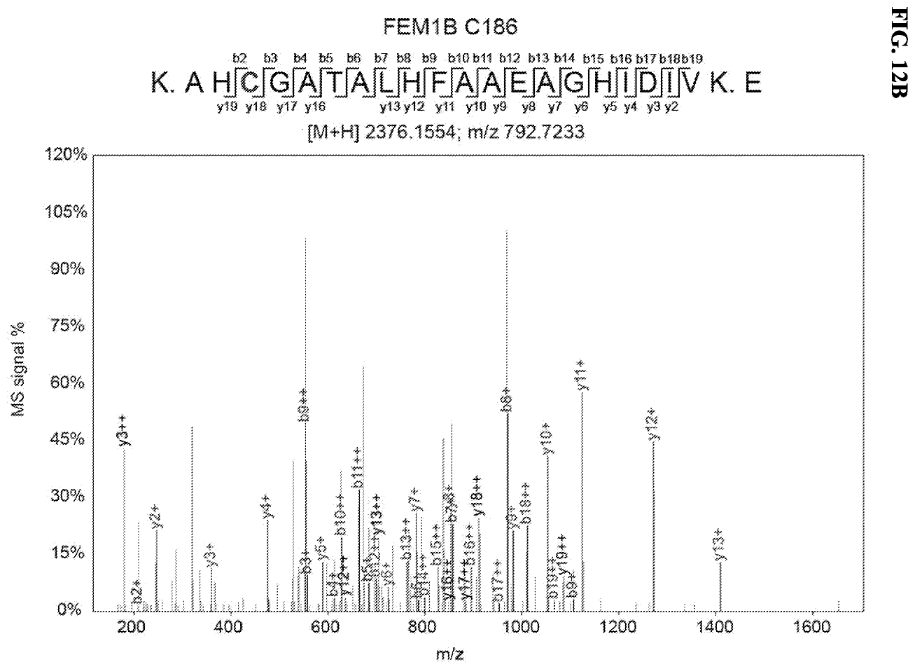

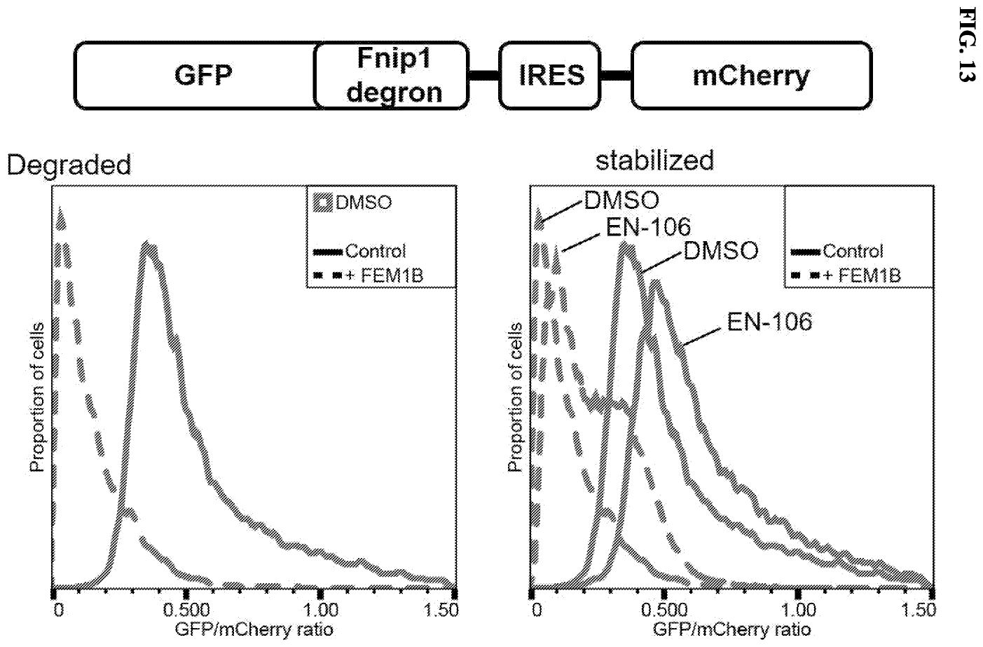

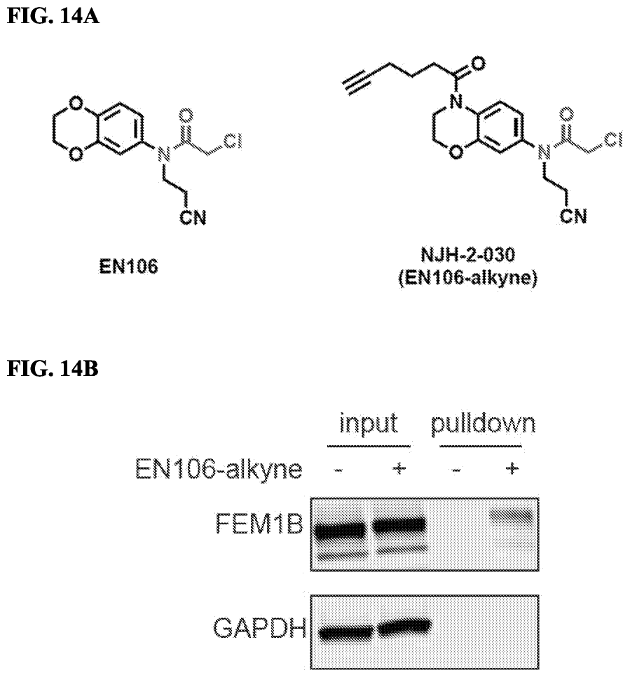

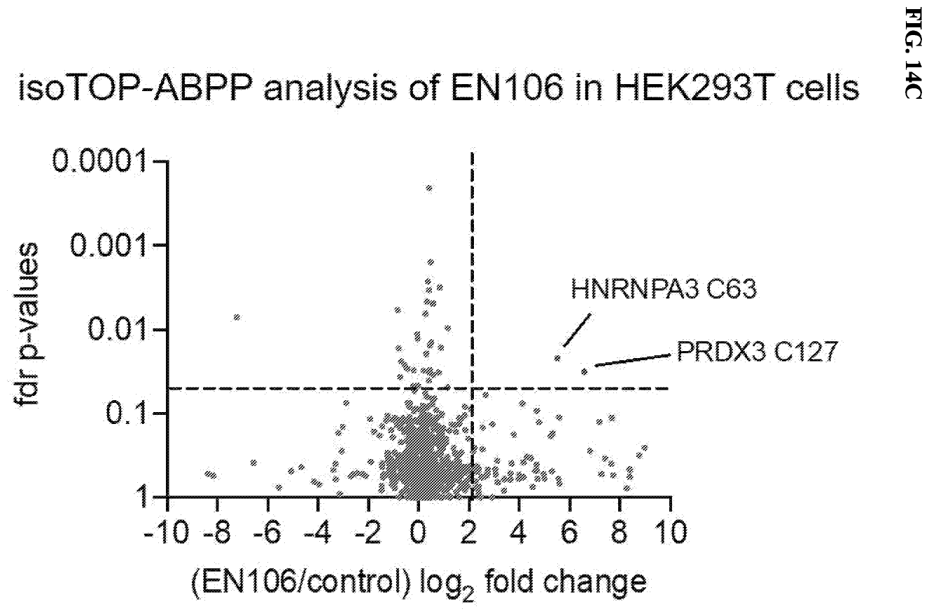

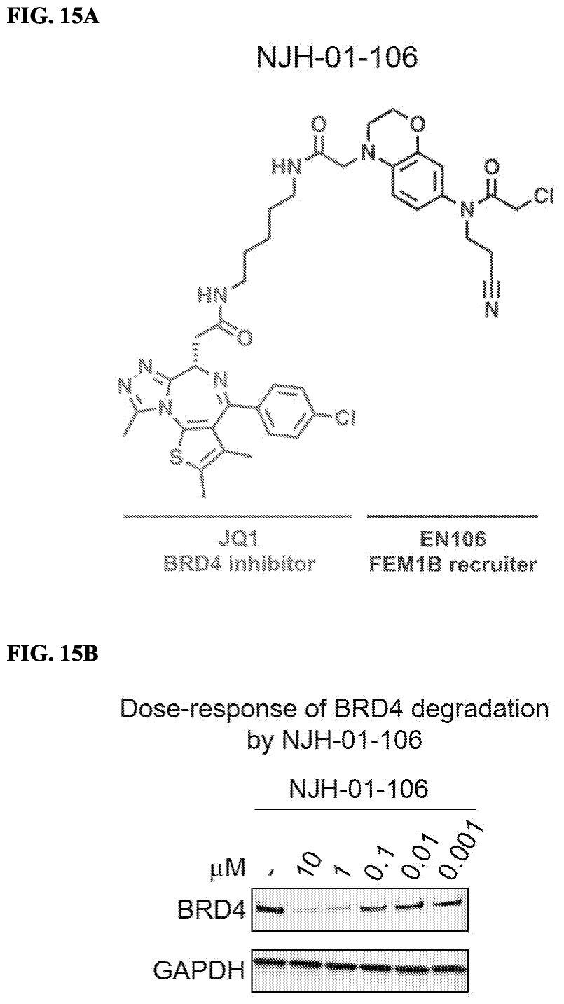

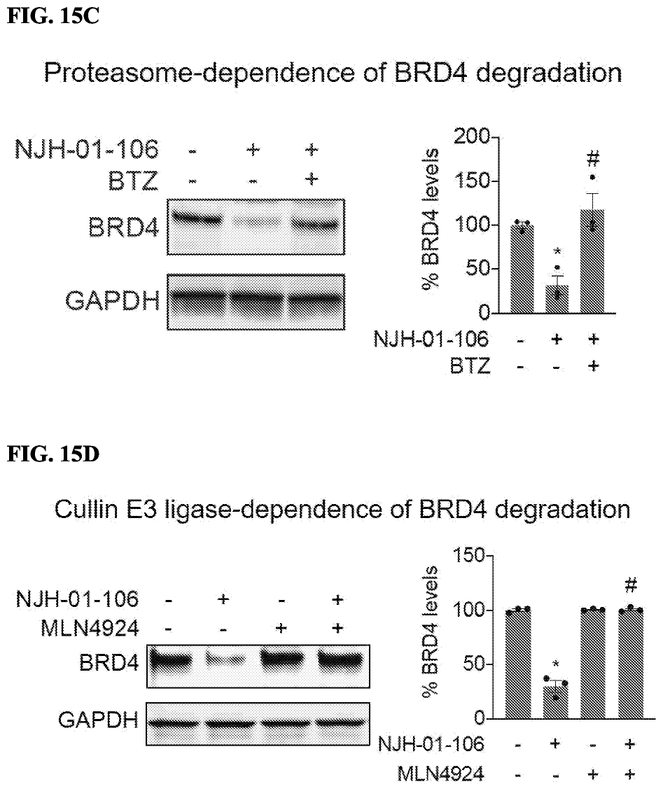

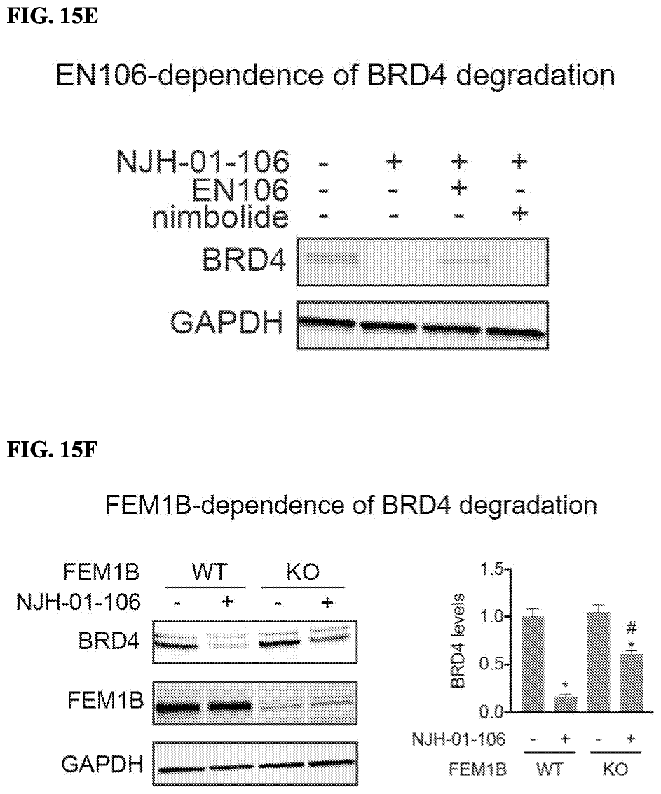

Compound NJH-01-106 (chemical structure shown in FIG. 4) was synthesized and characterized by ¹H NMR and high-resolution mass spectrometry.

¹H NMR (300 MHz, DMSO-d₆) δ 8.20 (t, J=5.6 Hz, 1H), 8.04 (t, J=5.7 Hz, 1H), 7.72 (d, J=4.1 Hz, 1H), 7.52 (d, J=8.4 Hz, 2H), 7.44 (d, J=8.5 Hz, 2H), 6.83-6.77 (m, 1H), 6.55 (d, J=8.9 Hz, 1H), 4.52 (d, J=7.3 Hz, 1H), 4.27 (s, 2H), 4.16 (d, J=5.5 Hz, 1H), 4.02 (s, 2H), 3.90 (s, 2H), 3.80 (t, J=6.6 Hz, 2H), 3.48 (s, 2H), 3.24 (t, J=6.5 Hz, 1H), 3.10 (q, J=6.4 Hz, 4H), 2.69 (t, J=6.6 Hz, 2H), 2.62 (d, J=0.8 Hz, 3H), 2.44 (s, 3H), 1.65 (s, 3H), 1.42 (d, J=22.4 Hz, 4H), 0.96-0.81 (m, 2H).

HRMS (ESI) m/z: [M+H]⁺ calculated 804.2614, found 804.2611.

### Example 2: Degradation of Brd4

231MFP breast cancer cells were treated for 12 hours with NJH-01-106, and 4 hours with known BRD4 PROTAC MZ1. BRD4 and loading control GAPDH levels were assessed by Western blotting following treatment with NJH-01-106 or MZ1, as described above, and are shown in FIG. 4.

### Example 3: Additional Compound Data

TABLE 1
Data for selected compounds.

| Compound | | Normalized FP | Normalized FP |
|---|---|---|---|
| ID | Structure | 50 μM | 5 μM |
| DMSO | | 100 | 100 |
| EN106 | 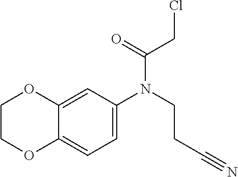 [SMILES: N#CCCN(C(=O)CCl)c1ccc2c(c1)OCCO2] | 4.200 | 50.624 |
| EN-NJH-001 | 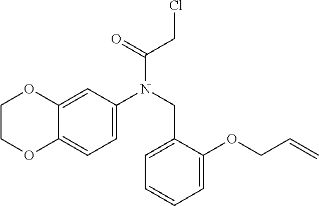 [SMILES: C=CCOc1ccccc1CN(C(=O)CCl)c1ccc2c(c1)OCCO2] | 19.149 | 97.795 |
| EN-NJH-002 | 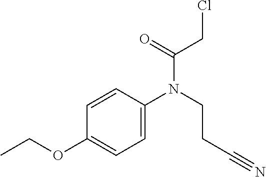 [SMILES: CCOc1ccc(N(CCC#N)C(=O)CCl)cc1] | 8.824 | 40.111 |
| EN-NJH-003 | 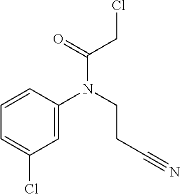 [SMILES: N#CCCN(C(=O)CCl)c1cccc(Cl)c1] | 2.701 | 54.053 |
| EN-NJH-004 | 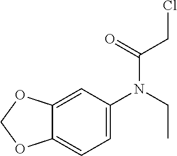 [SMILES: CCN(C(=O)CCl)c1ccc2c(c1)OCO2] | 11.795 | 76.058 |
| EN-NJH-005 | 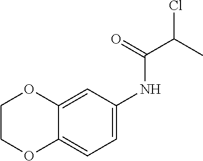 [SMILES: CC(Cl)C(=O)Nc1ccc2c(c1)OCCO2] | 101.387 | 98.107 |
| EN-NJH-006 | 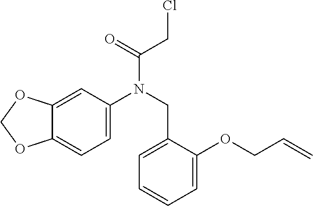 [SMILES: C=CCOc1ccccc1CN(C(=O)CCl)c1ccc2c(c1)OCO2] | 21.467 | 89.666 |
| EN-NJH-007 | 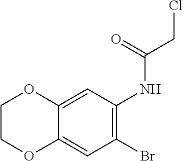 [SMILES: O=C(CCl)Nc1cc2c(cc1Br)OCCO2] | 11.686 | 87.728 |
| EN-NJH-008 | 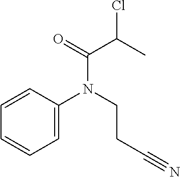 [SMILES: CC(Cl)C(=O)N(CCC#N)c1ccccc1] | 113.342 | 97.906 |
| EN-NJH-009 | 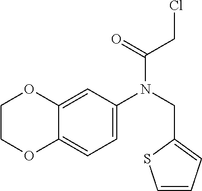 [SMILES: O=C(CCl)N(Cc1cccs1)c1ccc2c(c1)OCCO2] | 5.998 | 74.788 |
| EN-NJH-010 | 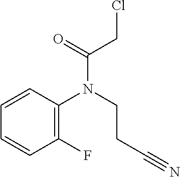 [SMILES: N#CCCN(C(=O)CCl)c1ccccc1F] | 14.584 | 46.793 |
| EN-NJH-011 | 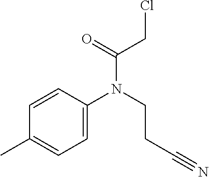 [SMILES: Cc1ccc(N(CCC#N)C(=O)CCl)cc1] | 4.295 | 45.813 |
| EN-NJH-012 | 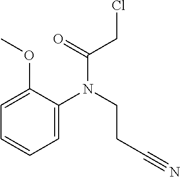 [SMILES: COc1ccccc1N(CCC#N)C(=O)CCl] | 24.837 | 90.401 |
| EN-NJH-013 | 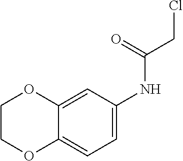 [SMILES: O=C(CCl)Nc1ccc2c(c1)OCCO2] | 21.286 | 93.140 |
| EN-NJH-014 | 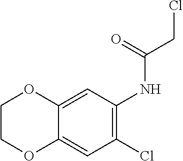 [SMILES: O=C(CCl)Nc1cc2c(cc1Cl)OCCO2] | 21.757 | 94.766 |
| EN-NJH-015 | 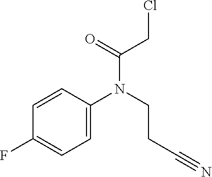 [SMILES: N#CCCN(C(=O)CCl)c1ccc(F)cc1] | 16.287 | 36.281 |
| EN-NJH-016 | 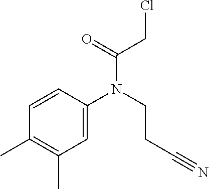 [SMILES: Cc1ccc(N(CCC#N)C(=O)CCl)cc1C] | 11.324 | 43.653 |
| EN-NJH-017 | 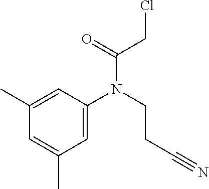 [SMILES: Cc1cc(C)cc(N(CCC#N)C(=O)CCl)c1] | 17.374 | 63.719 |
| EN-NJH-018 | 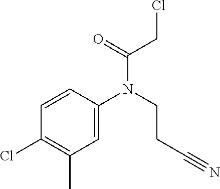 [SMILES: Cc1cc(N(CCC#N)C(=O)CCl)ccc1Cl] | 12.048 | 16.414 |
| EN-NJH-019 | 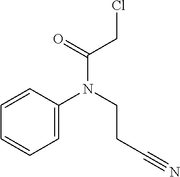 [SMILES: N#CCCN(C(=O)CCl)c1ccccc1] | 19.402 | 44.098 |
| EN-NJH-020 | 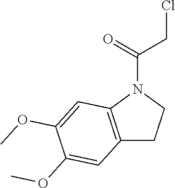 [SMILES: COc1cc2c(cc1OC)N(C(=O)CCl)CC2] | 19.076 | 15.590 |
| EN-NJH-021 | 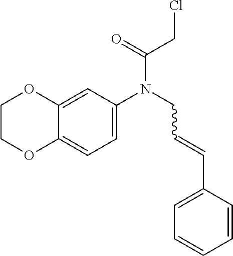 [SMILES: O=C(CCl)N(CC=Cc1ccccc1)c1ccc2c(c1)OCCO2] | 26.829 | 86.437 |
| NJH-01-073 | 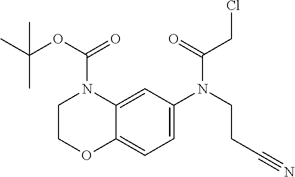 [SMILES: CC(C)(C)OC(=O)N1CCOc2ccc(N(CCC#N)C(=O)CCl)cc21] | 9.874 | 32.762 |
| NJH-01-085 | 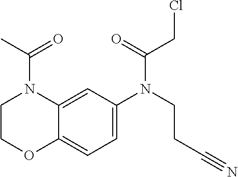 [SMILES: CC(=O)N1CCOc2ccc(N(CCC#N)C(=O)CCl)cc21] | 14.186 | 31.269 |
| SB-02-092 | 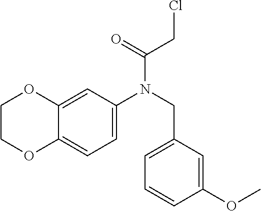 [SMILES: COc1cccc(CN(C(=O)CCl)c2ccc3c(c2)OCCO3)c1] | 12.628 | 54.944 |
| NJH-01-093 | 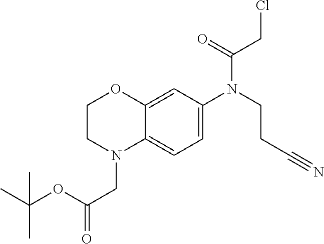 [SMILES: CC(C)(C)OC(=O)CN1CCOc2cc(N(CCC#N)C(=O)CCl)ccc21] | 15.200 | 26.459 |
| NJH-01-098 | 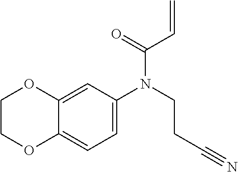 [SMILES: C=CC(=O)N(CCC#N)c1ccc2c(c1)OCCO2] | 115.262 | 104.588 |

FP: fluorescence polarization between FEM1B and FNIP1.

### Example 4: Experimental Procedures and Characterization Data

EN106-alkyne pulldown protocol. HEK293T cells were treated with DMSO or 10 μM NJH-2-030 in situ for 8 h. Cells were harvested, lysed via sonication and normalized to 2.0 mg/mL. Following normalization, 100 μL of each lysate sample was removed for Western blot analysis of input, and 500 μL of each lysate sample was incubated for 1 h at room temperature with 10 μL of 5 mM biotin picolyl azide (in water) (Sigma Aldrich 900912), 10 μL of 50 mM TCEP (in water), 30 μL of TBTA ligand (0.9 mg/mL in DMSO:t-butanol=1:4), and 10 μL of 50 mM Copper (II) Sulfate (12.5 mg/mL in water). Proteins were precipitated, washed 3×1 mL with cold MeOH, resolubilized in 1 mL of 1.2% SDS/PBS (w/v), heated for 5 min at 90° C., and centrifuged to remove any insoluble components. 1 mL of each resolubilized sample was then transferred to 15 mL conical tubes containing 5 mL PBS with 85 μL streptavidin resin (Thermo Scientific 53114) to give a final SDS concentration of 0.2%. Samples were incubated with the streptavidin beads at 4° C. overnight on a rotator. The following day the samples were warmed to room temperature and washed with 0.2% SDS, then transferred to spin columns and further washed 3× with 500 μL PBS and 3× with 500 μL water to remove non-probe-labeled proteins. The washed beads were resuspended in 100 μL PBS, transferred to 1.5 mL eppendorflow-adhesion tubes, combined with 30 μL Laemmli Sample Buffer (4×) and heated to 95° C. Proteins in each sample were then analyzed by Western blotting to look for enriched FEM1B versus non-enriched GAPDH.

IsoTOP-ABPP Chemoproteomic Experiment. IsoTOP-ABPP was performed as previously reported (Grossman et al., 2017; Spradlin et al., 2019; Weerapana et al., 2010). Cells were lysed by probe sonication in PBS and protein concentrations were measured by BCA assay. Cells were treated for 2 h with either DMSO vehicle or 10 μM EN106 (from 1,000× DMSO stock) before cell collection and lysis. Proteomes were subsequently labeled with IA-alkyne labeling (100 μM) for 1 h at room temperature. CuAAC was used by sequential addition of tris(2-carboxyethyl)phosphine (1 mM, Strem, 15-7400), tris[(1-benzyl-1H-1,2,3-triazol-4-yl)methyl]amine (34 μM, Sigma, 678937), copper(II) sulfate (1 mM, Sigma, 451657) and biotin-linker-azide—the linker functionalized with a tobacco etch virus (TEV) protease recognition sequence as well as an isotopically light or heavy valine for treatment of control or treated proteome, respectively. After CuAAC, proteomes were precipitated by centrifugation at 6,500 g, washed in ice-cold methanol, combined in a 1:1 control:treated ratio, washed again, then denatured and resolubilized by heating in 1.2% SDS PBS to 80° C. for 5 min. Insoluble components were precipitated by centrifugation at 6,500 g and soluble proteome was diluted in 5 ml 0.2% SDS-PBS. Labeled proteins were bound to streptavidin-agarose beads (170 ml resuspended beads per sample, Thermo Fisher, 20349) while rotating overnight at 4° C. Bead-linked proteins were enriched by washing three times each in PBS and water, then resuspended in 6 M urea in PBS, and reduced in TCEP (1 mM, Strem, 15-7400), alkylated with iodoacetamide (18 mM, Sigma), before being washed and resuspended in 2 M urea in PBS and trypsinized overnight with 0.5 μg/μL sequencing grade trypsin (Promega, V5111). Tryptic peptides were eluted off. Beads were washed three times each in PBS and water, washed in TEV buffer solution (water, TEV buffer, 100 μM dithiothreitol) and resuspended in buffer with Ac-TEV protease (Invitrogen, 12575-015) and incubated overnight. Peptides were diluted in water and acidified with formic acid (1.2 M, Fisher, A117-50) and prepared for analysis.

IsoTOP-ABPP Mass Spectrometry Analysis. Peptides from all chemoproteomic experiments were pressure-loaded onto a 250 μm inner diameter fused silica capillary tubing packed with 4 cm of Aqua C18 reverse-phase resin (Phenomenex, 04A-4299), which was previously equilibrated on an Agilent 600 series high-performance liquid chromatograph using the gradient from 100% buffer A to 100% buffer B over 10 min, followed by a 5 min wash with 100% buffer B and a 5 min wash with 100% buffer A. The samples were then attached using a MicroTee PEEK 360 μm fitting (Thermo Fisher Scientific p-888) to a 13 cm laser pulled column packed with 10 cm Aqua C18 reverse-phase resin and 3 cm of strong-cation exchange resin for isoTOP-ABPP studies. Samples were analyzed using an Q Exactive Plus mass spectrometer (Thermo Fisher Scientific) using a five-step Multidimensional Protein Identification Technology (MudPIT) program, using 0, 25, 50, 80 and 100% salt bumps of 500 mM aqueous ammonium acetate and using a gradient of 5-55% buffer B in buffer A (buffer A: 95:5 water:acetonitrile, 0.1% formic acid; buffer B 80:20 acetonitrile:water, 0.1% formic acid). Data were collected in data-dependent acquisition mode with dynamic exclusion enabled (60 s). One full mass spectrometry (MS1) scan (400-1,800 mass-to-charge ratio (m/z)) was followed by 15 MS2 scans of the nth most abundant ions. Heated capillary temperature was set to 200° C. and the nanospray voltage was set to 2.75 kV. Data were extracted in the form of MS1 and MS2 files using Raw Extractor v.1.9.9.2 (Scripps Research Institute) and searched against the Uniprot human database using ProLuCID search methodology in IP2 v.3 (Integrated Proteomics Applications, Inc.) (Xu et al., 2015). Cysteine residues were searched with a static modification for carboxyaminomethylation (+57.02146) and up to two differential modifications for methionine oxidation and either the light or heavy TEV tags (+464.28596 or +470.29977, respectively). Peptides were required to be fully tryptic peptides and to contain the TEV modification. ProLUCID data were filtered through DTASelect to achieve a peptide false-positive rate below 5%. Only those probe-modified peptides that were evident across two out of three biological replicates were interpreted for their isotopic light to heavy ratios. For those probe-modified peptides that showed ratios greater than two, we only interpreted those targets that were present across all three biological replicates, were statistically significant and showed good quality MS1 peak shapes across all biological replicates. Light versus heavy isotopic probe-modified peptide ratios were calculated by taking the mean of the ratios of each replicate paired light versus heavy precursor abundance for all peptide-spectral matches associated with a peptide. The paired abundances were also used to calculate a paired sample t-test P value in an effort to estimate constancy in paired abundances and significance in change between treatment and control. P values were corrected using the Benjamini-Hochberg method.

Cell Lysis Protocol. Pelleted cells were lysed with RIPA lysis buffer (50 mM Tris-HCl, 165 mM NaCl, 12 mM sodium deoxycholate, 1% Triton X-100, 0.01% SDS), protein concentration normalized using a BCA assay (Thermo).

Western Blot Protocol. Proteins were resolved by SDS-PAGE (4-20% TGX gels, Bio-Rad Laboratories, Inc.) and transferred to nitrocellulose membranes using the Trans-Blot Turbo transfer system (Bio-Rad). Membranes were blocked with 5% BSA in Tris-buffered saline containing Tween 20 (TBST) solution for 1 h at room temperature, washed in TBST and probed with primary antibody diluted in diluent, as recommended by the various manufacturers, overnight at 4° C. Primary antibodies used were: BRD4 (Cell Signaling Technologies #13440), GAPDH (ProteinTech 60004-1-Ig), FEM1B (ProteinTech 19544-1-AP). Following washes with TBST, the blots were incubated in the dark with secondary antibodies purchased from Li-Cor Biosciences and used at 1:10,000 dilution in 5% BSA in TBST at room temperature for 1 h. Blots were visualized using an Odyssey Li-Cor scanner after additional washes. Protein intensity was quantified using ImageJ software.

Cell Culture. HEK293T cells were obtained from the UC Berkeley Cell Culture Facility and cultured in DMEM (Gibco) containing 10% (v/v) fetal bovine serum (FBS) and maintained at 37° C. with 5% CO₂. The FEM1B knockout HEK293T cell line was generated as described by Manford et al. (Cell 183, 1-16, Oct. 1, 2020).

### Synthesis of EN 106

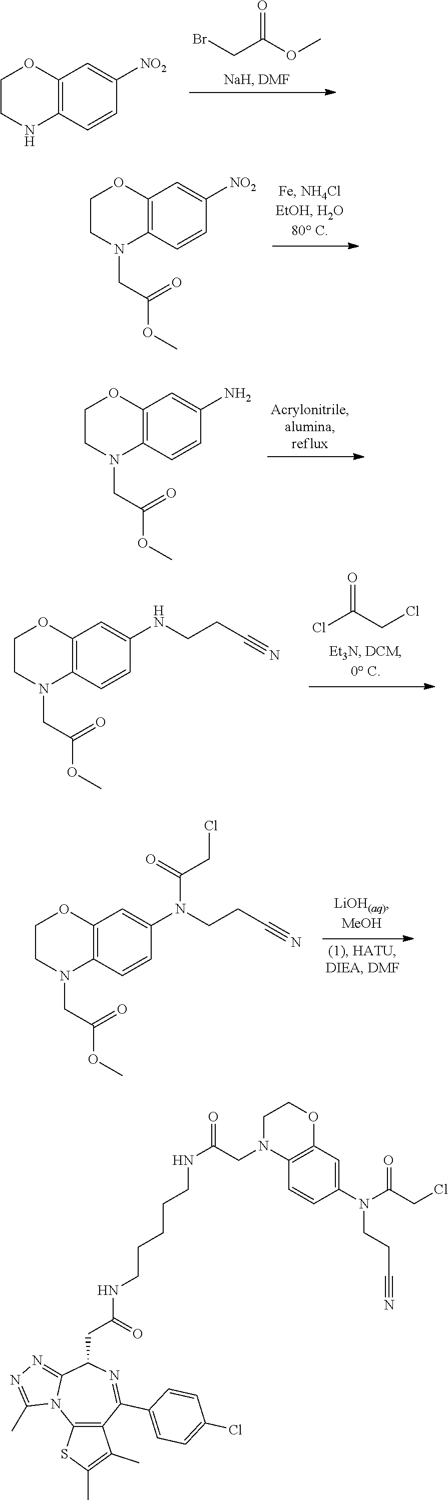

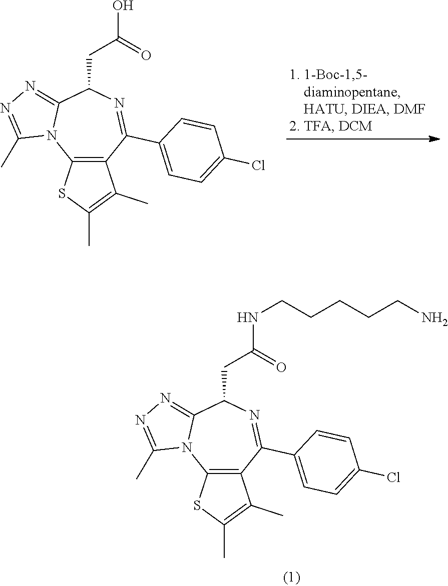 [SMILES: Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCCCCN)c1nnc(C)n1-2]

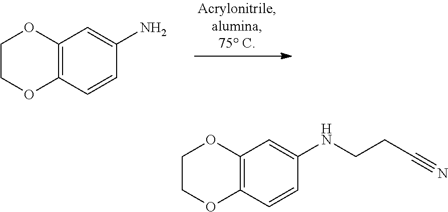 [SMILES: Nc1ccc2c(c1)OCCO2 ; N#CCCNc1ccc2c(c1)OCCO2]

3-((2,3-dihydrobenzo[b][1,4]dioxin-6-yl)amino)propanenitrile: 2,3-dihydrobenzo[b][1,4]dioxin-6-amine (302 mg, 2.0 mmol) was dissolved in acrylonitrile (1.3 mL) and basic alumina (204 mg, 2.0 mmol) was added. The mixture was stirred at 75° C. for 18 hours before being filtered through Celite. The filtrate was concentrated, and the residue purified by silica gel chromatography to obtain the title compound (321 mg, 1.57 mmol, 79%) as an amber oil. LCMS [M+H]⁺ calc 205.09, found 205.1. ¹H NMR (500 MHz, CDCl₃) δ 6.75 (dd, J=8.1, 0.8 Hz, 1H), 6.22-6.16 (m, 2H), 4.29-4.19 (m, 4H), 3.65 (s, 1H), 3.47 (t, J=6.5 Hz, 2H), 2.64 (t, J=6.5 Hz, 2H).

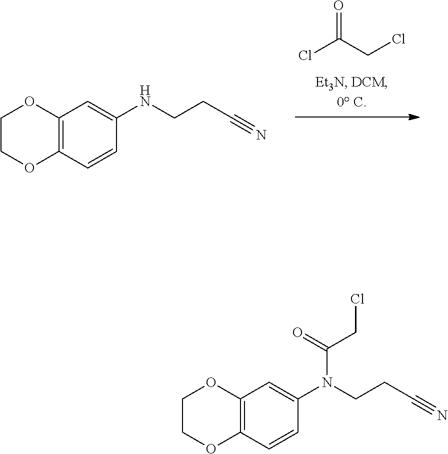 [SMILES: O=C(Cl)CCl ; N#CCCNc1ccc2c(c1)OCCO2 ; N#CCCN(C(=O)CCl)c1ccc2c(c1)OCCO2]

2-chloro-N-(2-cyanoethyl)-N-(2,3-dihydrobenzo[b][1,4]dioxin-6-yl)acetamide (EN106): 3-((2,3-dihydrobenzo[b][1,4]dioxin-6-yl)amino)propanenitrile (42 mg, 0.21 mmol) was dissolved in DCM (2 mL), and TEA (58 μL, 0.42 mmol) was added followed by chloroacetyl chloride (22 μL, 0.27 mmol), dropwise at 0° C. The solution was stirred for 20 minutes at 0° C., water was added, the mixture partitioned, and the aqueous layer extracted with DCM. Combined organic extracts were washed with brine, dried over Na₂SO₄, concentrated, and the crude residue purified by silica gel chromatography and lyophilized to obtain the title compound (34 mg, 0.12 mmol, 58%) as a white solid. LCMS [M+H]⁺ calc 281.06, found 281.1. ¹H NMR (300 MHz, DMSO) δ 7.02 (d, J=2.4 Hz, 1H), 6.98 (d, J=8.5 Hz, 1H), 6.91 (d, J=2.5 Hz, 1H), 4.30 (s, 4H), 4.05 (s, 2H), 3.86 (t, J=6.6 Hz, 2H), 2.72 (t, J=6.6 Hz, 2H).

### Synthesis of NJH-01-106

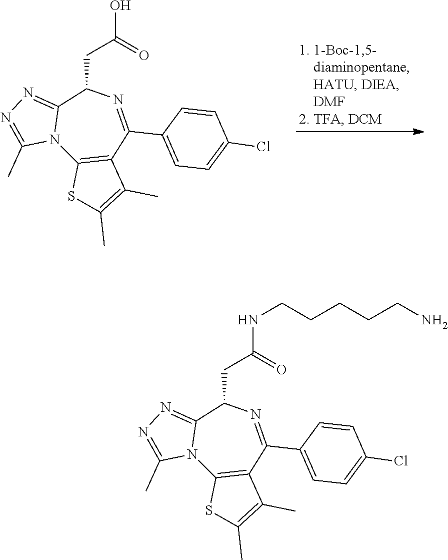 [SMILES: Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCCCCN)c1nnc(C)n1-2]

(S)—N-(5-aminopentyl)-2-(4-(4-chlorophenyl)-2,3,9-trimethyl-6H-thieno[3,2-f][1,2,4]triazolo[4,3-a][1,4]diazepin-6-yl)acetamide (Intermediate 1): (S)-2-(4-(4-chlorophenyl)-2,3,9-trimethyl-6H-thieno[3,2-f][1,2,4]triazolo[4,3-a][1,4]diazepin-6-yl)acetic acid (50 mg, 0.13 mmol) and 1-Boc-1,5-diaminopentane were dissolved in DMF (2 mL). DIEA (113 μL, 0.65 mmol) was added, followed by HATU (100 mg, 0.26 mmol) at rt and the solution was stirred for 20 minutes. Water was added and the mixture extracted with EtOAc, combined organic extracts were washed with brine, dried over Na₂SO₄, concentrated and the crude residue was purified by silica gel chromatography to provide the Boc-protected amine as a colorless oil. LCMS [M+H]⁺ calc 585.2, found 585.2. This oil was then dissolved in DCM (0.5 mL) and TFA (0.5 mL) was added and the solution stirred for 2 hours before volatiles were evaporated, the residue redissolved in DCM and this solution washed with aqueous sat. NaHCO₃. The DCM was then evaporated to provide the title compound as a colorless oil (31 mg, 0.063 mmol, 48% over two steps). LCMS [M+H]⁺ calc 485.2, found 485.2.

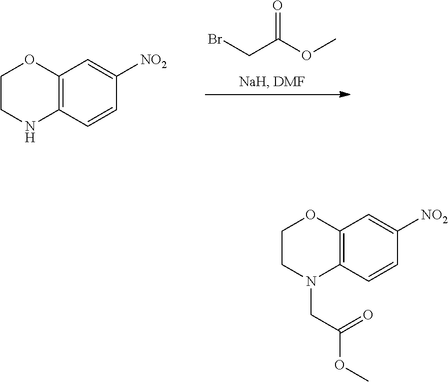

Methyl 2-(7-nitro-2,3-dihydro-4H-benzo[b][1,4]oxazin-4-yl)acetate: 7-nitro-3,4-dihydro-2H-benzo[b][1,4]oxazine (1.0 g, 5.55 mmol) was dissolved in DMF (20 mL) and cooled to 0° C. NaH (233 mg, 5.83 mmol, 60% in mineral oil) was then added to the solution portionwise, and allowed to stir at 0° C. for 30 minutes before methyl bromoacetate (650 μL, 4.43 mmol) was added dropwise. The solution was then allowed to warm to room temperature and stirred for 2 hours, before it was again cooled to 0° C. and diluted with water (80 mL). The resulting suspension was filtered to obtain the title compound (1.32 g, 5.23 mmol, 94%) as a bright yellow powder. LCMS [M+H]⁺ calc 253.07, found 253.2. ¹H NMR (300 MHz, CDCl₃) δ 7.83 (d, J=9.2 Hz, 1H), 7.75 (s, 1H), 6.50 (d, J=8.8 Hz, 1H), 4.34 (s, 2H), 4.17 (d, J=1.9 Hz, 2H), 3.81 (s, 3H), 3.62 (s, 2H).

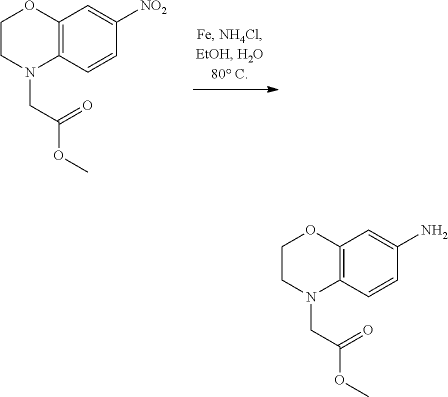 [SMILES: COC(=O)CN1CCOc2cc([N+](=O)[O-])ccc21 ; COC(=O)CN1CCOc2cc(N)ccc21]

Methyl 2-(7-amino-2,3-dihydro-4H-benzo[b][1,4]oxazin-4-y)acetate: methyl 2-(7-nitro-2,3-dihydro-4H-benzo[b][1,4]oxazin-4-yl)acetate (1.32 g, 5.23 mmol) was dissolved in EtOH (30 mL) and water (8 mL) before NH4CL (1.68 g, 31.4 mmol) was added. The mixture was heated to 60° C., Fe powder (876 mg, 15.7 mmol) was added, and the mixture heated at 80C for 17 hours. The reaction mixture was filtered through Celite, and the celite pad washed with EtOAc. Water was added to the filtrate and the mixture extracted with EtOAc. Combined organic extracts were washed with brine, dried over Na₂SO₄, and concentrated to provide the title compound (270 mg, 1.0 mmol, 102%) as a brown oil without further purification. LCMS [M+H]⁺ calc 223.1, found 223.1. 1H NMR (300 MHz, DMSO) δ 6.30 (d, J=8.3 Hz, 1H), 6.09-6.03 (m, 2H), 4.49 (d, J=15.6 Hz, 2H), 4.15 (s, 2H), 4.05 (d, J=10.8 Hz, 2H), 3.63 (d, J=2.0 Hz, 3H), 3.37 (s, 2H).

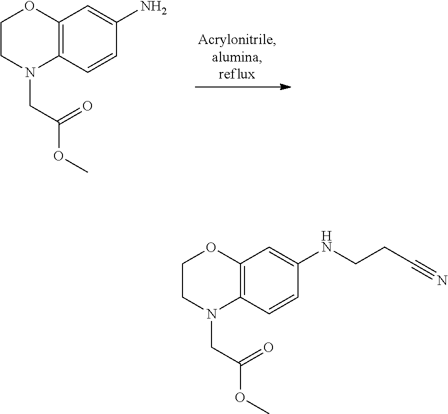 [SMILES: COC(=O)CN1CCOc2cc(N)ccc21 ; COC(=O)CN1CCOc2cc(NCCC#N)ccc21]

Methyl 2-(7-((2-cyanoethyl)amino)-2,3-dihydro-4H-benzo[b][1,4]oxazin-4-yl)acetate: methyl 2-(7-amino-2,3-dihydro-4H-benzo[b][1,4]oxazin-4-yl)acetate (974 mg, 4.20 mmol) was dissolved in acrylonitrile (15 mL) and basic alumina (857 mg, 8.40 mmol) was added and the mixture stirred at 80° C. for 36 h. The reaction mixture was then diluted with EtOAc and filtered through Celite. Water was added to the filtrate, the mixture partitioned, and the aqueous layer extracted with EtOAc. Combined organic extracts were washed with brine, dried over Na₂SO₄, concentrated, and the crude residue purified by silica gel chromatography to obtain the title compound (886 mg, 3.22 mmol, 77%) as an amber oil. LCMS [M+H]⁺ calc 276.13, found 276.1.

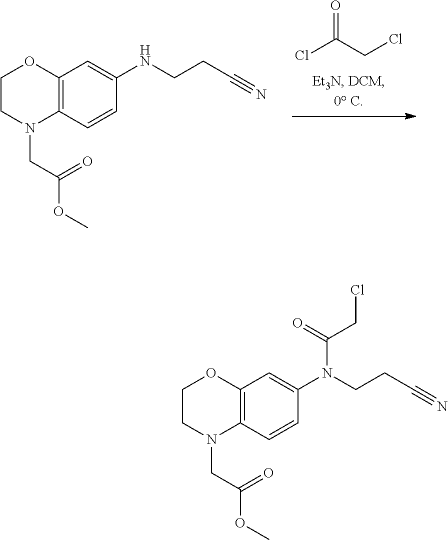 [SMILES: O=C(Cl)CCl ; COC(=O)CN1CCOc2cc(NCCC#N)ccc21 ; COC(=O)CN1CCOc2cc(N(CCC#N)C(=O)CCl)ccc21]

Methyl 2(7-(2-chloro-N-(2-cyanoethyl)acetamido)-2,3-dihydro-4H-benzo[b][1,4]oxazin-4-yl)acetate: methyl 2-(7-((2-cyanoethyl)amino)-2,3-dihydro-4H-benzo[b][1,4]oxazin-4-yl)acetate (215 mg, 0.78 mmol) was dissolved in DCM (4 mL). The solution was cooled to 0° C. and TEA (326 uL, 2.34 mmol) was added, followed by chloroacetyl chloride (92 uL, 1.17 mmol). After 15 mintues at 0° C., the reaction mixture was concentrated and the crude residue purified by silica gel chromatography to obtain the title compound (229 mg, 0.65 mmol, 84%) as amber oil. LCMS [M+H]*calc 352.1, found 352.1. ¹H NMR (400 MHz, DMSO) δ 6.82-6.72 (m, 2H), 6.62 (dd, J=8.5, 2.1 Hz, 1H), 4.29-4.18 (m, 4H), 4.08-3.98 (m, 3H), 3.80 (t, J=6.7 Hz, 2H), 3.66 (d, J=2.2 Hz, 3H), 3.44 (s, 2H), 2.68 (t, J=6.7 Hz, 2H).

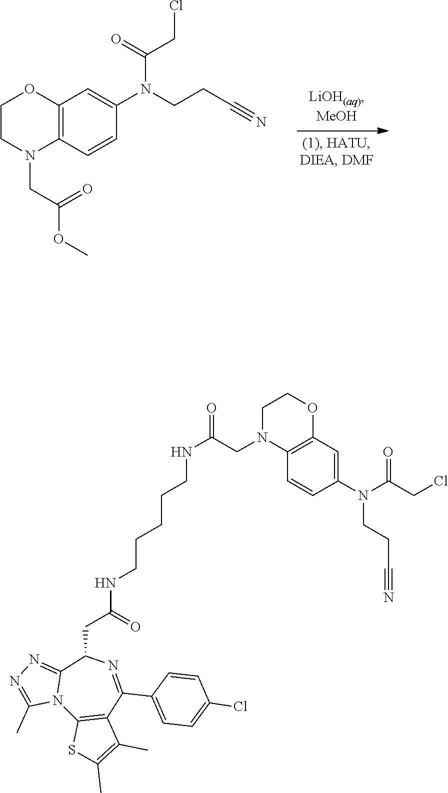 [SMILES: COC(=O)CN1CCOc2cc(N(CCC#N)C(=O)CCl)ccc21 ; Cc1sc2c(c1C)C(c1ccc(Cl)cc1)=N[C@@H](CC(=O)NCCCCCNC(=O)CN1CCOc3cc(N(CCC#N)C(=O)CCl)ccc31)c1nnc(C)n1-2]

(S)-2-chloro-N-(4-(2-((5-(2-(4-(4-chlorophenyl)-2,3,9-trimethyl-6H-thieno[3,2-f][1,2,4]triazolo[4,3-a][1,4]diazepin-6-yl)acetamido)pentyl)amino)-2-oxoethyl)-3,4-dihydro-2H-benzo[b][1,4]oxazin-7-yl)-N-(2-cyanoethyl)acetamide (NJH-01-106): methyl 2-(7-(2-chloro-N-(2-cyanoethyl)acetamido)-2,3-dihydro-4H-benzo[b][1,4]oxazin-4-yl)acetate (22 mg, 0.064 mmol) was dissolved in MeOH (600 μL) and treated with aqueous LiOH (400 uL, 0.5 M, 0.2 mmol) at rt for 1 h. The mixture diluted with DCM (5 mL) and acidified with aqueous HCl (400 μL, 1 M) and the mixture extracted with DCM. Combined organic extracts were dried over Na₂SO₄, concentrated, and the crude residue dissolved in DMF (0.5 mL). DIEA (55 μL, 0.32 mmol) was added followed by (S)—N-(5-aminopentyl)-2-(4-(4-chlorophenyl)-2,3,9-trimethyl-6H-thieno[3,2-f][1,2,4]triazolo[4,3-a][1,4]diazepin-6-yl)acetamide (Intermediate 1, 31 mg, 0.064 mmol) then HATU (49 mg, 0.13 mmol). The resulting solution was stirred for 10 minutes before it was diluted with EtOAc (0.5 mL) and purified by silica gel chromatography (0-10% MeOH/DCM) followed by preparatory this layer chromatography to obtain the title compound (26 mg, 0.032 mmol, 50%) as a white lyophilized solid. HRMS [M+H]⁺ calc 804.2535, found 804.2669. ¹H NMR (400 MHz, CDCl₃) δ 7.44-7.31 (m, 4H), 7.04-6.96 (m, 1H), 6.74 (dd, J=8.5, 2.5 Hz, 2H), 6.68 (d, J=2.5 Hz, 1H), 6.59 (d, J=8.5 Hz, 1H), 4.62 (dd, J=8.8, 5.3 Hz, 1H), 4.30 (t, J=4.5 Hz, 2H), 3.89 (d, J=5.5 Hz, 6H), 3.55 (dd, J=14.4, 8.8 Hz, 1H), 3.46 (dq, J=7.7, 3.8, 3.2 Hz, 11H), 3.43-3.32 (m, 111), 3.32-3.24 (m, 111), 3.23-3.13 (m, 211), 2.69 (dd, J=6.9, 2.9 Hz, 2H), 2.67 (s, 3H), 2.41 (d, J=0.9 Hz, 311), 1.70-1.65 (m, 311), 1.55-1.41 (m, 611), 1.35 (h, J=6.0, 5.5 Hz, 2H). ¹³C NMR (151 MHz, CDCl₃) δ 170.49, 169.20, 167.15, 164.08, 155.65, 149.90, 145.04, 136.92, 136.55, 135.77, 132.05, 131.07, 130.96, 130.94, 130.48, 129.85, 128.78, 121.05, 117.68, 115.75, 113.37, 64.79, 56.08, 55.58, 54.53, 48.69, 46.11, 41.91, 39.32, 39.15, 39.04, 28.70, 23.78, 18.65, 17.30, 16.28, 14.38, 13.11, 11.81.

### Synthesis of EN106-Alkyne

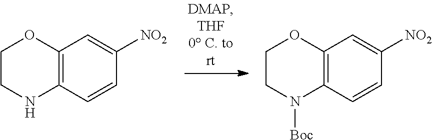 [SMILES: O=[N+]([O-])c1ccc2c(c1)OCCN2 ; CC(C)(C)OC(=O)N1CCOc2cc([N+](=O)[O-])ccc21]

tert-Butyl 7-nitro-2,3-dihydro-4H-benzo[b][1,4]oxazine-4-carboxylate: 7-nitro-3,4-dihydro-2H-benzo[b][1,4]oxazine (1.0 g, 5.55 mmol) was dissolved in THF (20 mL) and DMAP (67 mg, 0.55 mmol) was added at 0° C. followed by Boc₂O (1.45 g, 6.66 mmol). The ice bath was removed after 5 minutes and the mixture stirred overnight at room temperature. The reaction was basified with 15 mL 1 M NaOH, stirred for 2 hours, concentrated, and the aqueous mixture extracted with EtOAc. Organic extracts were washed with 1 M HCl, brine, dried over Na₂SO₄, and concentrated to provide the title compound as an orange solid (1.65 g, 5.89 mmol, 106%). LCMS [M+H]r calc 281.11, found 281.1. ¹H NMR (300 MHz, CDCl₃) δ 8.11 (d, J=9.1 Hz, 1H), 7.81 (s, 2H), 4.35 (s, 2H), 3.96 (s, 2H), 1.61 (s, 9H).

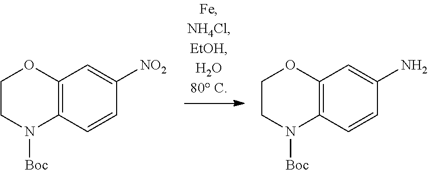 [SMILES: CC(C)(C)OC(=O)N1CCOc2cc([N+](=O)[O-])ccc21 ; CC(C)(C)OC(=O)N1CCOc2cc(N)ccc21]

tert-Butyl 7-amino-2,3-dihydro-4H-benzo[b][1,4]oxazine-4-carboxylate: tert-butyl 7-nitro-2,3-dihydro-4H-benzo[b][1,4]oxazine-4-carboxylate (5.55 mmol) was dissolved in EtOH (20 mL) and water (5 mL) before NH₄Cl (1.78 g, 33.3 mmol) was added. The mixture was heated to 60° C., Fe powder (932 mg, 16.7 mmol) was added, and the mixture heated at 80° C. for 13 hours. The reaction was filtered through Celite, and celite washed with EtOAc. Water was added to the filtrate and the mixture extracted with EtOAc. Combined organic extracts were washed with brine, dried over Na₂SO₄, and concentrated to provide the title compound as an orange oil (1.41 g, 102% over two steps). LCMS [M+H]⁺ calc 251.1, found 251.1. ¹H NMR (300 MHz, CDCl₃) δ 7.54 (s, 1H), 6.33-6.21 (m, 2H), 4.24 (s, 2H), 3.85 (s, 2H), 3.58 (s, 2H), 1.56 (s, 9H).

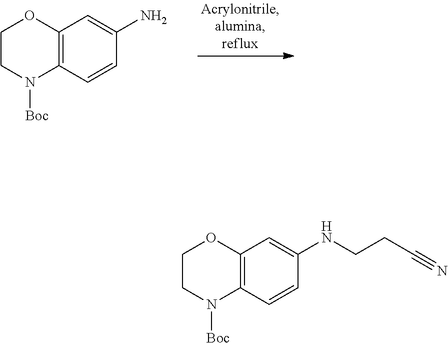 [SMILES: CC(C)(C)OC(=O)N1CCOc2cc(N)ccc21 ; CC(C)(C)OC(=O)N1CCOc2cc(NCCC#N)ccc21]

tert-Butyl7-((2-cyanoethyl)amino)-2,3-dihydro-4H-benzo[b][1,4]oxazine-4-carboxylate: tert-butyl 7-amino-2,3-dihydro-4H-benzo[b][1,4]oxazine-4-carboxylate (1.41 g, 5.66 mmol) was dissolved in acrylonitrile (20 mL). Alumina (1.13 g, 11.1 mmol) was added and the mixture refluxed for 48 hours, before being diluted with EtOAc and filtered through Celite to remove alumina. The filtrate was concentrated to provide the title compound as an orange oil (1.82 g, 6.00 mmol, 108% over 3 steps) with a minor double alkylated impurity. LCMS [M+H]⁺ calc 304.2, found 304.2.

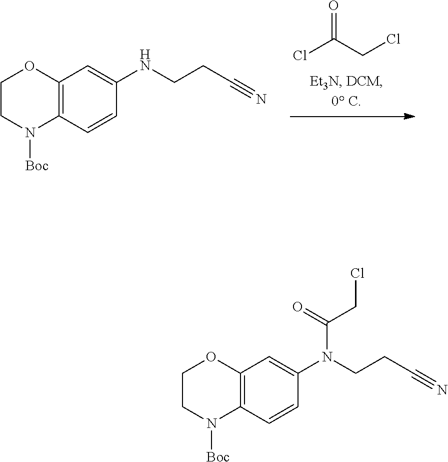 [SMILES: O=C(Cl)CCl ; CC(C)(C)OC(=O)N1CCOc2cc(NCCC#N)ccc21 ; CC(C)(C)OC(=O)N1CCOc2cc(N(CCC#N)C(=O)CCl)ccc21]

tert-Butyl 7-(2-chloro-N-(2-cyanoethyl)acetamido)-2,3-dihydro-4H-benzo[b][1,4]oxazine-4-carboxylate: tert-butyl 7-((2-cyanoethyl)amino)-2,3-dihydro-4H-benzo[b][1,4]oxazine-4-carboxylate was dissolved in DCM (6 mL), the solution cooled to 0° C., and TEA (556 μL, 4 mmol) was added followed by chloroacetyl chloride (202 μL, 2.5 mmol). The solution was stirred at 0° C. for 5 minutes and allowed to warm to rt over 1 hour. Aqueous NaHCO₃ was added, the mixture partitioned, the aqueous layer extracted with DCM. Combined organic extracts were dried over sodium sulfate, concentrated and purified by silica gel chromatography to obtain the title compound (244 mg, 0.64 mmol, 64%) as an orange solid. LCMS [M+H]⁺ calc 380.1, found 308.1. ¹H NMR (400 MHz, CDCl₃) δ 8.01 (s, 1H), 6.87-6.79 (m, 2H), 4.35-4.28 (m, 2H), 4.06-3.89 (m, 6H), 2.75 (t, J=6.8 Hz, 2H), 1.60 (s, 9H).

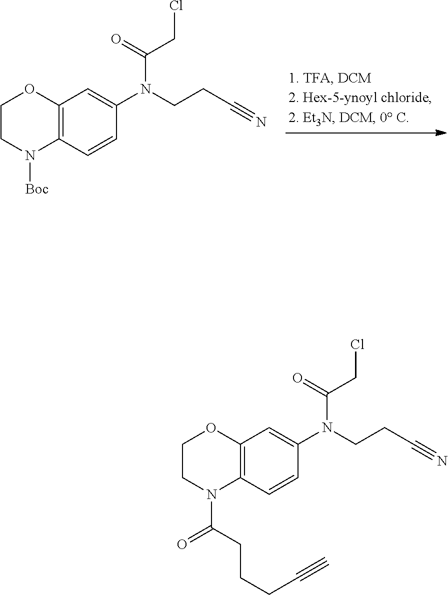 [SMILES: CC(C)(C)OC(=O)N1CCOc2cc(N(CCC#N)C(=O)CCl)ccc21 ; C#CCCCC(=O)N1CCOc2cc(N(CCC#N)C(=O)CCl)ccc21]

2-Chloro-N-(2-cyanoethyl)-N-(4-(hex-5-ynoyl),3,4-dihydro-2H-benzo[b][1,4]oxazin-7-yl)acetamide (EN106-alkyne): Oxalyl chloride (150 μL of 2.0 M solution in DCM, 0.3 mmol) was added to solution of 5-hexynoic acid (17 μL, 0.15 mmol) and 1 drop of DMF in 1 mL in DCM at room temperature. The solution was stirred for 30 minutes before being concentrated to give crude hex-5-ynoyl chloride as a pink foam. Meanwhile, tert-butyl 7-(2-chloro-N-(2-cyanoethyl)acetamido)-2,3-dihydro-4H-benzo[b][1,4]oxazine-4-carboxylate (20 mg, 0.052 mmol) was dissolved in DCM (400 μL) and TFA (400 μL) was added. The solution turned light purple and after stirring 5 min at rt the mixture was concentrated under vacuum, and the resulting aniline was redissolved in DCM (0.5 mL). At 0° C., TEA (109 uL, 0.78 mmol) was added to the solution followed by the crude hex-5-ynoyl chloride dissolved in DCM (1 mL). The reaction was stirred for 15 minutes at 0° C., water was added, the mixture partitioned and the aqueous layer extracted with DCM. Combined organic extracts were dried over Na₂SO₄, concentrated, and purified by silica gel chromatography to obtain the title compound (17 mg, 0.047 mmol, 91%). HRMS [M+H]+ calc 374.1193, found 374.1247. ¹H NMR (400 MHz, DMSO) δ 7.94 (s, 1H), 7.03 (d, J=2.5 Hz, 1H), 6.93 (d, J=8.7 Hz, 1H), 4.30 (t, J=4.5 Hz, 2H), 4.07 (s, 2H), 3.94-3.82 (m, 4H), 2.80 (s, 1H), 2.75-2.64 (m, 4H), 2.27-2.19 (m, 2H), 1.76 (p, J=7.2 Hz, 2H). ¹³C NMR (151 MHz, CDCl₃) δ 170.93, 166.76, 147.68, 127.10, 125.66, 119.46, 117.48, 116.71, 83.31, 69.48, 60.39, 46.18, 41.62, 32.78, 23.77, 21.05, 17.76, 16.38, 14.20.

### REFERENCES

1. Almada, A. E., and Wagers, A. J. (2016). Molecular circuitry of stem cell fate in skeletal muscle regeneration, ageing and disease. Nat Rev Mol Cell Biol 17,267-279. 2. Avior, Y., Sagi, I., and Benvenisty, N. (2016). Pluripotent stem cells in disease modelling and drug discovery. Nat Rev Mol Cell Biol 17,170-182. 3. Balchin, D., Hayer-Hartl, M., and Hartl, F. U. (2016). In vivo aspects of protein folding and quality control. Science 353, aac4354. 4. Bellezza, I., Giambanco, I., Minelli, A., and Donato, R. (2018). Nrf2-Keap1 signaling in oxidative and reductive stress. Biochim Biophys Acta Mol Cell Res 1865, 721-733. 5. Buckley, S. M., Aranda-Orgilles, B., Strikoudis, A., Apostolou, E., Loizou, E., Moran-Crusio, K., Farnsworth, C. L., Koller, A. A., Dasgupta, R, Silva, J. C., et al. (2012b). Regulation of pluripotency and cellular reprogramming by the ubiquitin-proteasome system. Cell stem cell 11, 783-798. 6. Cancer Genome Atlas Research, N. (2012). Comprehensive genomic characterization of squamous cell lung cancers. Nature 489, 519-525. 7. Denko, N.C. (2008). Hypoxia, HIF1 and glucose metabolism in the solid tumour. Nat Rev Cancer 8, 705-713. 8. Dialynas, G., Shrestha, O. K., Ponce, J. M., Zwerger, M., Thiemann, D. A., Young, G. H., Moore, S. A., Yu, L., Lammerding, J., and Wallrath, L. L. (2015). Myopathic lamin mutations cause reductive stress and activate the nrf2/keap-1 pathway. PLoS Genet 11, e1005231. 9. Donato, V., Bonora, M., Simoneschi, D., Sartini, D., Kudo, Y., Saraf, A., Florens, L., Washburn, M. P., Stadtfeld, M., Pinton, P., et al. (2017). The TDH-GCN5L1-Fbxo15-KBP axis limits mitochondrial biogenesis in mouse embryonic stem cells. Nat Cell Biol. 10. Ezashi, T., Das, P., and Roberts, R. M. (2005). Low 02 tensions and the prevention of differentiation of hES cells. Proc Natl Acad Sci US A 102, 4783-4788. 11. Furukawa, M., and Xiong, Y. (2005). BTB protein Keap1 targets antioxidant transcription factor Nrf2 for ubiquitination by the Cullin 3-Roc1 ligase. Mol Cell Biol 25, 162-171. 12. Gnarra, J. R., Ward, J. M., Porter, F. D., Wagner, J. R., Devor, D. E., Grinberg, A., Emmert-Buck, M. R., Westphal, H., Klausner, R. D., and Linehan, W. M. (1997). Defective placental vasculogenesis causes embryonic lethality in VHL-deficient mice. Proc Natl Acad Sci USA 94, 9102-9107. 13. Gores, G. J., Flarsheim, C. E., Dawson, T. L., Nieminen, A. L., Herman, B., and Lemasters, J. J. (1989). Swelling, reductive stress, and cell death during chemical hypoxia in hepatocytes. Am J Physiol 257, C347-354. 14. Holmstrom, K. M., and Finkel, T. (2014). Cellular mechanisms and physiological consequences of redox-dependent signalling. Nat Rev Mol Cell Biol 15, 411-421. 15. Kaelin, W. G. (2007). Von Hippel-Lindau disease. Annu Rev Pathol 2, 145-173. 16. Khacho, M., Clark, A., Svoboda, D. S., Azzi, J., MacLaurin, J. G., Meghaizel, C., Sesaki, H., Lagace, D. C., Germain, M., Harper, M. E., et al. (2016). Mitochondrial Dynamics Impacts Stem Cell Identity and Fate Decisions by Regulating a Nuclear Transcriptional Program. Cell stem cell 19,232-247. 17. Nusse, R., and Clevers, H. (2017). Wnt/beta-Catenin Signaling, Disease, and Emerging Therapeutic Modalities. Cell 169,985-999. 18. Ohh, M., Park, C. W., Ivan, M., Hoffman, M. A., Kim, T. Y., Huang, L. E., Pavletich, N., Chau, V., and Kaelin, W. G. (2000). Ubiquitination of hypoxia-inducible factor requires direct binding to the beta-domain of the von Hippel-Lindau protein. Nat Cell Biol 2, 423-427. 19. Rajasekaran, N. S., Connell, P., Christians, E. S., Yan, L. J., Taylor, R. P., Orosz, A., Zhang, X. Q., Stevenson, T. J., Peshock, R. M., Leopold, J A., et al. (2007). Human alpha B-crystallin mutation causes oxido-reductive stress and protein aggregation cardiomyopathy in mice. Cell 130,427-439. 20. Rajasekaran, N. S., Varadharaj, S., Khanderao, G. D., Davidson, C. J., Kannan, S., Firpo, M. A., Zweier, J. L., and Benjamin, I. J. (2011). Sustained activation of nuclear erythroid 2-related factor 2/antioxidant response element signaling promotes reductive stress in the human mutant protein aggregation cardiomyopathy in mice. Antioxid Redox Signal 14, 957-971. 21. Rape, M. (2018). Ubiquitylation at the crossroads of development and disease. Nat Rev Mol Cell Biol 19, 59-70. 22. Rodriguez-Colman, M. J., Schewe, M., Meerlo, M., Stigter, E., Gerrits, J., Pras-Raves, M., Sacchetti, A., Hornsveld, M., Oost, K. C., Snippert, H. J., et al. (2017). Interplay between metabolic identities in the intestinal crypt supports stem cell function. Nature 543, 424-427. 23. Sena, L. A., and Chandel, N. S. (2012). Physiological roles of mitochondrial reactive oxygen species. Mol Cell 48, 158-167. 24. Studer, L., Csete, M., Lee, S. H., Kabbani, N., Walikonis, J., Wold, B., and McKay, R. (2000). Enhanced proliferation, survival, and dopaminergic differentiation of CNS precursors in lowered oxygen. J Neurosci 20, 7377-7383. 25. Suzuki, T., and Yamamoto, M. (2017). Stress-sensing mechanisms and the physiological roles of the Keap1-Nrf2 system during cellular stress. J Biol Chem 292, 16817-16824. 26. Tsai, J. J., Dudakov, J. A., Takahashi, K., Shieh, J. H., Velardi, E., Holland, A. M., Singer, N. V., West, M. L., Smith, O. M., Young, L. F., et al. (2013). Nrt2 regulates haematopoietic stem cell function. Nat Cell Biol 15, 309-316. 27. Vilchez, D., Simic, M. S., and Dillin, A. (2014). Proteostasis and aging of stem cells. Trends Cell Biol 24, 161-170. 28. Wakabayashi, N., Itoh, K., Wakabayashi, J., Motohashi, H., Noda, S., Takahashi, S., Imakado, S., Kotsuji, T., Otsuka, F., Roop, D. R., et a. (2003). Keap1-null mutation leads to postnatal lethality due to constitutive Nrf2 activation. Nat Genet 35, 238-245. 29. Wu, J., Jin, Z., Zheng, H., and Yan, L. J. (2016). Sources and implications ofNADH/NAD(+) redox imbalance in diabetes and its complications. Diabetes Metab Syndr Obes 9, 145-153. 30. Xiao, W., and Loscalzo, J. (2019). Metabolic Responses to Reductive Stress. Antioxid Redox Signal. 31. Yamamoto, M., Kensler, T. W., and Motohashi, H. (2018). The KEAP1-NRF2 System: a Thiol-Based Sensor-Effector Apparatus for Maintaining Redox Homeostasis. Physiol Rev 98, 1169-1203. 32. Yau, R., and Rape, M. (2016). The increasing complexity of the ubiquitin code. Nat Cell Biol 18,579-586. 33. Zhang, D. D., Lo, S. C., Cross, J. V., Templeton, D J., and Hannink, M. (2004). Keap1 is a redox-regulated substrate adaptor protein for a Cul3-dependent ubiquitin ligase complex. Mol Cell Biol 24, 10941-10953.

What is claimed is:

1. A compound having the formula: 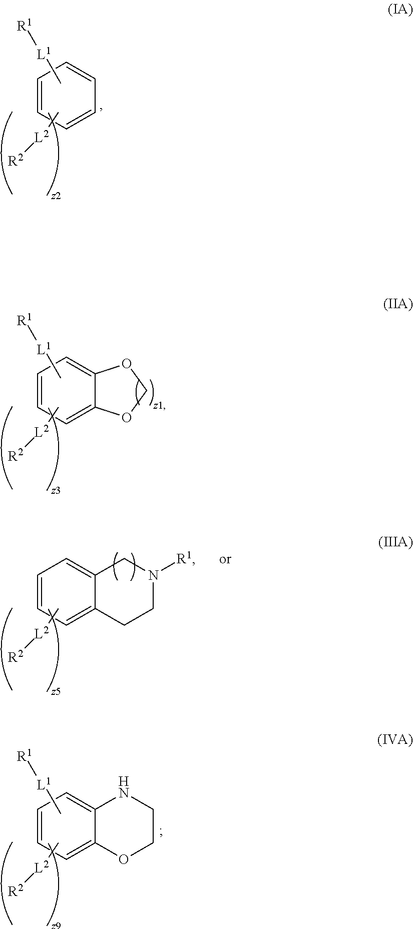
or a pharmaceutically acceptable salt thereof, wherein:
R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰¹ is independently hydrogen, oxo, halogen, —CCl₃, —CBr₃, —CF₃, —CI₃, —CHCl₂, —CHBr₂, —CHF₂, —CHI₂,—CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is an electrophilic moiety;
z1 is 1 or 2;
z2 is 0 to 5;
z3 is 0 to 3;
z4 is 0 or 1; and
z5 and z9 are each independently an integer from 0 to 4.

2. The compound of claim 1, or a pharmaceutically acceptable salt thereof, wherein:
R² is independently halogen, —CCl₃, —CBr₃, —CF₃, —CI₃, —CN, —OH, —NH₂, —COOH, substituted or unsubstituted alkyl, or substituted or unsubstituted heteroalkyl;
L² is independently a bond, —N(R¹⁰²), —C(O)—, substituted or unsubstituted alkylene, or substituted or unsubstituted heteroalkylene;
R¹⁰² is independently hydrogen or unsubstituted alkyl;
L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;
R¹⁰¹ is independently hydrogen, —OH, —NH₂, —COOH, —CONH₂, substituted or unsubstituted alkyl, substituted or unsubstituted aryl, or substituted or unsubstituted heteroaryl;
R¹ is: 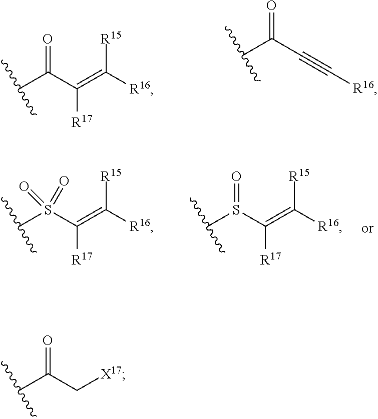
R¹⁵, R¹⁶, and R¹⁷ are independently hydrogen, halogen, —CCl₃, —CBr₃, —CF_{3, —CI}₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X¹⁷ is halogen.

3. The compound of claim 1, or a pharmaceutically acceptable salt thereof, wherein:
R² is independently halogen, —CF₃, unsubstituted C₁-C₃ alkyl, or unsubstituted 2 to 3 membered heteroalkyl;
L² is independently a bond;
L¹ is —N(R¹⁰¹)—; and
R¹⁰¹ is independently hydrogen, substituted or unsubstituted alkyl, substituted or unsubstituted aryl, or substituted or unsubstituted heteroaryl.

4. The compound of claim 1, or a pharmaceutically acceptable salt thereof, wherein:
R² is independently —Cl, —Br, —F, —CF₃, —CH₃, —OCH₃, or —OCH₂CH₃;
L² is independently a bond;
L¹ is —N(R¹⁰¹)—;
R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;
R¹ is: 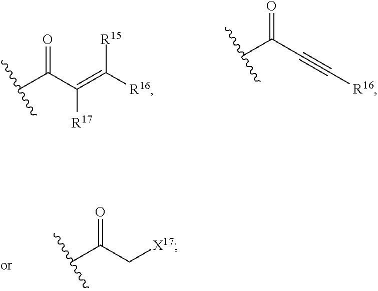
R¹⁵, R¹⁶, and R¹⁷ are independently hydrogen; and
X¹⁷ is halogen.

5. The compound of claim 1, or a pharmaceutically acceptable salt thereof, wherein:
L¹ is —N(R¹⁰¹)—;
R¹⁰¹ is independently hydrogen, —CH₂CH₂CN, 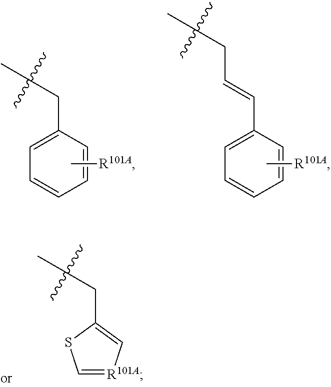
R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is: 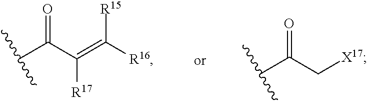
R¹⁵, R¹⁶, and R¹⁷ are independently hydrogen; and
X¹⁷ is halogen.

6. The compound of claim 1, or a pharmaceutically acceptable salt thereof, wherein:
L¹ is —N(R¹⁰¹)—; and
R¹⁰¹ is independently hydrogen, —CH₂CH₂CN,

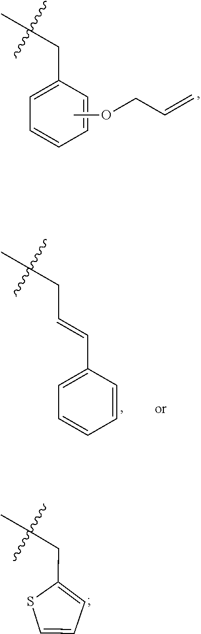

7. A compound having the formula: 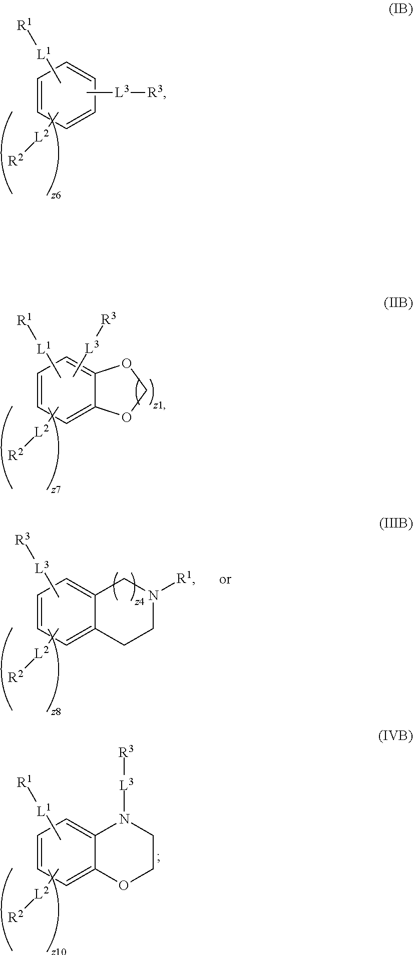
or a pharmaceutically acceptable salt thereof, wherein:
R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is independently a bond, —S(O)₂—, —N(R¹⁰²)—, —O—, —S—, —C(O)—, —C(O)N(R¹⁰²)—, —N(R¹⁰²)C(O)—, —N(R¹⁰²)C(O)NH—, —NHC(O)N(R¹⁰²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰² is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, —S(O)₂—, —N(R¹⁰¹)—, —O—, —S—, —C(O)—, —C(O)N(R¹⁰¹)—, —N(R¹⁰¹)C(O)—, —N(R¹⁰¹)C(O)NH—, —NHC(O)N(R¹⁰¹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁰¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is an electrophilic moiety;
L³ is a bond, —S(O)₂—, —N(R¹⁰³)—, —O—,—S-, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;
R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is a target protein binding moiety;
z1 is 1 or 2;
z4 is 0 or 1;
z6 is 0 to 4;
z7 is 0 to 2; and
z8 and z10 are each independently an integer from 0 to 3.

8. The compound of claim 7, or a pharmaceutically acceptable salt thereof, wherein:
R² is independently halogen, —CCl₃, —CBr₃, —CF₃, —CI₃, —CN, —OH, —NH₂, —COOH, substituted or unsubstituted alkyl, or substituted or unsubstituted heteroalkyl;
L² is independently a bond, —N(R¹⁰²), —C(O)—, substituted or unsubstituted alkylene, or substituted or unsubstituted heteroalkylene;
R¹⁰² is independently hydrogen or unsubstituted alkyl;
L¹ is a bond, —N(R¹⁰¹)—, —O—, —C(O)—, —C(O)N(R¹⁰¹)—, —N(R¹⁰¹)C(O)—, —N(R¹⁰¹)C(O)NH—, or —NHC(O)N(R¹⁰¹)—;
R¹⁰¹ is independently hydrogen, —OH, —NH₂, —COOH, —CONH₂, substituted or unsubstituted alkyl, substituted or unsubstituted aryl, or substituted or unsubstituted heteroaryl;
R¹ is: 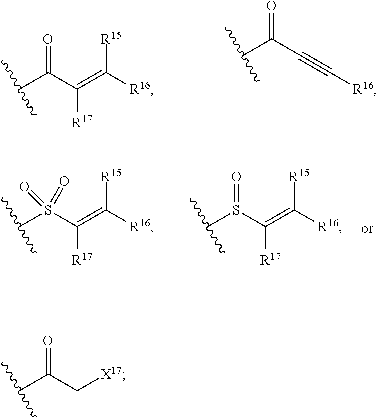
R¹⁵, R¹⁶, and R¹⁷ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹⁷ is halogen;
L³ is a bond, —N(R¹⁰³)—, —O—, —S—, —C(O)—, substituted or unsubstituted alkylene, or substituted or unsubstituted heteroalkylene;
R¹⁰³ is independently hydrogen, —OH, or substituted or unsubstituted alkyl; and
R³ is a target protein binding moiety.

9. The compound of claim 7, or a pharmaceutically acceptable salt thereof, wherein:
R² is independently halogen, —CF₃, unsubstituted C₁-C₃ alkyl or unsubstituted 2 to 3 membered heteroalkyl;
L² is independently a bond;
L¹ is —N(R¹⁰¹)—;
R¹⁰¹ is independently hydrogen, substituted or unsubstituted alkyl, substituted or unsubstituted aryl, or substituted or unsubstituted heteroaryl;
L³ is a bond, substituted or unsubstituted C₁-C₆ alkylene, or substituted or unsubstituted 2 to 6 membered heteroalkylene; and
R³ is a target protein binding moiety.

10. The compound of claim 7, or a pharmaceutically acceptable salt thereof, wherein:
R² is independently —Cl, —Br, —F, —CF₃, —CH₃, —OCH₃, or —OCH₂CH₃;
L² is independently a bond;
L¹ is —N(R¹⁰¹)—;
R¹⁰¹ is independently hydrogen, substituted or unsubstituted C₁-C₆ alkyl, substituted or unsubstituted C₆-C₁₀ aryl, or substituted or unsubstituted 5 to 10 membered heteroaryl;
R¹ is: 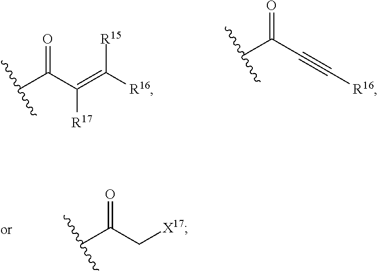
R¹⁵, R¹⁶, and R¹⁷ are independently hydrogen;
X¹⁷ is halogen;
L³ is a bond or substituted or unsubstituted 2 to 6 membered heteroalkylene; and
R³ is a target protein binding moiety.

11. The compound of claim 7, or a pharmaceutically acceptable salt thereof, wherein:
L¹ is —N(R¹⁰¹)—;
R¹⁰¹ is independently hydrogen, —CH₂CH₂CN, 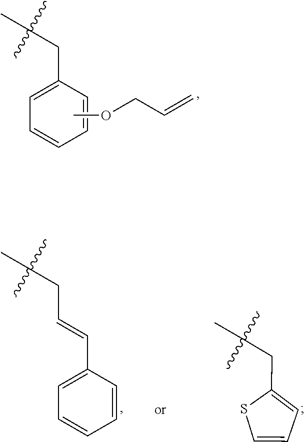
R^{101A} is independently hydrogen, halogen, —OH, —NH₂, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; 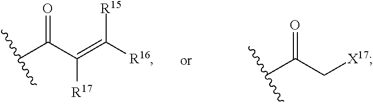
R¹⁵, R¹⁶, and R¹⁷ are independently hydrogen;
X¹⁷ is halogen;
L³ is a bond or substituted or unsubstituted 2 to 6 membered heteroalkylene; and
R³ is a target protein binding moiety.

12. The compound of claim 7, or a pharmaceutically acceptable salt thereof, wherein:
L¹ is N(R¹⁰¹)—;
R¹⁰¹ is independently hydrogen, —CH₂CH₂CN, 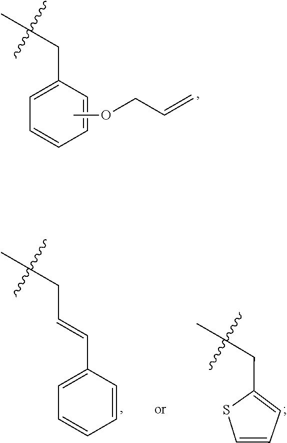
L³ is a bond or substituted or unsubstituted 2 to 6 membered heteroalkylene; and
R³ is a target protein binding moiety.

13. The compound of claim 7, or a pharmaceutically acceptable salt thereof, wherein R³ is a Brd4 binding moiety.

14. The compound of claim 7, or a pharmaceutically acceptable salt thereof, wherein R³ is a K-ras binding moiety.

15. The compound of claim 7, or a pharmaceutically acceptable salt thereof, wherein R³ is a Bruton's tyrosine kinase (BTK) binding moiety.

16. The compound of claim 7, or a pharmaceutically acceptable salt thereof, wherein R³ is an androgen receptor (AR) binding moiety.

17. The compound of claim 7, or a pharmaceutically acceptable salt thereof, wherein R³ is a MYC protein binding moiety.

18. The compound of claim 7, or a pharmaceutically acceptable salt thereof, wherein R³ is an N-MYC protein binding moiety.

19. The compound of claim 7, or a pharmaceutically acceptable salt thereof, wherein R³ is a beta-catenin protein binding moiety.

20. The compound of claim 7, or a pharmaceutically acceptable salt thereof, wherein R³ is a huntingtin (HTT) protein binding moiety.

21. A compound selected from a group consisting of: 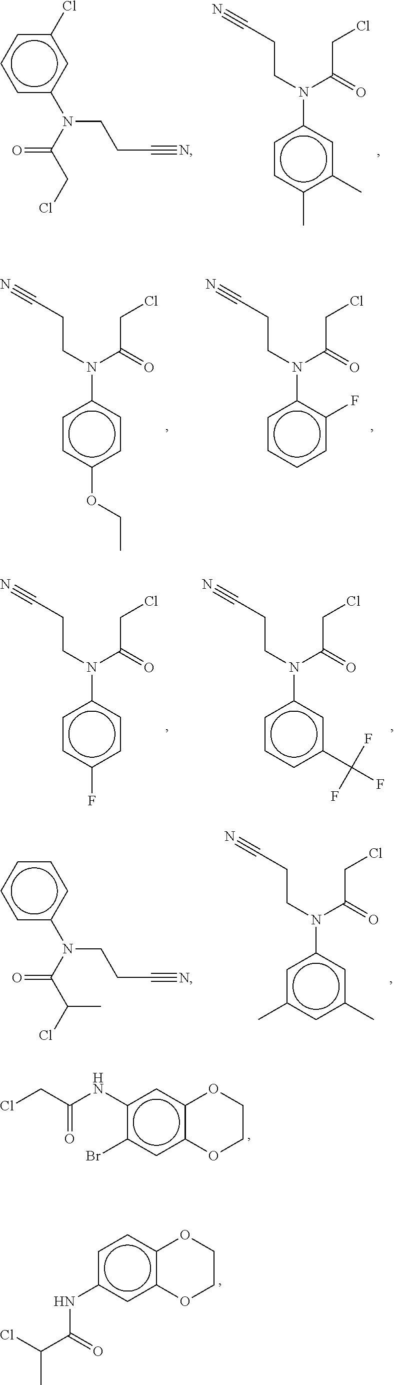 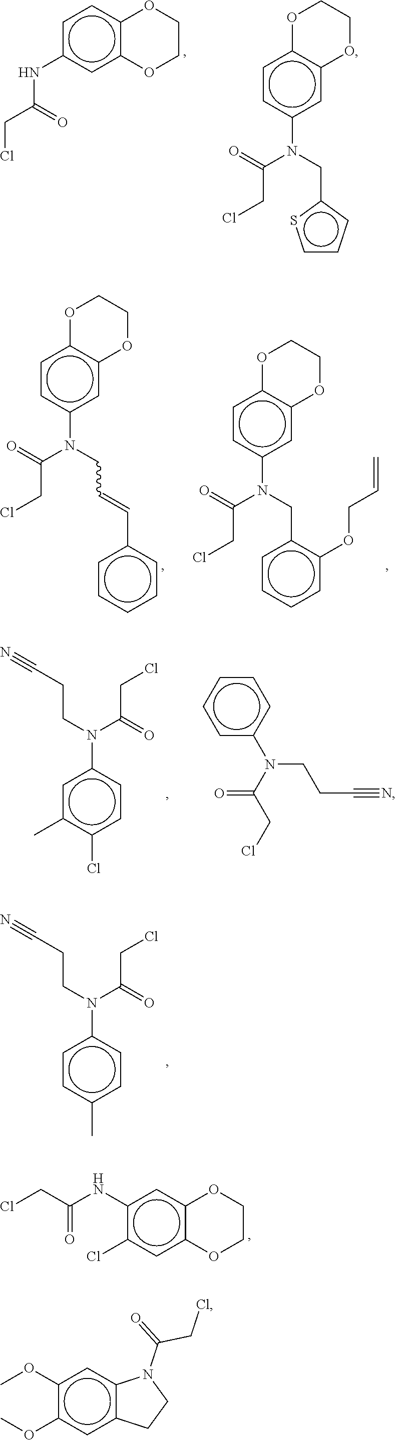 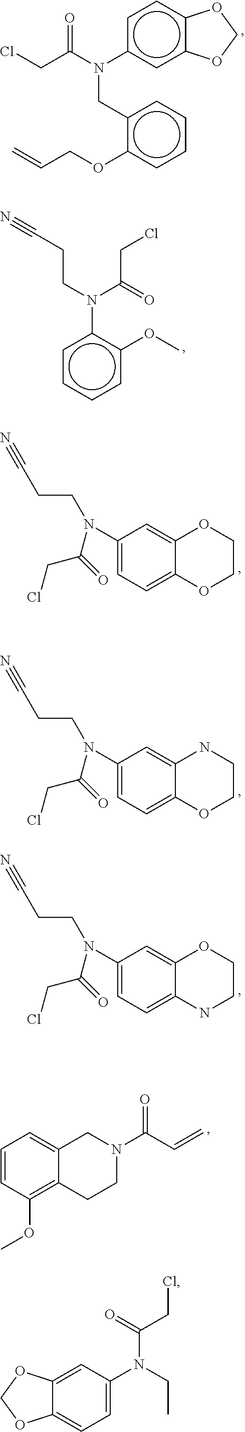 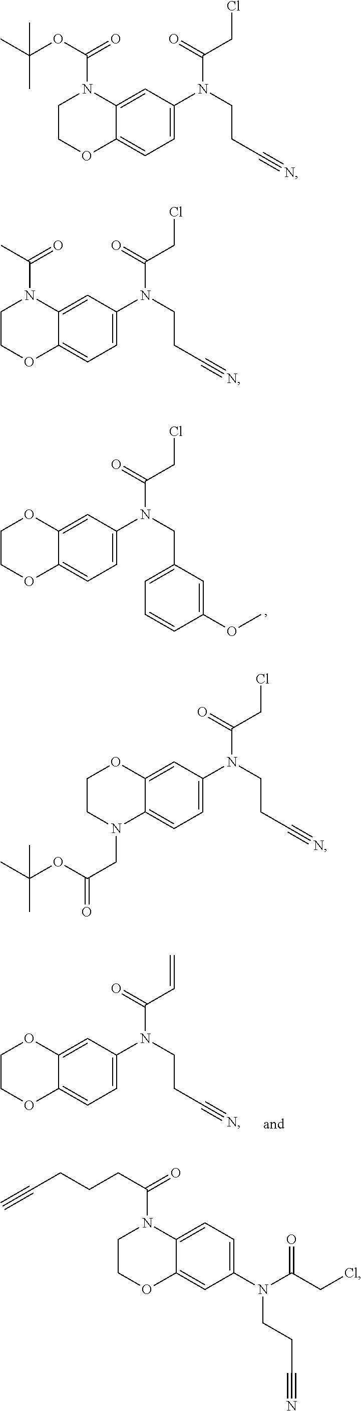
or a pharmaceutically acceptable salt thereof.

22. A compound having the formula: 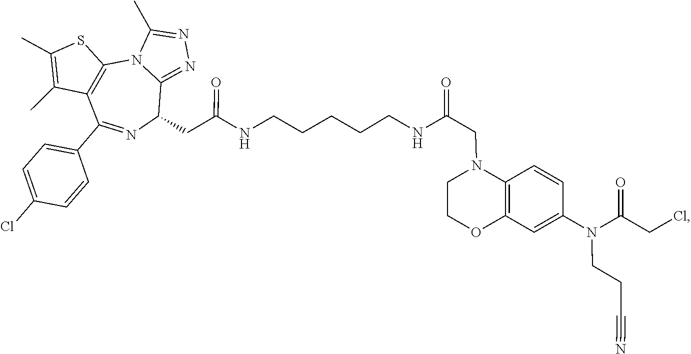 [SMILES: Cc1sc2c(c1C)C(c1ccc(Cl)cc1)=N[C@@H](CC(=O)NCCCCCNC(=O)CN1CCOc3cc(N(CCC#N)C(=O)CCl)ccc31)c1nnc(C)n1-2]
or a pharmaceutically acceptable salt thereof.

23. A pharmaceutical composition comprising the compound of any one of claims 1 to 22 and a pharmaceutically acceptable excipient.

24. A method of treating a disease in a subject in need thereof, the method comprising administering a therapeutically effective amount of FEM1B Cys 186 covalent inhibitor.

25. The method of claim 24, wherein the disease is cancer, obesity, Huntington's disease or diabetes.

26. The method of claim 25, wherein the cancer is a metastatic lung cancer, neuroblastoma, colon cancer, or renal cancer.

27. The method of claim 26, wherein the renal cancer is a KEAP1 mutated renal cancer.

28. The method of claim 25, wherein the diabetes is a type-II diabetes.

29. A method of treating a disease in a subject in need thereof, the method comprising administering a therapeutically effective amount of a compound having the structure: FCIM-L³-R³, wherein
FCIM is a FEM1B Cys 186 covalent inhibitor moiety,
R³ is a target protein binding moiety;
L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;
R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

30. The method of claim 29, wherein the disease is cancer, neurodegenerative disease, mitochondrial disease, and diabetes.

31. The method of claim 30, wherein the cancer is a leukemia or prostate cancer.

32. The method of claim 31, wherein the prostate cancer is a castration-resistant prostate cancer.

33. A FEM1B protein comprising an amino acid corresponding to Cys 186, wherein said amino acid corresponding to Cys 186 is covalently bound to a (i) FEM1B Cys 186 covalent inhibitor; or (ii) a compound having the structure: FCIM-L³-R³, wherein
FCIM is a FEM1B Cys 186 covalent inhibitor moiety,
R³ is a target protein binding moiety;
L³ is a bond, —S(O)₂—, —N(R¹⁰³)—, —O—, —S—, —C(O)—, —C(O)N(R¹⁰³)—, —N(R¹⁰³)C(O)—, —N(R¹⁰³)C(O)NH—, —NHC(O)N(R¹⁰³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or -L^{3A}-L^{3B}-L^{3C}-;
R¹⁰³ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂,—OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{3A} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3B} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{3C} is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
wherein the FCIM is covalently bound to said Cys 186.
